SEQUENCE LISTING (38 sequences)

SEQ ID NO: 1 - DNA, 16947 bp, Artificial sequence
  Synthetic nucleotide
ggtggcaggatatattgtggtgtaaacaaattgacgcttagacaacttaataacacattgcggacgtttttaatgtactggggtggttttggtaccgggccccccctcgaggtcgacggtatcgataagcttgatatcgaattcctgcaggcctcctaggactagttcccaggtcaacatggtggagcacgacactctcgtctactccaagaatatcaaagatacagtctcagaagaccagagggctattgagacttttcaacaaagggtaatatcgggaaacctcctcggattccattgcccagctatctgtcacttcatcgaaaggacagtagaaaaggaagatggcttctacaaatgccatcattgcgataaaggaaaggctatcgttcaagatgcctctaccgacagtggtcccaaagatgagacttttcaacaaagggtaatatcgggaaacctcctcggattccattgcccagctatctgtcacttcatcgaaaggacagtagaaaaggaagatggcttctacaaatgccatcattgcgataaaggaaaggctatcgttcaagatgcctctaccgacagtggtcccaaagatggacccccacccacgaggaacatcgtggaaaaagaagacgttccaaccacgtcttcaaagcaagtggattgatgtgatatctccactgacgtaagggatgacgcacaatcccactatccttcgcaagacccttcctctatataaggaagttcatttcatttggagaggagagacccatatatttcataaagggtcatctggtctgtatcacatagctgattagtcctcatgatgctgggcaagaatgcttgtgggaagatccgcccgcaacctgctcaataacacatgatcaggaagtgacacaatctgcgactattaacaaaaacccaaccacctctaaccttcttcatctcatccttcataatggcaaacatcaacatcccggatgacctggtatccaagtactcggaagatgtaaaggatctcacacaaaaggcaggtagcattccaagttctaaatcattgatcccccaaaccgcattcacccctgatgaattgaggagatctttgaagttctggcaagtgaccaccaaagtggcagcaactgtagcatccgactggactaatttggcgactgcattaaccaacgggacattcagtgcgacgcatctcaggacactgtgtgagctggcattcaacctcaaggccccaactggaacaggcacaatcttcattcatgagattgctgacccgtggaaagggtgcattgatgctaatgctcccgacactgtagcgatacctgcgactgacagtgatgttaatatgtctactgtcagctcaggttctaccgcctctgggacggaagatgcagaggtcaaaatgaaggcgatctcattcctatgctgcacattgctcaggctgtctgtgaaagagccgtcacacatcatgcaagcgatcaacagcatacgacagaggttcggctccctgtacggtgtctcctcccctaccttgaatagtgttacattcaacaggagccaactttctcgcatcaagcaaggcattgaaacatatccctctgctcggggtacagtattctattacaccaggtatgccgatgtcacgcatgggtatactaccaaagagtatgggatatgcaggttcctactgttccaacaccttgaacttgaggggatgcacatctataagatgctcttagctctacttggcgagtggtcaacagttccaattgggctcttgctcacctggatcaggaacccgaggtctaagttggccataaatgagattgtcaagatagtcaaggagcttgataagccggaagtggacaagggctggaaatatgctcggatggtcaataacacattcttccttgatctatcctctagaaggaacacttacctatgtgctgtgttggcctccctgaacaagaagaacgttccgcaaggatcaggggaatatgctgaccccactaacatagctgtgatcaagaatatggaccaatctgtgaaatcacaggttgccacagatgtgaccctgattgagagaatttatgagaaatatctggtgtctgcaggtggagacgaggcaggaacagcatatgctctgtctcgcggagttaagagatcctcgcctcaaagtcaggaaggcggtcaatccggccagtctggaggaacacccatggaagtcgacggagcatctggtagaggagcagcaggtccggccccgaagaagacacgatcaggcttatagatctcagctcggtatcaatagcctacatgagaagatgtaccacgtgccatacgctcctgaacctatgtgtgatcccaatggaccaacaataatagcagagtctgtcataaccctgccgttttaatcaattatcatgtgcttcccatgttgttgtttcaatctattaagaaaaacccaacctcaggagatgaatcgttactcccgtcgttctagacatcccaaccctcctgtccccaatcaggaagaacccgagcgcgacccgaatcacatagatcaagacctcgcagacttggctcaacccttggtcctaaaagaaagacacgcagtaatggcaccaactcagcccagccttagcgatgtgatcaatgaggaaagacaggctcccatcacgtttggtaatcctccagaggtcatggcgaacgctaggctatcagcgctgggttatgacaatctcacggagagggagaagaggatccttgctgtaggaatcagatgcggagaggctgccaaagactatcattcgctcactaccaccaaaaagtggattgaggatgagctgaagtctcaaatggtggctctcgcatcatcaacaaggactctcactgaggctgcatcacttcacacgacctttgcaatgctgcattctccatccattaaaagaaaggcagaagcaatgtctcacatatctcaaggcgaagagagcattgacatttctaagctgaataagactggaatggaggacatctgggtggcaatggagagtgagtcaaaagaggatgcagtagacacgtatctgagaaacattctggaagtggacccgacacaattctatgcaatagacggctggggcctctacctggacttcattcccacttggcattatatagctgcagggaagaattcggcgcaatttaagacgagttatgctgacgagattgtagagcagcgtgctgcattcgagagggtcctctctaagcgccctagggttgagatctaagcctgtatgtcactccactccccaataatgtccacgtcagcctcatgacaccctgctattaagaaaaacccaacaatggagccctctgcagagatccccctcgggaaaggcaaggaaaccgggagaagccctctgatcgcaacaaggaggaagaccgggctggtcaaagccaagataaagaccaattcatccttctctatgttcaggagagcagatactatgcgaacccatgcacagttcaccaacattactgtggggtggaagtcgctttgtcctatcactgcagaaggacaggtcaatgtagccatataccatgagagcacccgagtccctgttctcaatctatggagtcctgtgtcctctagttggaagcacctggcaacaggcagtctaggctttgttagtctgaaccactgtccatatgtggttgaaggaagaatatcgggattcgagggagaagaagcagggttggtgacacttacacttcatcttgacactggattggagagtgatgacatccagaagtgtcgtttattttctgaacatccggagttagcgggtggatcactatatttatacacttcctacacaagtagccccataccggacagcaatctccctgccttaaggactgagatcgatgacactatcgctgcaatttggaggatgagtggagggaagatgtctatcaccacaagccatgctctagggctgattgccaccttcagggttaaaatagagtctttacttggggaatacccggatgatgcattctctgctcaggttgagaatcttgtgaaggattttctccttgtttcatcgacagctgagataaccagatcttacaggacctgccttgcaaaagtgctgtcgtcgatgaccacgtatgacaagaagtactgggacaccatcataacaggtccctcctccatatgacactcgccatcgcagcagctggagctcatattcccatcatcaagcacatctgcaagaataaaaatccgctcccacccttcattcatcctcacaaccaccctccgcccttgttaagaaaaacccaacatggatgctcctatcaatccttcctctcccacccggtcttcctatgagacagccctagagaggcaatggttgatcaaggatgaaccgtcacaccatgttgacttgggatgtcacttcacattagagatcaaagcattctcacccaaaatgattgagttgctggagactggtagcctgactctaggatccgtgtatcaagggctgtacaatttgatcaaggacaaagactgcaaggggctggagctagatcgggacaccacccatgggaacaaattgcatgtcttggcggcagctttcctgtgtgtgctcaggaagtatgagaaccaggtgcggttttacaatgtcaaaaccaaggactctctgacgggcgaaatccagtatatgtctagggtacacatcggagatgataacttggggctcttcgatccagacacctacaggatgataggagggtcatttccggaagggatctaccgactgaacctccgcacacaactcacacccaggggagaagatgacggtctggaaatcataatgggtaccattctaaccgtccccgtgcgcggtcctgctgaccgacagcctctcggagtattgatggacagacaactaagacagttcccggtagggaccaacccttggtcgtccttgggggaggcgaaacctaaagggagatcgttgtcactcctgcgtggaatccttatgtaattctgcatatcacctggttgatatccaataagaatgccactgcccagcattaccccacgattaagaaaaacccaacttacacagacgcaatcatgtctctcattcacctcatctacttccccaccatcctgtctctcatcctaggtacagagggcacaccagcattgacttatggcaacctggctgacaatcagcatggaaatgaggagcaaggggatgttgatatgtatcctctatatgagtgcagtaagaagggtgcaggagtctctagcaataactggtacggagcctgccgaggagcttgctctatgaccaaaaatactactgatgtccacatggagatatattacaggaatgacagtgtcggttggatagatgtattgtctatccagactacagaaatctggaagcactcacatgtaacctggtacggagagtgtgaacacaatacaaaatatgggagctcgaacacagcacctgtctccgtcattctggagcggttggagttccttcacaatgggatgtcatcatggccatacggtcatagcattcatgttcatgacactatctcaagccctgactgtagctattttggtgatgtgtccagatccggatggagattgataatatctaaacgtgctcttgaactgaaatcagatattgcaggagagggctatattgtagaccccgatgtggggtatgtatatcctgttgcggacggaattgggaaaggtaggacctggcatgtatggaaagctagctctgttccgtcttctggctgctacttcaaatcggcaggaattactaattgtaccctactaatggatacattcctctattcatgtccaagcctcaatatagccttctcagctcgggtcgggaaacacataaagagcacgtgcgtcggagatatgaatatatcaacagacggtgtgacttatcagactctcgggagcacggatcaagggtcgatatccacacagttggtatcattatggcatcaatcacaggaagctctcatacagcaacttattttgactatcaacgaggccctaggaaaaattgagacaagctactgtgagtccacttgtgatttgaccgagatgttaatctcccagcataccgagcacccgatggtaattgagacgcctgtcgggccatggctaccatacagcatagacggtaaagtttctgttctcccttgtcaaggtggtcaggatctggtggtgatcaagcctatagagatctgcgcttctccattcatgctcaaagttaaatcactcaagtctctagacacctactggtgggtccctacagatagccacgtggctccgttcacgcaatgcagcaacagggacgatgaagaaatgatcatcaagatacaacggaagaagccattgatatttgaattctggagaggagcctataagcttgattacccgtataactctagtggaatctggctggataaccctggaggtcatattcatagaagctctaaatggttccctacgttggatagtctatcctacagttctcctataaacttgccactgatcagcaaaggtataaggaaacatattcaagccatcgtcagtaccaccgagatcacatctggaggtaacgttacatcatggacccactcgatttcgactattacagaggccatgttcgactcagctgcgatagccgcgggtcgggtcgtgatgtggtggtacacactagaagaaagcgtcaaaaggtatctgactgtagcgttcggtctagtactaactgtcatcaccatttgggctatgtcaaaattattcttccgatcgtcgccaacatatgccagggcgcccacaaatatagaatgggtaaggtcatagacatcgcagtgtccacccacctcatctccatggagcccgatccacaataaaatgccatctatgtatcaaattaatcctatatgtatattaagaaaaacccaattccaatggatccagattatcctgatcttgatcccgagtctcttgatgttctcaacagcctacaagaagggtacgatgaggaggaagaagaggacagcggagtgtcactcaatggaatgggggactaccaccttaagtcagcactcaggtcgtacacagatatgatgaagcacccaatcttcaagaaggagttctccaaggcagtgattaactttggtatatctcacgactcgatgatgtcccagatagaaacaatgttctacatcctgaagagtactccattgtcaatgcaccttgggaccctttatggtgatctcttcactcgtcagagcacgctcggccacagcgaagatgtatttgacattatccaagcagagatacgaactcttcatcctcacctcctcctagagatgacacctgccaaggtcgaggagatgattctgattgctgacaggaagacatatgatgagagggcagcatctgcgttctggtccactctcataacaataaagaattacattccagcttggacagagaagggggcttgtactctggattggcctagtgtgtctctagacaagaagagtgggtacgtgaaagtgatggcaggtaaagatactaccatctacattgggactgatatttgtgtgatagagaagtatcctacagtaaaatgggctccactttcataccttttaaatggagctgacaaggtggcagaaaggctgaatgtgcgttactatagttgtctttgtgatcagcttgatataccagataggataacacttgatatgcttgatgaaatcatcaaagtgggggatgattgtcttcgagagatgggcaatgacggatacaatgtaattggatcctatgaggctttgcttgcaggaataatccaaaggagggacaaccagggactaataccggacagagatttgttgtggagaactactatggaagaatttacatccagccccggcaggaagtatctagaagagtggagcacaatgtttagttcattaagccctgaacagatagcctgtgctcatggactgtataggatctgggggcacccaatagttgatattcttggagggataaagaagatgcaagaggtggcatctgtgaagaagaatcctagacagagtgtactggatgaaataaggaggcaattcaaagaaatgttttttacctcatatcataggatacacaagcactaccctctacatacaatcttgaacagcctggataattcctatatactcaactgtctaaaggataacttgacgatcaacacatcagcaatctcctataacttccaggattgggactgtgtgatggtggagaaaaacttcgaagtgcctttctcttggaacctggttcacaatttgaaagataaagctatttccccgaacaggcaggagatttaccagacgatatcaactcgtggtacaatattcgggtctcagaaccgcagagggatactgaaatcactcacgatggagactgtccaactccgagactttcttcaaggcataaatgacaacgggttggatgacatagacaaaattattggggtctatcctaaagagcgtgagctgaagataaaggctagactgttctccctcatgtcattccggctcagattgtactgtgtgtcaacagaggcattattaggggacaaaatactcaagtatttcccgcaaatcacaatgtctcttgatatgctcacgatgataaagaagatgttcaaagtgtcctcccaaactactagagaggatgattcagtcacagtcatattcaatcttgacttcataaagtggaatctccagatgagacgtaatatatgtgagcctgtcttctcacaattaggtaaactctttggcatggagaatctgttcaataggactcatgagaccttccgagatagcctgatatacctctgttcgggggaaggagtactgtcagccgatccagtctacggagtattcccggacggtacatgggcatgggcgggtgatgagtcggggaaggaagggcttcgtcagaagggttggacaattctgactgtcgttacaatcatgttgattgcaaagcgacacaatgttgatgtatcactaatgggaggaggagacaatcaagtccttggtatcaccatatcaggagtgaccagagatcttcaaggagagctgattacagaatcggcctcactcgcccgaaggacaatcaagcaatttaccgaggacttattaaccactttctccgatctggggctccctttaaaagcatctgaaacatgggtcagtgactctctcttcatgtacaacaaacacatgttttacaagggaattcctctcagatcacctctgaaagccatctcaaggatcttcccgctggcaaacgacacaattatgaccatagacaacatgatcaataatatatcatcaggggtcaaagcagcctgtatgaaggagagacacggaattccacttgtattcatgaaaacactagcatataggaaggttgccgagggagcactgatcctacatccattgacgacttgcttcaggacaccaacattgcctgaaaatggaactgtgacgcgtgacggacggaagcgatcagtgaaggtgactaggaatcagctgagatcattctttacgttatgtatggtgggatcttccactatgggactacctggtactatacatctccctgatatgatcatgaggggattccctgatccattgaccagctacctgtcattcatttcagaattggttcccaagatcatggacccaagactagcgagcatcatcgcaacatcgggtcatatgagcatcaatagaatgacagagtatgcgaaacttgtggaggatcctgtatctataaatcacgacgcccccacacatggactgaatgagatccgacagttatctcgagacttcttgatgaacacgaccctggcagccaacacccatcttaagtcccttttcaccctcctagacagacagagagaaaaagaattctacgacgcactatgttctgctgaggaactggatgtgaaggtgctgcacgaagtagccggagcatcactattcggatatacaaacggcattgcctctcgtattgaccagacaagaacggtgagagcgttaaacgagacagtcaacgttatgaaaaggctagctgatgcagaggcaagatatattgcatatctgttcgctagggacatacataagcatgacctcatcccggatcagtgttccagagtgactgcggacaggtttgtttctgcttctacctttgatatatatataataattatcattaattagtagtaatataatatttcaaatatttttttcaaaataaaagaatgtagtatatagcaattgcttttctgtagtttataagtgtgtatattttaatttataacttttctaatatatgaccaaaacatggtgatgtttaggtatcgatattattcatggaagaagaaggttttaggggtaacagtacctcacccgattgagatgtgtcaagtcacatcggcgacccaaactgcattcgaggatgctgttatatgttggagtgataatctgagcggatcgagaatctacaccacaatgggtctagggaagatatatcaaggttcctacaccaaggaaaggttcaaggccacagacattgctgccgcgtatgggaacgaggacatcctgacaaaagcagtcaggctgcagaagctaattaactggagatatgatgaagggagcagctttgctaggataattcaacttgctctcagagccatcactgatgcaagcactgacggattccatagatctaaggaggagatcaaaggggaattcgatcatagaaggggcatatcaggagatatcagcggaggaattccaaatttccttgtcacacccacctcgcatttctcgtgtaccacatcctcttggatctcacatagcaggggagggaaaaacgagaatatccatttccagagtgtactgatcaacttattatacagagccatggtctatcgaggctcattgcccggtctaccagagatggtctggtattcaaaagagagatgcagccattgcattattgatattgtggaaccgtctccagaacgaacaaccccagctttgtcagagatgccatcagcacctaacaatccttttgcattcatagagtcagtaaatgtgaagttggattatcatcaccaggttgagatagaaaaaggaatggaagagaccaacctactaaattctgagtggggaggctcatctttaacaagtgtggatgaggctggactggtcttgtttctgatgctgattggatcccgtcaagtgtcagaatcgtttttgttattgatgagggagaagatagaacctcgaccgctactgcaagcagcattgaacagagtcatccttgtcaggaaactcggattggataggttgttccctataagaaacaccaaatgcatcaatcttatactggggtctgacagaggaatcactgcatgccatgacggctttggcctggaactgcacgggggatcctgggagaaagggatagagtgtgacacctcctttctttacaatgatactcacctcaagccctgtaaattgcatgttaaccttcatcttcaaaatgtgcctctccagctggccttggcgcatgcttcctataatcctgatgtttgtgagtgtcttgagtgcagagccatcattgaagatagagtctctaagaggatggtagggcaatataagagctggagatgccagtatcatgcggtcctgtccttctctccaatcatcatgagggtccacagtgagaggataatcaaaaatgagggagctatatatgaaggggatctgcatgtcccttatgtcgaggagatcattgctctggagaactcacaaaaattggcaatagatgtgacgtcatggagactgccgctgctgatgcacgcaacatgggagtctatcctgcctcagctgtatattactatgaaggcctgcctggtttgtcttcccttggagggtgtgattgtggatgatgacctggcactgatcaatctggtgtccaaggtgttggtcgatctgaggaggaccatccaggtctatatcagagcagggacgttctccgggtcggagatcaacaataagtatgacaacatcaggctgcttccagaggatcttagggacagcattcacatcctaacaaccacagacaaaccagatcctgctgcaactgtccaactggcagcggatgacacatcgcctcttgactgtgctgtgtaccatctcatctggggcgatacctggggcatgacgaggaaggtgcacggaagaattctcattccatcctctcgtgtgacaactggattcgcaggggtcatggtgacagatcactacgcaactgtggcagtattggagcttgctggtgctgttaacctgtgggagaaaaagctcattgatcttgaacatagaaacaacctcgatgttccgttgagcatgaatcgtgtgttggcaggttctagactcgggacccgggggataagatgggctcaatggacgtcgcctgcaggcctagagatcgttgtcaggaaagtgagggcaaagctgtcatcaatctcgggaaaccctggaaatgccgccaaatggaggaggaactgccagaaggtcctcaaggccttgatgatctccctgtatgctcattcgggcgaggacatgatgaggaatgcaagcggccttgtcaaggtgaacatacatgggaccctttcctctgtcaccgctgtgtgcgacccgtcggcggagagcagagtgcagagggcacagttcctgttcctaaaagaaaaggctcccaatgggcctttcattctaaggagcaggttggagaggcgcataaatctcctctcagttgtgtccgacctgctcgggtgatcaagaagtattaagaaaaaccctcatgttcacgtggttagaggggttacacccatactacatatcaaggtctttgacaatagtgtatttcgtgagaatgccatgtttttctgggtctctgggtcggcatggcatctccacctcctcgcggtccgacctgggcatccgaaggaggacgtccactcggatggctaagggagagctcgaattcggtacgctgaaatcaccagtctctctctacaaatctatctctctctattttctccataaataatgtgtgagtagtttcccgataagggaaattagggttcttatagggtttcgctcatgtgttgagcatataagaaacccttagtatgtatttgtatttgtaaaatacttctatcaataaaatttctaattcctaaaaccaaaatccagtactaaaatccagatccccaattcactggccgtcgttttacaacgtcgtgactgggaaaaccctggcgttacccaacttaatcgcctgtttaaactatcagtgtttgacagggacctgcagcccggccgggggatccactagcagattgtcgtttcccgccttcagtttaaactatcagtgtttgacaggatatattggcgggtaaacctaagagaaaagagcgtttattagaataatcggatatttaaaagggcgtgaaaaggtttatccgttcgtccatttgtatgtgcatgccaaccacaggagatctcagtaaagcgctggctgaacccccagccggaactgaccccacaaggccctagcgtttgcaatgcaccaggtcatcattgacccaggcgtgttccaccaggccgctgcctcgcaactcttcgcaggcttcgccgacctgctcgcgccacttcttcacgcgggtggaatccgatccgcacatgaggcggaaggtttccagcttgagcgggtacggctcccggtgcgagctgaaatagtcgaacatccgtcgggccgtcggcgacagcttgcggtacttctcccatatgaatttcgtgtagtggtcgccagcaaacagcacgacgatttcctcgtcgatcaggacctggcaacgggacgttttcttgccacggtccaggacgcggaagcggtgcagcagcgacaccgattccaggtgcccaacgcggtcggacgtgaagcccatcgccgtcgcctgtaggcgcgacaggcattcctcggccttcgtgtaataccggccattgatcgaccagcccaggtcctggcaaagctcgtagaacgtgaaggtgatcggctcgccgataggggtgcgcttcgcgtactccaacacctgctgccacaccagttcgtcatcgtcggcccgcagctcgacgccggtgtaggtgatcttcacgtccttgttgacgtggaaaatgaccttgttttgcagcgcctcgcgcgggattttcttgttgcgcgtggtgaacagggcagagcgggccgtgtcgtttggcatcgctcgcatcgtgtccggccacggcgcaatatcgaacaaggaaagctgcatttccttgatctgctgcttcgtgtgtttcagcaacgcggcctgcttggcctcgctgacctgttttgccaggtcctcgccggcggtttttcgcttcttggtcgtcatagttcctcgcgtgtcgatggtcatcgacttcgccaaacctgccgcctcctgttcgagacgacgcgaacgctccacggcggccgatggcgcgggcagggcagggggagccagttgcacgctgtcgcgctcgatcttggccgtagcttgctggaccatcgagccgacggactggaaggtttcgcggggcgcacgcatgacggtgcggcttgcgatggtttcggcatcctcggcggaaaaccccgcgtcgatcagttcttgcctgtatgccttccggtcaaacgtccgattcattcaccctccttgcgggattgccccgactcacgccggggcaatgtgcccttattcctgatttgacccgcctggtgccttggtgtccagataatccaccttatcggcaatgaagtcggtcccgtagaccgtctggccgtccttctcgtacttggtattccgaatcttgccctgcacgaataccagcgaccccttgcccaaatacttgccgtgggcctcggcctgagagccaaaacacttgatgcggaagaagtcggtgcgctcctgcttgtcgccggcatcgttgcgccacatctaggtactaaaacaattcatccagtaaaatataatattttattttctcccaatcaggcttgatccccagtaagtcaaaaaatagctcgacatactgttcttccccgatatcctccctgatcgaccggacgcagaaggcaatgtcataccacttgtccgccctgccgcttctcccaagatcaataaagccacttactttgccatctttcacaaagatgttgctgtctcccaggtcgccgtgggaaaagacaagttcctcttcgggcttttccgtctttaaaaaatcatacagctcgcgcggatctttaaatggagtgtcttcttcccagttttcgcaatccacatcggccagatcgttattcagtaagtaatccaattcggctaagcggctgtctaagctattcgtatagggacaatccgatatgtcgatggagtgaaagagcctgatgcactccgcatacagctcgataatcttttcagggctttgttcatcttcatactcttccgagcaaaggacgccatcggcctcactcatgagcagattgctccagccatcatgccgttcaaagtgcaggacctttggaacaggcagctttccttccagccatagcatcatgtccttttcccgttccacatcataggtggtccctttataccggctgtccgtcatttttaaatataggttttcattttctcccaccagcttatataccttagcaggagacattccttccgtatcttttacgcagcggtatttttcgatcagttttttcaattccggtgatattctcattttagccatttattatttccttcctcttttctacagtatttaaagataccccaagaagctaattataacaagacgaactccaattcactgttccttgcattctaaaaccttaaataccagaaaacagctttttcaaagttgttttcaaagttggcgtataacatagtatcgacggagccgattttgaaaccacaattatgggtgatgctgccaactcgagagcgggccgggagggttcgagaagggggggcaccccccttcggcgtgcgcggtcacgcgcacagggcgcagccctggttaaaaacaaggtttataaatattggtttaaaagcaggttaaaagacaggttagcggtggccgaaaaacgggcggaaacccttgcaaatgctggattttctgcctgtggacagcccctcaaatgtcaataggtgcgcccctcatctgtcagcactctgcccctcaagtgtcaaggatcgcgcccctcatctgtcagtagtcgcgcccctcaagtgtcaataccgcagggcacttatccccaggcttgtccacatcatctgtgggaaactcgcgtaaaatcaggcgttttcgccgatttgcgaggctggccagctccacgtcgccggccgaaatcgagcctgcccctcatctgtcaacgccgcgccgggtgagtcggcccctcaagtgtcaacgtccgcccctcatctgtcagtgagggccaagttttccgcgaggtatccacaacgccggcggccggccgcggtgtctcgcacacggcttcgacggcgtttctggcgcgtttgcagggccatagacggccgccagcccagcggcgagggcaaccagcccggtgagctctagtggactgatgggctgcctgtatcgagtggtgattttgtgccgagctgccggtcggggagctgttggctggct

SEQ ID NO: 2 - DNA, 12338 bp, Artificial sequence
  Synthetic nucleotide
ttctactttatgcgactaaaacacgcgacaagaaaacgccaggaaaagggcagggcggcagcctgtcgcgtaacttaggacttgtgcgacatgtcgttttcagaagacggctgcactgaacgtcagaagccgactgcactatagcagcggaggggttggatcaaagtactttaaagtactttaaagtactttaaagtactttgatcccgaggggaaccctgtggttggcatgcacatacaaatggacgaacggataaaccttttcacgcccttttaaatatccgattattctaataaacgctcttttctcttaggtttacccgccaatatatcctgtcaaacactgatagtttaaactgaaggcgggaaacgacaatctgatccaagctcaagctccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgacatgattacgaattgccactttgtacaagaaagctggtgcggccgcgtcactggattttggttttaggaattagaaattttattgatagaagtattttacaaatacaaatacatactaagggtttcttatatgctcaacacatgagcgaaaccctataagaaccctaattcccttatctgggaactactcacacattattctggagaaaaatagagagagatagatttgtagagagagactggtgatttttgcggactctagactaggtggatcccgggcccgcggtaccgtcgactgcagaattcgaagcttgagctcactagtttagatctcaaccctagggcgcttagagaggaccctctcgaatgcagcacgctgctctacaatctcgtcagcataactcgtcttaaattgcgccgaattcttccctgcagctatataatgccaagtgggaatgaagtccaggtagaggccccagccgtctattgcatagaattgtgtcgggtccacttccagaatgtttctcagatacgtgtctactgcatcctcttttgactcactctccattgccacccagatgtcctccattccagtcttattcagcttagaaatgtcaatgctctcttcgccttgagatatgtgagacattgcttctgcctttcttttaatggatggagaatgcagcattgcaaaggtcgtgtgaagtgatgcagcctcagtgagagtccttgttgatgatgcgagagccaccatttgagacttcagctcatcctcaatccactttttggtggtagtgagcgaatgatagtctttggcagcctctccgcatctgattcctacagcaaggatcctcttctccctctccgtgagattgtcataacccagcgctgatagcctagcgttcgccatgacctctggaggattaccaaacgtgatgggagcctgtctttcctcattgatcacatcgctaaggctgggctgagttggtgccattactgcgtgtctttcttttaggaccaagggttgagccaagtctgcgaggtcttgatctatgtgatttgggtcgcgctcgggttcttcctgattggggacaggagggttgggatgtctagaacgacgggagtaacgattcatcctaggggccatggctatcgttcgtaaatggtgaaaattttcagaaaattgcttttgctttaaaagaaatgatttaaattgctgcaatagaagtagaatgcttgattgcttgagattcgtttgttttgtatatgttgtgttgagaactctcgacgtcctctccaaatgaaatgaacttccttatatagaggaagggtcttgcgaaggatagtgggattgtgcgtcatcccttacgtcagtggagatatcacatcaatccacttgctttgaagacgtggttggaacgtcttctttttccacgatgctcctcgtgggtgggggtccatctttgggaccactgtcggcagaggcatcttcaacgatggcctttcctttatcgcaatgatggcatttgtaggagccaccttccttttccactatcttcacaataaagtgacagatagctgggcaatggaatccgaggaggtttccggatattaccctttgttgaaaagtctcaattgccctttggtcttctgagactgtatctttgatatttttggagtagacaagtgtgtcgtgctccaccatgttatcacatcaatccacttgctttgaagacgtggttggaacgtcttctttttccacgatgctcctcgtgggtgggggtccatctttgggaccactgtcggcagaggcatcttcaacgatggcctttcctttatcgcaatgatggcatttgtaggagccaccttccttttccactatcttcacaataaagtgacagatagctgggcaatggaatccgaggaggtttccggatattaccctttgttgaaaagtctcaattgccctttggtcttctgagactgtatctttgatatttttggagtagacaagtgtgtcgtgctccacatgttgaccggttggccagcttttttgtacaaacttggaattcgtagcttgcatgcctgcaggtcaacatggtggagcacgacacacttgtctactccaaaaatatcaaagatacagtctcagaagaccaaagggcaattgagacttttcaacaaagggtaatatccggaaacctcctcggattccattgcccagctatctgtcactttattgtgaagatagtggaaaaggaaggtggctcctacaaatgccatcattgcgataaaggaaaggccatcgttgaagatgcctctgccgacagtggtcccaaagatggacccccacccacgaggagcatcgtggaaaaagaagacgttccaaccacgtcttcaaagcaagtggattgatgtgataacatggtggagcacgacacacttgtctactccaaaaatatcaaagatacagtctcagaagaccaaagggcaattgagacttttcaacaaagggtaatatccggaaacctcctcggattccattgcccagctatctgtcactttattgtgaagatagtggaaaaggaaggtggctcctacaaatgccatcattgcgataaaggaaaggccatcgttgaagatgcctctgccgacagtggtcccaaagatggacccccacccacgaggagcatcgtggaaaaagaagacgttccaaccacgtcttcaaagcaagtggattgatgtgataacatggtggagcacgacacacttgtctactccaaaaatatcaaagatacagtctcagaagaccaaagggcaattgagacttttcaacaaagggtaatatccggaaacctcctcggattccattgcccagctatctgtcactttattgtgaagatagtggaaaaggaaggtggctcctacaaatgccatcattgcgataaaggaaaggctatcgttcaagatgcctctaccgacagtggtcccaaagatggacccccacccacgaggagcatcgtggaaaaagaagacgttccaaccacgtcttcaaagcaagtggattgatgtgatatctccactgacgtaagggatgacgcacaatcccactatccttcgcaagacccttcctctatataaggaagttcatttcatttggagaggacctcgagaattaattctcaacacaacatatacaaaacaaacgaatctcaagcaatcaagcattctacttctattgcagcaatttaaatcatttcttttaaagcaaaagcaattttctgaaaattttcaccatttacgaacgggggaaggcctatggcaaacatcaacatcccggatgacctggtatccaagtactcggaagatgtaaaggatctcacacaaaaggcaggtagcattccaagttctaaatcattgatcccccaaaccgcattcacccctgatgaattgaggagatctttgaagttctggcaagtgaccaccaaagtggcagcaactgtagcatccgactggactaatttggcgactgcattaaccaacgggacattcagtgcgacgcatctcaggacactgtgtgagctggcattcaacctcaaggccccaactggaacaggcacaatcttcattcatgagattgctgacccgtggaaagggtgcattgatgctaatgctcccgacactgtagcgatacctgcgactgacagtgatgttaatatgtctactgtcagctcaggttctaccgcctctgggacggaagatgcagaggtcaaaatgaaggcgatctcattcctatgctgcacattgctcaggctgtctgtgaaagagccgtcacacatcatgcaagcgatcaacagcatacgacagaggttcggctccctgtacggtgtctcctcccctaccttgaatagtgttacattcaacaggagccaactttctcgcatcaagcaaggcattgaaacatatccctctgctcggggtacagtattctattacaccaggtatgccgatgtcacgcatgggtatactaccaaagagtatgggatatgcaggttcctactgttccaacaccttgaacttgaggggatgcacatctataagatgctcttagctctacttggcgagtggtcaacagttccaattgggctcttgctcacctggatcaggaacccgaggtctaagttggccataaatgagattgtcaagatagtcaaggagcttgataagccggaagtggacaagggctggaaatatgctcggatggtcaataacacattcttccttgatctatcctctagaaggaacacttacctatgtgctgtgttggcctccctgaacaagaagaacgttccgcaaggatcaggggaatatgctgaccccactaacatagctgtgatcaagaatatggaccaatctgtgaaatcacaggttgccacagatgtgaccctgattgagagaatttatgagaaatatctggtgtctgcaggtggagacgaggcaggaacagcatatgctctgtctcgcggagttaagagatcctcgcctcaaagtcaggaaggcggtcaatccggccagtctggaggaacacccatggaagtcgacggagcatctggtagaggagcagcaggtccggccccgaagaagacacgatcaggcttatagggcgcgcccagatctgctcaacaatctagctagagtttgctcctatctatatgtaataaggtatgctgatatgcactattcaaataggagcattagctatgtttgttaatgtcactttatgttatgtgggtaagtcacctaagacactccacgtacctacttgttgtctcttaccgcggctttaataaatcttctgcccttgttccatatttactaattatccctttcttcactaaaagaaaattgttatcattaagtattagtctttagaacatatgaggtctttaattgggtaggttttacaaattaactaatataaaatgtcataaaatccacgtggttaaacaaatgcagaaaatcgacgtcgtctattggaccgacagttgctattaatataatgggccaccatagtagactgacaaataaattacctgacaacatcgtttcacaaaaaaacaaacacaaaaagggagtgcattttccagggcatttttgtaataaaaaacagttaaaagggagtgcaatagaaatataggggtgtggaaatagtgatttgagcacgtcttgaagcgaattagcttggcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgctagagcaattcggcgttaattcagtacattaaaaacgtccgcaatgtgttattaagttgtctaagcgtcaatttgtttacaccacaatatatcctgccaccagccagccaacagctccccgaccggcagctcggcacaaaatcaccactcgatacaggcagcccatcagtccgggacggcgtcagcgggagagccgttgtaaggcggcagactttgctcatgttaccgatgctattcggaagaacggcaactaagctgccgggtttgaaacacggatgatctcgcggagggtagcatgttgattgtaacgatgacagagcgttgctgcctgtgatcaattcgggcacgaacccagtggacataagcctcgttcggttcgtaagctgtaatgcaagtagcgtaactgccgtcacgcaactggtccagaaccttgaccgaacgcagcggtggtaacggcgcagtggcggttttcatggcttgttatgactgtttttttggggtacagtctatgcctcgggcatccaagcagcaagcgcgttacgccgtgggtcgatgtttgatgttatggagcagcaacgatgttacgcagcagggcagtcgccctaaaacaaagttaaacatcatgggggaagcggtgatcgccgaagtatcgactcaactatcagaggtagttggcgtcatcgagcgccatctcgaaccgacgttgctggccgtacatttgtacggctccgcagtggatggcggcctgaagccacacagtgatattgatttgctggttacggtgaccgtaaggcttgatgaaacaacgcggcgagctttgatcaacgaccttttggaaacttcggcttcccctggagagagcgagattctccgcgctgtagaagtcaccattgttgtgcacgacgacatcattccgtggcgttatccagctaagcgcgaactgcaatttggagaatggcagcgcaatgacattcttgcaggtatcttcgagccagccacgatcgacattgatctggctatcttgctgacaaaagcaagagaacatagcgttgccttggtaggtccagcggcggaggaactctttgatccggttcctgaacaggatctatttgaggcgctaaatgaaaccttaacgctatggaactcgccgcccgactgggctggcgatgagcgaaatgtagtgcttacgttgtcccgcatttggtacagcgcagtaaccggcaaaatcgcgccgaaggatgtcgctgccgactgggcaatggagcgcctgccggcccagtatcagcccgtcatacttgaagctagacaggcttatcttggacaagaagaagatcgcttggcctcgcgcgcagatcagttggaagaatttgtccactacgtgaaaggcgagatcaccaaggtagtcggcaaataatgtctagctagaaattcgttcaagccgacgccgcttcgcggcgcggcttaactcaagcgttagatgcactaagcacataattgctcacagccaaactatcaggtcaagtctgcttttattatttttaagcgtgcataataagccctacacaaattgggagatatatcatgcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagggtgccttgatgtgggcgccggcggtcgagtggcgacggcgcggcttgtccgcgccctggtagattgcctggccgtaggccagccatttttgagcggccagcggccgcgataggccgacgcgaagcggcggggcgtagggagcgcagcgaccgaagggtaggcgctttttgcagctcttcggctgtgcgctggccagacagttatgcacaggccaggcgggttttaagagttttaataagttttaaagagttttaggcggaaaaatcgccttttttctcttttatatcagtcacttacatgtgtgaccggttcccaatgtacggctttgggttcccaatgtacgggttccggttcccaatgtacggctttgggttcccaatgtacgtgctatccacaggaaagagaccttttcgacctttttcccctgctagggcaatttgccctagcatctgctccgtacattaggaaccggcggatgcttcgccctcgatcaggttgcggtagcgcatgactaggatcgggccagcctgccccgcctcctccttcaaatcgtactccggcaggtcatttgacccgatcagcttgcgcacggtgaaacagaacttcttgaactctccggcgctgccactgcgttcgtagatcgtcttgaacaaccatctggcttctgccttgcctgcggcgcggcgtgccaggcggtagagaaaacggccgatgccgggatcgatcaaaaagtaatcggggtgaaccgtcagcacgtccgggttcttgccttctgtgatctcgcggtacatccaatcagctagctcgatctcgatgtactccggccgcccggtttcgctctttacgatcttgtagcggctaatcaaggcttcaccctcggataccgtcaccaggcggccgttcttggccttcttcgtacgctgcatggcaacgtgcgtggtgtttaaccgaatgcaggtttctaccaggtcgtctttctgctttccgccatcggctcgccggcagaacttgagtacgtccgcaacgtgtggacggaacacgcggccgggcttgtctcccttcccttcccggtatcggttcatggattcggttagatgggaaaccgccatcagtaccaggtcgtaatcccacacactggccatgccggccggccctgcggaaacctctacgtgcccgtctggaagctcgtagcggatcacctcgccagctcgtcggtcacgcttcgacagacggaaaacggccacgtccatgatgctgcgactatcgcgggtgcccacgtcatagagcatcggaacgaaaaaatctggttgctcgtcgcccttgggcggcttcctaatcgacggcgcaccggctgccggcggttgccgggattctttgcggattcgatcagcggccgcttgccacgattcaccggggcgtgcttctgcctcgatgcgttgccgctgggcggcctgcgcggccttcaacttctccaccaggtcatcacccagcgccgcgccgatttgtaccgggccggatggtttgcgaccgctcacgccgattcctcgggcttgggggttccagtgccattgcagggccggcagacaacccagccgcttacgcctggccaaccgcccgttcctccacacatggggcattccacggcgtcggtgcctggttgttcttgattttccatgccgcctcctttagccgctaaaattcatctactcatttattcatttgctcatttactctggtagctgcgcgatgtattcagatagcagctcggtaatggtcttgccttggcgtaccgcgtacatcttcagcttggtgtgatcctccgccggcaactgaaagttgacccgcttcatggctggcgtgtctgccaggctggccaacgttgcagccttgctgctgcgtgcgctcggacggccggcacttagcgtgtttgtgcttttgctcattttctctttacctcattaactcaaatgagttttgatttaatttcagcggccagcgcctggacctcgcgggcagcgtcgccctcgggttctgattcaagaacggttgtgccggcggcggcagtgcctgggtagctcacgcgctgcgtgatacgggactcaagaatgggcagctcgtacccggccagcgcctcggcaacctcaccgccgatgcgcgtgcctttgatcgcccgcgacacgacaaaggccgcttgtagccttccatccgtgacctcaatgcgctgcttaaccagctccaccaggtcggcggtggcccatatgtcgtaagggcttggctgcaccggaatcagcacgaagtcggctgccttgatcgcggacacagccaagtccgccgcctggggcgctccgtcgatcactacgaagtcgcgccggccgatggccttcacgtcgcggtcaatcgtcgggcggtcgatgccgacaacggttagcggttgatcttcccgcacggccgcccaatcgcgggcactgccctggggatcggaatcgactaacagaacatcggccccggcgagttgcagggcgcgggctagatgggttgcgatggtcgtcttgcctgacccgcctttctggttaagtacagcgataaccttcatgcgttccccttgcgtatttgtttatttactcatcgcatcatatacgcagcgaccgcatgacgcaagctgttttactcaaatacacatcacctttttagacggcggcgctcggtttcttcagcggccaagctggccggccaggccgccagcttggcatcagacaaaccggccaggatttcatgcagccgcacggttgagacgtgcgcgggcggctcgaacacgtacccggccgcgatcatctccgcctcgatctcttcggtaatgaaaaacggttcgtcctggccgtcctggtgcggtttcatgcttgttcctcttggcgttcattctcggcggccgccagggcgtcggcctcggtcaatgcgtcctcacggaaggcaccgcgccgcctggcctcggtgggcgtcacttcctcgctgcgctcaagtgcgcggtacagggtcgagcgatgcacgccaagcagtgcagccgcctctttcacggtgcggccttcctggtcgatcagctcgcgggcgtgcgcgatctgtgccggggtgagggtagggcgggggccaaacttcacgcctcgggccttggcggcctcgcgcccgctccgggtgcggtcgatgattagggaacgctcgaactcggcaatgccggcgaacacggtcaacaccatgcggccggccggcgtggtggtgtcggcccacggctctgccaggctacgcaggcccgcgccggcctcctggatgcgctcggcaatgtccagtaggtcgcgggtgctgcgggccaggcggtctagcctggtcactgtcacaacgtcgccagggcgtaggtggtcaagcatcctggccagctccgggcggtcgcgcctggtgccggtgatcttctcggaaaacagcttggtgcagccggccgcgtgcagttcggcccgttggttggtcaagtcctggtcgtcggtgctgacgcgggcatagcccagcaggccagcggcggcgctcttgttcatggcgtaatgtctccggttctagtcgcaagta

SEQ ID NO: 3 - DNA, 10839 bp, Artificial sequence
  Synthetic nucleotide
ggtggcaggatatattgtggtgtaaacaaattgacgcttagacaacttaataacacattgcggacgtttttaatgtactggggtggttttggtaccgggccccccctcgaggtcgacggtatcgataagcttgatatcgaattcctgcaggcctcctaggactagttccaggtcaacatggtggagcacgacactctcgtctactccaagaatatcaaagatacagtctcagaagaccagagggctattgagacttttcaacaaagggtaatatcgggaaacctcctcggattccattgcccagctatctgtcacttcatcgaaaggacagtagaaaaggaagatggcttctacaaatgccatcattgcgataaaggaaaggctatcgttcaagatgcctctaccgacagtggtcccaaagatgagacttttcaacaaagggtaatatcgggaaacctcctcggattccattgcccagctatctgtcacttcatcgaaaggacagtagaaaaggaagatggcttctacaaatgccatcattgcgataaaggaaaggctatcgttcaagatgcctctaccgacagtggtcccaaagatgagacttttcaacaaagggtaatatcgggaaacctcctcggattccattgcccagctatctgtcacttcatcgaaaggacagtagaaaaggaagatggcttctacaaatgccatcattgcgataaaggaaaggctatcgttcaagatgcctctaccgacagtggtcccaaagatggacccccacccacgaggaacatcgtggaaaaagaagacgttccaaccacgtcttcaaagcaagtggattgatgtgatatctccactgacgtaagggatgacgcacaatcccactatccttcgcaagacccttcctctatataaggaagttcatttcatttggagaggatggatccagattatcctgatcttgatcccgagtctcttgatgttctcaacagcctacaagaagggtacgatgaggaggaagaagaggacagcggagtgtcactcaatggaatgggggactaccaccttaagtcagcactcaggtcgtacacagatatgatgaagcacccaatcttcaagaaggagttctccaaggcagtgattaactttggtatatctcacgactcgatgatgtcccagatagaaacaatgttctacatcctgaagagtactccattgtcaatgcaccttgggaccctttatggtgatctcttcactcgtcagagcacgctcggccacagcgaagatgtatttgacattatccaagcagagatacgaactcttcatcctcacctcctcctagagatgacacctgccaaggtcgaggagatgattctgattgctgacaggaagacatatgatgagagggcagcatctgcgttctggtccactctcataacaataaagaattacattccagcttggacagagaagggggcttgtactctggattggcctagtgtgtctctagacaagaagagtgggtacgtgaaagtgatggcaggtaaagatactaccatctacattgggactgatatttgtgtgatagagaagtatcctacagtaaaatgggctccactttcataccttttaaatggagctgacaaggtggcagaaaggctgaatgtgcgttactatagttgtctttgtgatcagcttgatataccagataggataacacttgatatgcttgatgaaatcatcaaagtgggggatgattgtcttcgagagatgggcaatgacggatacaatgtaattggatcctatgaggctttgcttgcaggaataatccaaaggagggacaaccagggactaataccggacagagatttgttgtggagaactactatggaagaatttacatccagccccggcaggaagtatctagaagagtggagcacaatgtttagttcattaagccctgaacagatagcctgtgctcatggactgtataggatctgggggcacccaatagttgatattcttggagggataaagaagatgcaagaggtggcatctgtgaagaagaatcctagacagagtgtactggatgaaataaggaggcaattcaaagaaatgttttttacctcatatcataggatacacaagcactaccctctacatacaatcttgaacagcctggataattcctatatactcaactgtctaaaggataacttgacgatcaacacatcagcaatctcctataacttccaggattgggactgtgtgatggtggagaaaaacttcgaagtgcctttctcttggaacctggttcacaatttgaaagataaagctatttccccgaacaggcaggagatttaccagacgatatcaactcgtggtacaatattcgggtctcagaaccgcagagggatactgaaatcactcacgatggagactgtccaactccgagactttcttcaaggcataaatgacaacgggttggatgacatagacaaaattattggggtctatcctaaagagcgtgagctgaagataaaggctagactgttctccctcatgtcattccggctcagattgtactgtgtgtcaacagaggcattattaggggacaaaatactcaagtatttcccgcaaatcacaatgtctcttgatatgctcacgatgataaagaagatgttcaaagtgtcctcccaaactactagagaggatgattcagtcacagtcatattcaatcttgacttcataaagtggaatctccagatgagacgtaatatatgtgagcctgtcttctcacaattaggtaaactctttggcatggagaatctgttcaataggactcatgagaccttccgagatagcctgatatacctctgttcgggggaaggagtactgtcagccgatccagtctacggagtattcccggacggtacatgggcatgggcgggtgatgagtcggggaaggaagggcttcgtcagaagggttggacaattctgactgtcgttacaatcatgttgattgcaaagcgacacaatgttgatgtatcactaatgggaggaggagacaatcaagtccttggtatcaccatatcaggagtgaccagagatcttcaaggagagctgattacagaatcggcctcactcgcccgaaggacaatcaagcaatttaccgaggacttattaaccactttctccgatctggggctccctttaaaagcatctgaaacatgggtcagtgactctctcttcatgtacaacaaacacatgttttacaagggaattcctctcagatcacctctgaaagccatctcaaggatcttcccgctggcaaacgacacaattatgaccatagacaacatgatcaataatatatcatcaggggtcaaagcagcctgtatgaaggagagacacggaattccacttgtattcatgaaaacactagcatataggaaggttgccgagggagcactgatcctacatccattgacgacttgcttcaggacaccaacattgcctgaaaatggaactgtgacgcgtgacggacggaagcgatcagtgaaggtgactaggaatcagctgagatcattctttacgttatgtatggtgggatcttccactatgggactacctggtactatacatctccctgatatgatcatgaggggattccctgatccattgaccagctacctgtcattcatttcagaattggttcccaagatcatggacccaagactagcgagcatcatcgcaacatcgggtcatatgagcatcaatagaatgacagagtatgcgaaacttgtggaggatcctgtatctataaatcacgacgcccccacacatggactgaatgagatccgacagttatctcgagacttcttgatgaacacgaccctggcagccaacacccatcttaagtcccttttcaccctcctagacagacagagagaaaaagaattctacgacgcactatgttctgctgaggaactggatgtgaaggtgctgcacgaagtagccggagcatcactattcggatatacaaacggcattgcctctcgtattgaccagacaagaacggtgagagcgttaaacgagacagtcaacgttatgaaaaggctagctgatgcagaggcaagatatattgcatatctgttcgctagggacatacataagcatgacctcatcccggatcagtgttccagagtgactgcggacaggtttgtttctgcttctacctttgatatatatataataattatcattaattagtagtaatataatatttcaaatatttttttcaaaataaaagaatgtagtatatagcaattgcttttctgtagtttataagtgtgtatattttaatttataacttttctaatatatgaccaaaacatggtgatgtttaggtatcgatattattcatggaagaagaaggttttaggggtaacagtacctcacccgattgagatgtgtcaagtcacatcggcgacccaaactgcattcgaggatgctgttatatgttggagtgataatctgagcggatcgagaatctacaccacaatgggtctagggaagatatatcaaggttcctacaccaaggaaaggttcaaggccacagacattgctgccgcgtatgggaacgaggacatcctgacaaaagcagtcaggctgcagaagctaattaactggagatatgatgaagggagcagctttgctaggataattcaacttgctctcagagccatcactgatgcaagcactgacggattccatagatctaaggaggagatcaaaggggaattcgatcatagaaggggcatatcaggagatatcagcggaggaattccaaatttccttgtcacacccacctcgcatttctcgtgtaccacatcctcttggatctcacatagcaggggagggaaaaacgagaatatccatttccagagtgtactgatcaacttattatacagagccatggtctatcgaggctcattgcccggtctaccagagatggtctggtattcaaaagagagatgcagccattgcattattgatattgtggaaccgtctccagaacgaacaaccccagctttgtcagagatgccatcagcacctaacaatccttttgcattcatagagtcagtaaatgtgaagttggattatcatcaccaggttgagatagaaaaaggaatggaagagaccaacctactaaattctgagtggggaggctcatctttaacaagtgtggatgaggctggactggtcttgtttctgatgctgattggatcccgtcaagtgtcagaatcgtttttgttattgatgagggagaagatagaacctcgaccgctactgcaagcagcattgaacagagtcatccttgtcaggaaactcggattggataggttgttccctataagaaacaccaaatgcatcaatcttatactggggtctgacagaggaatcactgcatgccatgacggctttggcctggaactgcacgggggatcctgggagaaagggatagagtgtgacacctcctttctttacaatgatactcacctcaagccctgtaaattgcatgttaaccttcatcttcaaaatgtgcctctccagctggccttggcgcatgcttcctataatcctgatgtttgtgagtgtcttgagtgcagagccatcattgaagatagagtctctaagaggatggtagggcaatataagagctggagatgccagtatcatgcggtcctgtccttctctccaatcatcatgagggtccacagtgagaggataatcaaaaatgagggagctatatatgaaggggatctgcatgtcccttatgtcgaggagatcattgctctggagaactcacaaaaattggcaatagatgtgacgtcatggagactgccgctgctgatgcacgcaacatgggagtctatcctgcctcagctgtatattactatgaaggcctgcctggtttgtcttcccttggagggtgtgattgtggatgatgacctggcactgatcaatctggtgtccaaggtgttggtcgatctgaggaggaccatccaggtctatatcagagcagggacgttctccgggtcggagatcaacaataagtatgacaacatcaggctgcttccagaggatcttagggacagcattcacatcctaacaaccacagacaaaccagatcctgctgcaactgtccaactggcagcggatgacacatcgcctcttgactgtgctgtgtaccatctcatctggggcgatacctggggcatgacgaggaaggtgcacggaagaattctcattccatcctctcgtgtgacaactggattcgcaggggtcatggtgacagatcactacgcaactgtggcagtattggagcttgctggtgctgttaacctgtgggagaaaaagctcattgatcttgaacatagaaacaacctcgatgttccgttgagcatgaatcgtgtgttggcaggttctagactcgggacccgggggataagatgggctcaatggacgtcgcctgcaggcctagagatcgttgtcaggaaagtgagggcaaagctgtcatcaatctcgggaaaccctggaaatgccgccaaatggaggaggaactgccagaaggtcctcaaggccttgatgatctccctgtatgctcattcgggcgaggacatgatgaggaatgcaagcggccttgtcaaggtgaacatacatgggaccctttcctctgtcaccgctgtgtgcgacccgtcggcggagagcagagtgcagagggcacagttcctgttcctaaaagaaaaggctcccaatgggcctttcattctaaggagcaggttggagaggcgcataaatctcctctcagttgtgtccgacctgctcgggtgatcaagaagtattaagaaaaaccctcatgttcacgtggttagaggggttacacccatactacatatcaaggtctttgacaatagtgtatttcgtgagaatgccatgtttttctgggtctctgggtcggcatggcatctccacctcctcgcggtccgacctgggcatccgaaggaggacgtccactcggatggctaagggagagctcgaattcggtacgctgaaatcaccagtctctctctacaaatctatctctctctattttctccataaataatgtgtgagtagtttcccgataagggaaattagggttcttatagggtttcgctcatgtgttgagcatataagaaacccttagtatgtatttgtatttgtaaaatacttctatcaataaaatttctaattcctaaaaccaaaatccagtactaaaatccagatccccaattcactggccgtcgttttacaacgtcgtgactgggaaaaccctggcgttacccaacttaatcgcctgtttaaactatcagtgtttgacagggacctgcagcccggccgggggatccactagcagattgtcgtttcccgccttcagtttaaactatcagtgtttgacaggatatattggcgggtaaacctaagagaaaagagcgtttattagaataatcggatatttaaaagggcgtgaaaaggtttatccgttcgtccatttgtatgtgcatgccaaccacaggagatctcagtaaagcgctggctgaacccccagccggaactgaccccacaaggccctagcgtttgcaatgcaccaggtcatcattgacccaggcgtgttccaccaggccgctgcctcgcaactcttcgcaggcttcgccgacctgctcgcgccacttcttcacgcgggtggaatccgatccgcacatgaggcggaaggtttccagcttgagcgggtacggctcccggtgcgagctgaaatagtcgaacatccgtcgggccgtcggcgacagcttgcggtacttctcccatatgaatttcgtgtagtggtcgccagcaaacagcacgacgatttcctcgtcgatcaggacctggcaacgggacgttttcttgccacggtccaggacgcggaagcggtgcagcagcgacaccgattccaggtgcccaacgcggtcggacgtgaagcccatcgccgtcgcctgtaggcgcgacaggcattcctcggccttcgtgtaataccggccattgatcgaccagcccaggtcctggcaaagctcgtagaacgtgaaggtgatcggctcgccgataggggtgcgcttcgcgtactccaacacctgctgccacaccagttcgtcatcgtcggcccgcagctcgacgccggtgtaggtgatcttcacgtccttgttgacgtggaaaatgaccttgttttgcagcgcctcgcgcgggattttcttgttgcgcgtggtgaacagggcagagcgggccgtgtcgtttggcatcgctcgcatcgtgtccggccacggcgcaatatcgaacaaggaaagctgcatttccttgatctgctgcttcgtgtgtttcagcaacgcggcctgcttggcctcgctgacctgttttgccaggtcctcgccggcggtttttcgcttcttggtcgtcatagttcctcgcgtgtcgatggtcatcgacttcgccaaacctgccgcctcctgttcgagacgacgcgaacgctccacggcggccgatggcgcgggcagggcagggggagccagttgcacgctgtcgcgctcgatcttggccgtagcttgctggaccatcgagccgacggactggaaggtttcgcggggcgcacgcatgacggtgcggcttgcgatggtttcggcatcctcggcggaaaaccccgcgtcgatcagttcttgcctgtatgccttccggtcaaacgtccgattcattcaccctccttgcgggattgccccgactcacgccggggcaatgtgcccttattcctgatttgacccgcctggtgccttggtgtccagataatccaccttatcggcaatgaagtcggtcccgtagaccgtctggccgtccttctcgtacttggtattccgaatcttgccctgcacgaataccagcgaccccttgcccaaatacttgccgtgggcctcggcctgagagccaaaacacttgatgcggaagaagtcggtgcgctcctgcttgtcgccggcatcgttgcgccacatctaggtactaaaacaattcatccagtaaaatataatattttattttctcccaatcaggcttgatccccagtaagtcaaaaaatagctcgacatactgttcttccccgatatcctccctgatcgaccggacgcagaaggcaatgtcataccacttgtccgccctgccgcttctcccaagatcaataaagccacttactttgccatctttcacaaagatgttgctgtctcccaggtcgccgtgggaaaagacaagttcctcttcgggcttttccgtctttaaaaaatcatacagctcgcgcggatctttaaatggagtgtcttcttcccagttttcgcaatccacatcggccagatcgttattcagtaagtaatccaattcggctaagcggctgtctaagctattcgtatagggacaatccgatatgtcgatggagtgaaagagcctgatgcactccgcatacagctcgataatcttttcagggctttgttcatcttcatactcttccgagcaaaggacgccatcggcctcactcatgagcagattgctccagccatcatgccgttcaaagtgcaggacctttggaacaggcagctttccttccagccatagcatcatgtccttttcccgttccacatcataggtggtccctttataccggctgtccgtcatttttaaatataggttttcattttctcccaccagcttatataccttagcaggagacattccttccgtatcttttacgcagcggtatttttcgatcagttttttcaattccggtgatattctcattttagccatttattatttccttcctcttttctacagtatttaaagataccccaagaagctaattataacaagacgaactccaattcactgttccttgcattctaaaaccttaaataccagaaaacagctttttcaaagttgttttcaaagttggcgtataacatagtatcgacggagccgattttgaaaccacaattatgggtgatgctgccaactcgagagcgggccgggagggttcgagaagggggggcaccccccttcggcgtgcgcggtcacgcgcacagggcgcagccctggttaaaaacaaggtttataaatattggtttaaaagcaggttaaaagacaggttagcggtggccgaaaaacgggcggaaacccttgcaaatgctggattttctgcctgtggacagcccctcaaatgtcaataggtgcgcccctcatctgtcagcactctgcccctcaagtgtcaaggatcgcgcccctcatctgtcagtagtcgcgcccctcaagtgtcaataccgcagggcacttatccccaggcttgtccacatcatctgtgggaaactcgcgtaaaatcaggcgttttcgccgatttgcgaggctggccagctccacgtcgccggccgaaatcgagcctgcccctcatctgtcaacgccgcgccgggtgagtcggcccctcaagtgtcaacgtccgcccctcatctgtcagtgagggccaagttttccgcgaggtatccacaacgccggcggccggccgcggtgtctcgcacacggcttcgacggcgtttctggcgcgtttgcagggccatagacggccgccagcccagcggcgagggcaaccagcccggtgagctctagtggactgatgggctgcctgtatcgagtggtgattttgtgccgagctgccggtcggggagctgttggctggct

SEQ ID NO: 4 - DNA, 17850 bp, Artificial sequence
  Synthetic nucleotide
ggtggcaggatatattgtggtgtaaacaaattgacgcttagacaacttaataacacattgcggacgtttttaatgtactggggtggttttggtaccgggccccccctcgaggtcgacggtatcgataagcttgatatcgaattcctgcaggcctcctaggactagttcccaggtcaacatggtggagcacgacactctcgtctactccaagaatatcaaagatacagtctcagaagaccagagggctattgagacttttcaacaaagggtaatatcgggaaacctcctcggattccattgcccagctatctgtcacttcatcgaaaggacagtagaaaaggaagatggcttctacaaatgccatcattgcgataaaggaaaggctatcgttcaagatgcctctaccgacagtggtcccaaagatgagacttttcaacaaagggtaatatcgggaaacctcctcggattccattgcccagctatctgtcacttcatcgaaaggacagtagaaaaggaagatggcttctacaaatgccatcattgcgataaaggaaaggctatcgttcaagatgcctctaccgacagtggtcccaaagatggacccccacccacgaggaacatcgtggaaaaagaagacgttccaaccacgtcttcaaagcaagtggattgatgtgatatctccactgacgtaagggatgacgcacaatcccactatccttcgcaagacccttcctctatataaggaagttcatttcatttggagaggagagacccatatatttcataaagggtcatctggtctgtatcacatagctgattagtcctcatgatgctgggcaagaatgcttgtgggaagatccgcccgcaacctgctcaataacacatgatcaggaagtgacacaatctgcgactattaacaaaaacccaaccacctctaaccttcttcatctcatccttcataatggcaaacatcaacatcccggatgacctggtatccaagtactcggaagatgtaaaggatctcacacaaaaggcaggtagcattccaagttctaaatcattgatcccccaaaccgcattcacccctgatgaattgaggagatctttgaagttctggcaagtgaccaccaaagtggcagcaactgtagcatccgactggactaatttggcgactgcattaaccaacgggacattcagtgcgacgcatctcaggacactgtgtgagctggcattcaacctcaaggccccaactggaacaggcacaatcttcattcatgagattgctgacccgtggaaagggtgcattgatgctaatgctcccgacactgtagcgatacctgcgactgacagtgatgttaatatgtctactgtcagctcaggttctaccgcctctgggacggaagatgcagaggtcaaaatgaaggcgatctcattcctatgctgcacattgctcaggctgtctgtgaaagagccgtcacacatcatgcaagcgatcaacagcatacgacagaggttcggctccctgtacggtgtctcctcccctaccttgaatagtgttacattcaacaggagccaactttctcgcatcaagcaaggcattgaaacatatccctctgctcggggtacagtattctattacaccaggtatgccgatgtcacgcatgggtatactaccaaagagtatgggatatgcaggttcctactgttccaacaccttgaacttgaggggatgcacatctataagatgctcttagctctacttggcgagtggtcaacagttccaattgggctcttgctcacctggatcaggaacccgaggtctaagttggccataaatgagattgtcaagatagtcaaggagcttgataagccggaagtggacaagggctggaaatatgctcggatggtcaataacacattcttccttgatctatcctctagaaggaacacttacctatgtgctgtgttggcctccctgaacaagaagaacgttccgcaaggatcaggggaatatgctgaccccactaacatagctgtgatcaagaatatggaccaatctgtgaaatcacaggttgccacagatgtgaccctgattgagagaatttatgagaaatatctggtgtctgcaggtggagacgaggcaggaacagcatatgctctgtctcgcggagttaagagatcctcgcctcaaagtcaggaaggcggtcaatccggccagtctggaggaacacccatggaagtcgacggagcatctggtagaggagcagcaggtccggccccgaagaagacacgatcaggcttatagatctcagctcggtatcaatagcctacatgagaagatgtaccacgtgccatacgctcctgaacctatgtgtgatcccaatggaccaacaataatagcagagtctgtcataaccctgccgttttaatcaattatcatgtgcttcccatgttgttgtttcaatctattaagaaaaacccaacctcaggatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaaatctcagctcggtatcaatagcctacatgagaagatgtaccacgtgccatacgctcctgaacctatgtgtgatcccaatggaccaacaataatagcagagtctgtcataaccctgccgttttaatcaattatcatgtgcttcccatgttgttgtttcaatctattaagaaaaacccaacctcaggatgaatcgttactcccgtcgttctagacatcccaaccctcctgtccccaatcaggaagaacccgagcgcgacccgaatcacatagatcaagacctcgcagacttggctcaacccttggtcctaaaagaaagacacgcagtaatggcaccaactcagcccagccttagcgatgtgatcaatgaggaaagacaggctcccatcacgtttggtaatcctccagaggtcatggcgaacgctaggctatcagcgctgggttatgacaatctcacggagagggagaagaggatccttgctgtaggaatcagatgcggagaggctgccaaagactatcattcgctcactaccaccaaaaagtggattgaggatgagctgaagtctcaaatggtggctctcgcatcatcaacaaggactctcactgaggctgcatcacttcacacgacctttgcaatgctgcattctccatccattaaaagaaaggcagaagcaatgtctcacatatctcaaggcgaagagagcattgacatttctaagctgaataagactggaatggaggacatctgggtggcaatggagagtgagtcaaaagaggatgcagtagacacgtatctgagaaacattctggaagtggacccgacacaattctatgcaatagacggctggggcctctacctggacttcattcccacttggcattatatagctgcagggaagaattcggcgcaatttaagacgagttatgctgacgagattgtagagcagcgtgctgcattcgagagggtcctctctaagcgccctagggttgagatctaagcctgtatgtcactccactccccaataatgtccacgtcagcctcatgacaccctgctattaagaaaaacccaacaatggagccctctgcagagatccccctcgggaaaggcaaggaaaccgggagaagccctctgatcgcaacaaggaggaagaccgggctggtcaaagccaagataaagaccaattcatccttctctatgttcaggagagcagatactatgcgaacccatgcacagttcaccaacattactgtggggtggaagtcgctttgtcctatcactgcagaaggacaggtcaatgtagccatataccatgagagcacccgagtccctgttctcaatctatggagtcctgtgtcctctagttggaagcacctggcaacaggcagtctaggctttgttagtctgaaccactgtccatatgtggttgaaggaagaatatcgggattcgagggagaagaagcagggttggtgacacttacacttcatcttgacactggattggagagtgatgacatccagaagtgtcgtttattttctgaacatccggagttagcgggtggatcactatatttatacacttcctacacaagtagccccataccggacagcaatctccctgccttaaggactgagatcgatgacactatcgctgcaatttggaggatgagtggagggaagatgtctatcaccacaagccatgctctagggctgattgccaccttcagggttaaaatagagtctttacttggggaatacccggatgatgcattctctgctcaggttgagaatcttgtgaaggattttctccttgtttcatcgacagctgagataaccagatcttacaggacctgccttgcaaaagtgctgtcgtcgatgaccacgtatgacaagaagtactgggacaccatcataacaggtccctcctccatatgacactcgccatcgcagcagctggagctcatattcccatcatcaagcacatctgcaagaataaaaatccgctcccacccttcattcatcctcacaaccaccctccgcccttgttaagaaaaacccaacatggatgctcctatcaatccttcctctcccacccggtcttcctatgagacagccctagagaggcaatggttgatcaaggatgaaccgtcacaccatgttgacttgggatgtcacttcacattagagatcaaagcattctcacccaaaatgattgagttgctggagactggtagcctgactctaggatccgtgtatcaagggctgtacaatttgatcaaggacaaagactgcaaggggctggagctagatcgggacaccacccatgggaacaaattgcatgtcttggcggcagctttcctgtgtgtgctcaggaagtatgagaaccaggtgcggttttacaatgtcaaaaccaaggactctctgacgggcgaaatccagtatatgtctagggtacacatcggagatgataacttggggctcttcgatccagacacctacaggatgataggagggtcatttccggaagggatctaccgactgaacctccgcacacaactcacacccaggggagaagatgacggtctggaaatcataatgggtaccattctaaccgtccccgtgcgcggtcctgctgaccgacagcctctcggagtattgatggacagacaactaagacagttcccggtagggaccaacccttggtcgtccttgggggaggcgaaacctaaagggagatcgttgtcactcctgcgtggaatccttatgtaattctgcatatcacctggttgatatccaataagaatgccactgcccagcattaccccacgattaagaaaaacccaacttacacagacgcaatcatgtctctcattcacctcatctacttccccaccatcctgtctctcatcctaggtacagagggcacaccagcattgacttatggcaacctggctgacaatcagcatggaaatgaggagcaaggggatgttgatatgtatcctctatatgagtgcagtaagaagggtgcaggagtctctagcaataactggtacggagcctgccgaggagcttgctctatgaccaaaaatactactgatgtccacatggagatatattacaggaatgacagtgtcggttggatagatgtattgtctatccagactacagaaatctggaagcactcacatgtaacctggtacggagagtgtgaacacaatacaaaatatgggagctcgaacacagcacctgtctccgtcattctggagcggttggagttccttcacaatgggatgtcatcatggccatacggtcatagcattcatgttcatgacactatctcaagccctgactgtagctattttggtgatgtgtccagatccggatggagattgataatatctaaacgtgctcttgaactgaaatcagatattgcaggagagggctatattgtagaccccgatgtggggtatgtatatcctgttgcggacggaattgggaaaggtaggacctggcatgtatggaaagctagctctgttccgtcttctggctgctacttcaaatcggcaggaattactaattgtaccctactaatggatacattcctctattcatgtccaagcctcaatatagccttctcagctcgggtcgggaaacacataaagagcacgtgcgtcggagatatgaatatatcaacagacggtgtgacttatcagactctcgggagcacggatcaagggtcgatatccacacagttggtatcattatggcatcaatcacaggaagctctcatacagcaacttattttgactatcaacgaggccctaggaaaaattgagacaagctactgtgagtccacttgtgatttgaccgagatgttaatctcccagcataccgagcacccgatggtaattgagacgcctgtcgggccatggctaccatacagcatagacggtaaagtttctgttctcccttgtcaaggtggtcaggatctggtggtgatcaagcctatagagatctgcgcttctccattcatgctcaaagttaaatcactcaagtctctagacacctactggtgggtccctacagatagccacgtggctccgttcacgcaatgcagcaacagggacgatgaagaaatgatcatcaagatacaacggaagaagccattgatatttgaattctggagaggagcctataagcttgattacccgtataactctagtggaatctggctggataaccctggaggtcatattcatagaagctctaaatggttccctacgttggatagtctatcctacagttctcctataaacttgccactgatcagcaaaggtataaggaaacatattcaagccatcgtcagtaccaccgagatcacatctggaggtaacgttacatcatggacccactcgatttcgactattacagaggccatgttcgactcagctgcgatagccgcgggtcgggtcgtgatgtggtggtacacactagaagaaagcgtcaaaaggtatctgactgtagcgttcggtctagtactaactgtcatcaccatttgggctatgtcaaaattattcttccgatcgtcgccaacatatgccagggcgcccacaaatatagaatgggtaaggtcatagacatcgcagtgtccacccacctcatctccatggagcccgatccacaataaaatgccatctatgtatcaaattaatcctatatgtatattaagaaaaacccaattccaatggatccagattatcctgatcttgatcccgagtctcttgatgttctcaacagcctacaagaagggtacgatgaggaggaagaagaggacagcggagtgtcactcaatggaatgggggactaccaccttaagtcagcactcaggtcgtacacagatatgatgaagcacccaatcttcaagaaggagttctccaaggcagtgattaactttggtatatctcacgactcgatgatgtcccagatagaaacaatgttctacatcctgaagagtactccattgtcaatgcaccttgggaccctttatggtgatctcttcactcgtcagagcacgctcggccacagcgaagatgtatttgacattatccaagcagagatacgaactcttcatcctcacctcctcctagagatgacacctgccaaggtcgaggagatgattctgattgctgacaggaagacatatgatgagagggcagcatctgcgttctggtccactctcataacaataaagaattacattccagcttggacagagaagggggcttgtactctggattggcctagtgtgtctctagacaagaagagtgggtacgtgaaagtgatggcaggtaaagatactaccatctacattgggactgatatttgtgtgatagagaagtatcctacagtaaaatgggctccactttcataccttttaaatggagctgacaaggtggcagaaaggctgaatgtgcgttactatagttgtctttgtgatcagcttgatataccagataggataacacttgatatgcttgatgaaatcatcaaagtgggggatgattgtcttcgagagatgggcaatgacggatacaatgtaattggatcctatgaggctttgcttgcaggaataatccaaaggagggacaaccagggactaataccggacagagatttgttgtggagaactactatggaagaatttacatccagccccggcaggaagtatctagaagagtggagcacaatgtttagttcattaagccctgaacagatagcctgtgctcatggactgtataggatctgggggcacccaatagttgatattcttggagggataaagaagatgcaagaggtggcatctgtgaagaagaatcctagacagagtgtactggatgaaataaggaggcaattcaaagaaatgttttttacctcatatcataggatacacaagcactaccctctacatacaatcttgaacagcctggataattcctatatactcaactgtctaaaggataacttgacgatcaacacatcagcaatctcctataacttccaggattgggactgtgtgatggtggagaaaaacttcgaagtgcctttctcttggaacctggttcacaatttgaaagataaagctatttccccgaacaggcaggagatttaccagacgatatcaactcgtggtacaatattcgggtctcagaaccgcagagggatactgaaatcactcacgatggagactgtccaactccgagactttcttcaaggcataaatgacaacgggttggatgacatagacaaaattattggggtctatcctaaagagcgtgagctgaagataaaggctagactgttctccctcatgtcattccggctcagattgtactgtgtgtcaacagaggcattattaggggacaaaatactcaagtatttcccgcaaatcacaatgtctcttgatatgctcacgatgataaagaagatgttcaaagtgtcctcccaaactactagagaggatgattcagtcacagtcatattcaatcttgacttcataaagtggaatctccagatgagacgtaatatatgtgagcctgtcttctcacaattaggtaaactctttggcatggagaatctgttcaataggactcatgagaccttccgagatagcctgatatacctctgttcgggggaaggagtactgtcagccgatccagtctacggagtattcccggacggtacatgggcatgggcgggtgatgagtcggggaaggaagggcttcgtcagaagggttggacaattctgactgtcgttacaatcatgttgattgcaaagcgacacaatgttgatgtatcactaatgggaggaggagacaatcaagtccttggtatcaccatatcaggagtgaccagagatcttcaaggagagctgattacagaatcggcctcactcgcccgaaggacaatcaagcaatttaccgaggacttattaaccactttctccgatctggggctccctttaaaagcatctgaaacatgggtcagtgactctctcttcatgtacaacaaacacatgttttacaagggaattcctctcagatcacctctgaaagccatctcaaggatcttcccgctggcaaacgacacaattatgaccatagacaacatgatcaataatatatcatcaggggtcaaagcagcctgtatgaaggagagacacggaattccacttgtattcatgaaaacactagcatataggaaggttgccgagggagcactgatcctacatccattgacgacttgcttcaggacaccaacattgcctgaaaatggaactgtgacgcgtgacggacggaagcgatcagtgaaggtgactaggaatcagctgagatcattctttacgttatgtatggtgggatcttccactatgggactacctggtactatacatctccctgatatgatcatgaggggattccctgatccattgaccagctacctgtcattcatttcagaattggttcccaagatcatggacccaagactagcgagcatcatcgcaacatcgggtcatatgagcatcaatagaatgacagagtatgcgaaacttgtggaggatcctgtatctataaatcacgacgcccccacacatggactgaatgagatccgacagttatctcgagacttcttgatgaacacgaccctggcagccaacacccatcttaagtcccttttcaccctcctagacagacagagagaaaaagaattctacgacgcactatgttctgctgaggaactggatgtgaaggtgctgcacgaagtagccggagcatcactattcggatatacaaacggcattgcctctcgtattgaccagacaagaacggtgagagcgttaaacgagacagtcaacgttatgaaaaggctagctgatgcagaggcaagatatattgcatatctgttcgctagggacatacataagcatgacctcatcccggatcagtgttccagagtgactgcggacaggtttgtttctgcttctacctttgatatatatataataattatcattaattagtagtaatataatatttcaaatatttttttcaaaataaaagaatgtagtatatagcaattgcttttctgtagtttataagtgtgtatattttaatttataacttttctaatatatgaccaaaacatggtgatgtttaggtatcgatattattcatggaagaagaaggttttaggggtaacagtacctcacccgattgagatgtgtcaagtcacatcggcgacccaaactgcattcgaggatgctgttatatgttggagtgataatctgagcggatcgagaatctacaccacaatgggtctagggaagatatatcaaggttcctacaccaaggaaaggttcaaggccacagacattgctgccgcgtatgggaacgaggacatcctgacaaaagcagtcaggctgcagaagctaattaactggagatatgatgaagggagcagctttgctaggataattcaacttgctctcagagccatcactgatgcaagcactgacggattccatagatctaaggaggagatcaaaggggaattcgatcatagaaggggcatatcaggagatatcagcggaggaattccaaatttccttgtcacacccacctcgcatttctcgtgtaccacatcctcttggatctcacatagcaggggagggaaaaacgagaatatccatttccagagtgtactgatcaacttattatacagagccatggtctatcgaggctcattgcccggtctaccagagatggtctggtattcaaaagagagatgcagccattgcattattgatattgtggaaccgtctccagaacgaacaaccccagctttgtcagagatgccatcagcacctaacaatccttttgcattcatagagtcagtaaatgtgaagttggattatcatcaccaggttgagatagaaaaaggaatggaagagaccaacctactaaattctgagtggggaggctcatctttaacaagtgtggatgaggctggactggtcttgtttctgatgctgattggatcccgtcaagtgtcagaatcgtttttgttattgatgagggagaagatagaacctcgaccgctactgcaagcagcattgaacagagtcatccttgtcaggaaactcggattggataggttgttccctataagaaacaccaaatgcatcaatcttatactggggtctgacagaggaatcactgcatgccatgacggctttggcctggaactgcacgggggatcctgggagaaagggatagagtgtgacacctcctttctttacaatgatactcacctcaagccctgtaaattgcatgttaaccttcatcttcaaaatgtgcctctccagctggccttggcgcatgcttcctataatcctgatgtttgtgagtgtcttgagtgcagagccatcattgaagatagagtctctaagaggatggtagggcaatataagagctggagatgccagtatcatgcggtcctgtccttctctccaatcatcatgagggtccacagtgagaggataatcaaaaatgagggagctatatatgaaggggatctgcatgtcccttatgtcgaggagatcattgctctggagaactcacaaaaattggcaatagatgtgacgtcatggagactgccgctgctgatgcacgcaacatgggagtctatcctgcctcagctgtatattactatgaaggcctgcctggtttgtcttcccttggagggtgtgattgtggatgatgacctggcactgatcaatctggtgtccaaggtgttggtcgatctgaggaggaccatccaggtctatatcagagcagggacgttctccgggtcggagatcaacaataagtatgacaacatcaggctgcttccagaggatcttagggacagcattcacatcctaacaaccacagacaaaccagatcctgctgcaactgtccaactggcagcggatgacacatcgcctcttgactgtgctgtgtaccatctcatctggggcgatacctggggcatgacgaggaaggtgcacggaagaattctcattccatcctctcgtgtgacaactggattcgcaggggtcatggtgacagatcactacgcaactgtggcagtattggagcttgctggtgctgttaacctgtgggagaaaaagctcattgatcttgaacatagaaacaacctcgatgttccgttgagcatgaatcgtgtgttggcaggttctagactcgggacccgggggataagatgggctcaatggacgtcgcctgcaggcctagagatcgttgtcaggaaagtgagggcaaagctgtcatcaatctcgggaaaccctggaaatgccgccaaatggaggaggaactgccagaaggtcctcaaggccttgatgatctccctgtatgctcattcgggcgaggacatgatgaggaatgcaagcggccttgtcaaggtgaacatacatgggaccctttcctctgtcaccgctgtgtgcgacccgtcggcggagagcagagtgcagagggcacagttcctgttcctaaaagaaaaggctcccaatgggcctttcattctaaggagcaggttggagaggcgcataaatctcctctcagttgtgtccgacctgctcgggtgatcaagaagtattaagaaaaaccctcatgttcacgtggttagaggggttacacccatactacatatcaaggtctttgacaatagtgtatttcgtgagaatgccatgtttttctgggtctctgggtcggcatggcatctccacctcctcgcggtccgacctgggcatccgaaggaggacgtccactcggatggctaagggagagctcgaattcggtacgctgaaatcaccagtctctctctacaaatctatctctctctattttctccataaataatgtgtgagtagtttcccgataagggaaattagggttcttatagggtttcgctcatgtgttgagcatataagaaacccttagtatgtatttgtatttgtaaaatacttctatcaataaaatttctaattcctaaaaccaaaatccagtactaaaatccagatccccaattcactggccgtcgttttacaacgtcgtgactgggaaaaccctggcgttacccaacttaatcgcctgtttaaactatcagtgtttgacagggacctgcagcccggccgggggatccactagcagattgtcgtttcccgccttcagtttaaactatcagtgtttgacaggatatattggcgggtaaacctaagagaaaagagcgtttattagaataatcggatatttaaaagggcgtgaaaaggtttatccgttcgtccatttgtatgtgcatgccaaccacaggagatctcagtaaagcgctggctgaacccccagccggaactgaccccacaaggccctagcgtttgcaatgcaccaggtcatcattgacccaggcgtgttccaccaggccgctgcctcgcaactcttcgcaggcttcgccgacctgctcgcgccacttcttcacgcgggtggaatccgatccgcacatgaggcggaaggtttccagcttgagcgggtacggctcccggtgcgagctgaaatagtcgaacatccgtcgggccgtcggcgacagcttgcggtacttctcccatatgaatttcgtgtagtggtcgccagcaaacagcacgacgatttcctcgtcgatcaggacctggcaacgggacgttttcttgccacggtccaggacgcggaagcggtgcagcagcgacaccgattccaggtgcccaacgcggtcggacgtgaagcccatcgccgtcgcctgtaggcgcgacaggcattcctcggccttcgtgtaataccggccattgatcgaccagcccaggtcctggcaaagctcgtagaacgtgaaggtgatcggctcgccgataggggtgcgcttcgcgtactccaacacctgctgccacaccagttcgtcatcgtcggcccgcagctcgacgccggtgtaggtgatcttcacgtccttgttgacgtggaaaatgaccttgttttgcagcgcctcgcgcgggattttcttgttgcgcgtggtgaacagggcagagcgggccgtgtcgtttggcatcgctcgcatcgtgtccggccacggcgcaatatcgaacaaggaaagctgcatttccttgatctgctgcttcgtgtgtttcagcaacgcggcctgcttggcctcgctgacctgttttgccaggtcctcgccggcggtttttcgcttcttggtcgtcatagttcctcgcgtgtcgatggtcatcgacttcgccaaacctgccgcctcctgttcgagacgacgcgaacgctccacggcggccgatggcgcgggcagggcagggggagccagttgcacgctgtcgcgctcgatcttggccgtagcttgctggaccatcgagccgacggactggaaggtttcgcggggcgcacgcatgacggtgcggcttgcgatggtttcggcatcctcggcggaaaaccccgcgtcgatcagttcttgcctgtatgccttccggtcaaacgtccgattcattcaccctccttgcgggattgccccgactcacgccggggcaatgtgcccttattcctgatttgacccgcctggtgccttggtgtccagataatccaccttatcggcaatgaagtcggtcccgtagaccgtctggccgtccttctcgtacttggtattccgaatcttgccctgcacgaataccagcgaccccttgcccaaatacttgccgtgggcctcggcctgagagccaaaacacttgatgcggaagaagtcggtgcgctcctgcttgtcgccggcatcgttgcgccacatctaggtactaaaacaattcatccagtaaaatataatattttattttctcccaatcaggcttgatccccagtaagtcaaaaaatagctcgacatactgttcttccccgatatcctccctgatcgaccggacgcagaaggcaatgtcataccacttgtccgccctgccgcttctcccaagatcaataaagccacttactttgccatctttcacaaagatgttgctgtctcccaggtcgccgtgggaaaagacaagttcctcttcgggcttttccgtctttaaaaaatcatacagctcgcgcggatctttaaatggagtgtcttcttcccagttttcgcaatccacatcggccagatcgttattcagtaagtaatccaattcggctaagcggctgtctaagctattcgtatagggacaatccgatatgtcgatggagtgaaagagcctgatgcactccgcatacagctcgataatcttttcagggctttgttcatcttcatactcttccgagcaaaggacgccatcggcctcactcatgagcagattgctccagccatcatgccgttcaaagtgcaggacctttggaacaggcagctttccttccagccatagcatcatgtccttttcccgttccacatcataggtggtccctttataccggctgtccgtcatttttaaatataggttttcattttctcccaccagcttatataccttagcaggagacattccttccgtatcttttacgcagcggtatttttcgatcagttttttcaattccggtgatattctcattttagccatttattatttccttcctcttttctacagtatttaaagataccccaagaagctaattataacaagacgaactccaattcactgttccttgcattctaaaaccttaaataccagaaaacagctttttcaaagttgttttcaaagttggcgtataacatagtatcgacggagccgattttgaaaccacaattatgggtgatgctgccaactcgagagcgggccgggagggttcgagaagggggggcaccccccttcggcgtgcgcggtcacgcgcacagggcgcagccctggttaaaaacaaggtttataaatattggtttaaaagcaggttaaaagacaggttagcggtggccgaaaaacgggcggaaacccttgcaaatgctggattttctgcctgtggacagcccctcaaatgtcaataggtgcgcccctcatctgtcagcactctgcccctcaagtgtcaaggatcgcgcccctcatctgtcagtagtcgcgcccctcaagtgtcaataccgcagggcacttatccccaggcttgtccacatcatctgtgggaaactcgcgtaaaatcaggcgttttcgccgatttgcgaggctggccagctccacgtcgccggccgaaatcgagcctgcccctcatctgtcaacgccgcgccgggtgagtcggcccctcaagtgtcaacgtccgcccctcatctgtcagtgagggccaagttttccgcgaggtatccacaacgccggcggccggccgcggtgtctcgcacacggcttcgacggcgtttctggcgcgtttgcagggccatagacggccgccagcccagcggcgagggcaaccagcccggtgagctctagtggactgatgggctgcctgtatcgagtggtgattttgtgccgagctgccggtcggggagctgttggctggct

SEQ ID NO: 5 - DNA, 19034 bp, Artificial sequence
  Synthetic nucleotide
ggtggcaggatatattgtggtgtaaacaaattgacgcttagacaacttaataacacattgcggacgtttttaatgtactggggtggttttggtaccgggccccccctcgaggtcgacggtatcgataagcttgatatcgaattcctgcaggcctcctaggactagttcccaggtcaacatggtggagcacgacactctcgtctactccaagaatatcaaagatacagtctcagaagaccagagggctattgagacttttcaacaaagggtaatatcgggaaacctcctcggattccattgcccagctatctgtcacttcatcgaaaggacagtagaaaaggaagatggcttctacaaatgccatcattgcgataaaggaaaggctatcgttcaagatgcctctaccgacagtggtcccaaagatgagacttttcaacaaagggtaatatcgggaaacctcctcggattccattgcccagctatctgtcacttcatcgaaaggacagtagaaaaggaagatggcttctacaaatgccatcattgcgataaaggaaaggctatcgttcaagatgcctctaccgacagtggtcccaaagatggacccccacccacgaggaacatcgtggaaaaagaagacgttccaaccacgtcttcaaagcaagtggattgatgtgatatctccactgacgtaagggatgacgcacaatcccactatccttcgcaagacccttcctctatataaggaagttcatttcatttggagaggagagacccatatatttcataaagggtcatctggtctgtatcacatagctgattagtcctcatgatgctgggcaagaatgcttgtgggaagatccgcccgcaacctgctcaataacacatgatcaggaagtgacacaatctgcgactattaacaaaaacccaaccacctctaaccttcttcatctcatccttcataatggcaaacatcaacatcccggatgacctggtatccaagtactcggaagatgtaaaggatctcacacaaaaggcaggtagcattccaagttctaaatcattgatcccccaaaccgcattcacccctgatgaattgaggagatctttgaagttctggcaagtgaccaccaaagtggcagcaactgtagcatccgactggactaatttggcgactgcattaaccaacgggacattcagtgcgacgcatctcaggacactgtgtgagctggcattcaacctcaaggccccaactggaacaggcacaatcttcattcatgagattgctgacccgtggaaagggtgcattgatgctaatgctcccgacactgtagcgatacctgcgactgacagtgatgttaatatgtctactgtcagctcaggttctaccgcctctgggacggaagatgcagaggtcaaaatgaaggcgatctcattcctatgctgcacattgctcaggctgtctgtgaaagagccgtcacacatcatgcaagcgatcaacagcatacgacagaggttcggctccctgtacggtgtctcctcccctaccttgaatagtgttacattcaacaggagccaactttctcgcatcaagcaaggcattgaaacatatccctctgctcggggtacagtattctattacaccaggtatgccgatgtcacgcatgggtatactaccaaagagtatgggatatgcaggttcctactgttccaacaccttgaacttgaggggatgcacatctataagatgctcttagctctacttggcgagtggtcaacagttccaattgggctcttgctcacctggatcaggaacccgaggtctaagttggccataaatgagattgtcaagatagtcaaggagcttgataagccggaagtggacaagggctggaaatatgctcggatggtcaataacacattcttccttgatctatcctctagaaggaacacttacctatgtgctgtgttggcctccctgaacaagaagaacgttccgcaaggatcaggggaatatgctgaccccactaacatagctgtgatcaagaatatggaccaatctgtgaaatcacaggttgccacagatgtgaccctgattgagagaatttatgagaaatatctggtgtctgcaggtggagacgaggcaggaacagcatatgctctgtctcgcggagttaagagatcctcgcctcaaagtcaggaaggcggtcaatccggccagtctggaggaacacccatggaagtcgacggagcatctggtagaggagcagcaggtccggccccgaagaagacacgatcaggcttatagatctcagctcggtatcaatagcctacatgagaagatgtaccacgtgccatacgctcctgaacctatgtgtgatcccaatggaccaacaataatagcagagtctgtcataaccctgccgttttaatcaattatcatgtgcttcccatgttgttgtttcaatctattaagaaaaacccaacctcaggatggactacaaagaccatgacggtgattataaagatcatgatatcgattacaaggatgacgatgacaagatggcaattttagatttaaaatctttagtactcaatgcaataaattattggggtcctaaaaataataatggcatacagggtggtgattttggttaccctatatcagaaaaacaaatagatacgtctattataacttttactcatcctcgtttaattccatatgatttaacaattcctcaaaatttagaaactatttttactacaactcaagtattaacaaataatacagatttacaacaaagtcaaactgtttcttttgctaaaaaaacaacgacaacaacttcaacttcaactacaaatggttggacagaaggtgggaaaatttcagatacattagaagaaaaagtaagtgtatctattccttttattggagagggaggaggaaaaaacagtacaactatagaagctaattttgcacataactctagtactactacttttcaacaggcttcaactgatatagagtggaatatttcacaaccagtattggttcccccaagtaaacaagttgtagcaacattagttattatgggaggtaattttactattcctatggatttgatgactactatagattctacagaacattatagccattatagtggttatccaatattaacatggatatcgagccccgataatagttatagtggtccatttatgagttggtattttgcaaattggcccaatttaccatcggggtttggtcctttaaattcagataatacggtcacttatacaggttctgttgtaagtcaagtatcagctggtgtatatgccactgtacgatttgatcaatatgatatacacaatttaaggacaattgaaaaaacttggtatgcacgacatgcaactcttcataatggaaagaaaatatctataaataatgttactgaaatggcaccaacaagtccaataaaaacaaattaaatctcagctcggtatcaatagcctacatgagaagatgtaccacgtgccatacgctcctgaacctatgtgtgatcccaatggaccaacaataatagcagagtctgtcataaccctgccgttttaatcaattatcatgtgcttcccatgttgttgtttcaatctattaagaaaaacccaacctcagcatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaaatctcagctcggtatcaatagcctacatgagaagatgtaccacgtgccatacgctcctgaacctatgtgtgatcccaatggaccaacaataatagcagagtctgtcataaccctgccgttttaatcaattatcatgtgcttcccatgttgttgtttcaatctattaagaaaaacccaacctcaggatgaatcgttactcccgtcgttctagacatcccaaccctcctgtccccaatcaggaagaacccgagcgcgacccgaatcacatagatcaagacctcgcagacttggctcaacccttggtcctaaaagaaagacacgcagtaatggcaccaactcagcccagccttagcgatgtgatcaatgaggaaagacaggctcccatcacgtttggtaatcctccagaggtcatggcgaacgctaggctatcagcgctgggttatgacaatctcacggagagggagaagaggatccttgctgtaggaatcagatgcggagaggctgccaaagactatcattcgctcactaccaccaaaaagtggattgaggatgagctgaagtctcaaatggtggctctcgcatcatcaacaaggactctcactgaggctgcatcacttcacacgacctttgcaatgctgcattctccatccattaaaagaaaggcagaagcaatgtctcacatatctcaaggcgaagagagcattgacatttctaagctgaataagactggaatggaggacatctgggtggcaatggagagtgagtcaaaagaggatgcagtagacacgtatctgagaaacattctggaagtggacccgacacaattctatgcaatagacggctggggcctctacctggacttcattcccacttggcattatatagctgcagggaagaattcggcgcaatttaagacgagttatgctgacgagattgtagagcagcgtgctgcattcgagagggtcctctctaagcgccctagggttgagatctaagcctgtatgtcactccactccccaataatgtccacgtcagcctcatgacaccctgctattaagaaaaacccaacaatggagccctctgcagagatccccctcgggaaaggcaaggaaaccgggagaagccctctgatcgcaacaaggaggaagaccgggctggtcaaagccaagataaagaccaattcatccttctctatgttcaggagagcagatactatgcgaacccatgcacagttcaccaacattactgtggggtggaagtcgctttgtcctatcactgcagaaggacaggtcaatgtagccatataccatgagagcacccgagtccctgttctcaatctatggagtcctgtgtcctctagttggaagcacctggcaacaggcagtctaggctttgttagtctgaaccactgtccatatgtggttgaaggaagaatatcgggattcgagggagaagaagcagggttggtgacacttacacttcatcttgacactggattggagagtgatgacatccagaagtgtcgtttattttctgaacatccggagttagcgggtggatcactatatttatacacttcctacacaagtagccccataccggacagcaatctccctgccttaaggactgagatcgatgacactatcgctgcaatttggaggatgagtggagggaagatgtctatcaccacaagccatgctctagggctgattgccaccttcagggttaaaatagagtctttacttggggaatacccggatgatgcattctctgctcaggttgagaatcttgtgaaggattttctccttgtttcatcgacagctgagataaccagatcttacaggacctgccttgcaaaagtgctgtcgtcgatgaccacgtatgacaagaagtactgggacaccatcataacaggtccctcctccatatgacactcgccatcgcagcagctggagctcatattcccatcatcaagcacatctgcaagaataaaaatccgctcccacccttcattcatcctcacaaccaccctccgcccttgttaagaaaaacccaacatggatgctcctatcaatccttcctctcccacccggtcttcctatgagacagccctagagaggcaatggttgatcaaggatgaaccgtcacaccatgttgacttgggatgtcacttcacattagagatcaaagcattctcacccaaaatgattgagttgctggagactggtagcctgactctaggatccgtgtatcaagggctgtacaatttgatcaaggacaaagactgcaaggggctggagctagatcgggacaccacccatgggaacaaattgcatgtcttggcggcagctttcctgtgtgtgctcaggaagtatgagaaccaggtgcggttttacaatgtcaaaaccaaggactctctgacgggcgaaatccagtatatgtctagggtacacatcggagatgataacttggggctcttcgatccagacacctacaggatgataggagggtcatttccggaagggatctaccgactgaacctccgcacacaactcacacccaggggagaagatgacggtctggaaatcataatgggtaccattctaaccgtccccgtgcgcggtcctgctgaccgacagcctctcggagtattgatggacagacaactaagacagttcccggtagggaccaacccttggtcgtccttgggggaggcgaaacctaaagggagatcgttgtcactcctgcgtggaatccttatgtaattctgcatatcacctggttgatatccaataagaatgccactgcccagcattaccccacgattaagaaaaacccaacttacacagacgcaatcatgtctctcattcacctcatctacttccccaccatcctgtctctcatcctaggtacagagggcacaccagcattgacttatggcaacctggctgacaatcagcatggaaatgaggagcaaggggatgttgatatgtatcctctatatgagtgcagtaagaagggtgcaggagtctctagcaataactggtacggagcctgccgaggagcttgctctatgaccaaaaatactactgatgtccacatggagatatattacaggaatgacagtgtcggttggatagatgtattgtctatccagactacagaaatctggaagcactcacatgtaacctggtacggagagtgtgaacacaatacaaaatatgggagctcgaacacagcacctgtctccgtcattctggagcggttggagttccttcacaatgggatgtcatcatggccatacggtcatagcattcatgttcatgacactatctcaagccctgactgtagctattttggtgatgtgtccagatccggatggagattgataatatctaaacgtgctcttgaactgaaatcagatattgcaggagagggctatattgtagaccccgatgtggggtatgtatatcctgttgcggacggaattgggaaaggtaggacctggcatgtatggaaagctagctctgttccgtcttctggctgctacttcaaatcggcaggaattactaattgtaccctactaatggatacattcctctattcatgtccaagcctcaatatagccttctcagctcgggtcgggaaacacataaagagcacgtgcgtcggagatatgaatatatcaacagacggtgtgacttatcagactctcgggagcacggatcaagggtcgatatccacacagttggtatcattatggcatcaatcacaggaagctctcatacagcaacttattttgactatcaacgaggccctaggaaaaattgagacaagctactgtgagtccacttgtgatttgaccgagatgttaatctcccagcataccgagcacccgatggtaattgagacgcctgtcgggccatggctaccatacagcatagacggtaaagtttctgttctcccttgtcaaggtggtcaggatctggtggtgatcaagcctatagagatctgcgcttctccattcatgctcaaagttaaatcactcaagtctctagacacctactggtgggtccctacagatagccacgtggctccgttcacgcaatgcagcaacagggacgatgaagaaatgatcatcaagatacaacggaagaagccattgatatttgaattctggagaggagcctataagcttgattacccgtataactctagtggaatctggctggataaccctggaggtcatattcatagaagctctaaatggttccctacgttggatagtctatcctacagttctcctataaacttgccactgatcagcaaaggtataaggaaacatattcaagccatcgtcagtaccaccgagatcacatctggaggtaacgttacatcatggacccactcgatttcgactattacagaggccatgttcgactcagctgcgatagccgcgggtcgggtcgtgatgtggtggtacacactagaagaaagcgtcaaaaggtatctgactgtagcgttcggtctagtactaactgtcatcaccatttgggctatgtcaaaattattcttccgatcgtcgccaacatatgccagggcgcccacaaatatagaatgggtaaggtcatagacatcgcagtgtccacccacctcatctccatggagcccgatccacaataaaatgccatctatgtatcaaattaatcctatatgtatattaagaaaaacccaattccaatggatccagattatcctgatcttgatcccgagtctcttgatgttctcaacagcctacaagaagggtacgatgaggaggaagaagaggacagcggagtgtcactcaatggaatgggggactaccaccttaagtcagcactcaggtcgtacacagatatgatgaagcacccaatcttcaagaaggagttctccaaggcagtgattaactttggtatatctcacgactcgatgatgtcccagatagaaacaatgttctacatcctgaagagtactccattgtcaatgcaccttgggaccctttatggtgatctcttcactcgtcagagcacgctcggccacagcgaagatgtatttgacattatccaagcagagatacgaactcttcatcctcacctcctcctagagatgacacctgccaaggtcgaggagatgattctgattgctgacaggaagacatatgatgagagggcagcatctgcgttctggtccactctcataacaataaagaattacattccagcttggacagagaagggggcttgtactctggattggcctagtgtgtctctagacaagaagagtgggtacgtgaaagtgatggcaggtaaagatactaccatctacattgggactgatatttgtgtgatagagaagtatcctacagtaaaatgggctccactttcataccttttaaatggagctgacaaggtggcagaaaggctgaatgtgcgttactatagttgtctttgtgatcagcttgatataccagataggataacacttgatatgcttgatgaaatcatcaaagtgggggatgattgtcttcgagagatgggcaatgacggatacaatgtaattggatcctatgaggctttgcttgcaggaataatccaaaggagggacaaccagggactaataccggacagagatttgttgtggagaactactatggaagaatttacatccagccccggcaggaagtatctagaagagtggagcacaatgtttagttcattaagccctgaacagatagcctgtgctcatggactgtataggatctgggggcacccaatagttgatattcttggagggataaagaagatgcaagaggtggcatctgtgaagaagaatcctagacagagtgtactggatgaaataaggaggcaattcaaagaaatgttttttacctcatatcataggatacacaagcactaccctctacatacaatcttgaacagcctggataattcctatatactcaactgtctaaaggataacttgacgatcaacacatcagcaatctcctataacttccaggattgggactgtgtgatggtggagaaaaacttcgaagtgcctttctcttggaacctggttcacaatttgaaagataaagctatttccccgaacaggcaggagatttaccagacgatatcaactcgtggtacaatattcgggtctcagaaccgcagagggatactgaaatcactcacgatggagactgtccaactccgagactttcttcaaggcataaatgacaacgggttggatgacatagacaaaattattggggtctatcctaaagagcgtgagctgaagataaaggctagactgttctccctcatgtcattccggctcagattgtactgtgtgtcaacagaggcattattaggggacaaaatactcaagtatttcccgcaaatcacaatgtctcttgatatgctcacgatgataaagaagatgttcaaagtgtcctcccaaactactagagaggatgattcagtcacagtcatattcaatcttgacttcataaagtggaatctccagatgagacgtaatatatgtgagcctgtcttctcacaattaggtaaactctttggcatggagaatctgttcaataggactcatgagaccttccgagatagcctgatatacctctgttcgggggaaggagtactgtcagccgatccagtctacggagtattcccggacggtacatgggcatgggcgggtgatgagtcggggaaggaagggcttcgtcagaagggttggacaattctgactgtcgttacaatcatgttgattgcaaagcgacacaatgttgatgtatcactaatgggaggaggagacaatcaagtccttggtatcaccatatcaggagtgaccagagatcttcaaggagagctgattacagaatcggcctcactcgcccgaaggacaatcaagcaatttaccgaggacttattaaccactttctccgatctggggctccctttaaaagcatctgaaacatgggtcagtgactctctcttcatgtacaacaaacacatgttttacaagggaattcctctcagatcacctctgaaagccatctcaaggatcttcccgctggcaaacgacacaattatgaccatagacaacatgatcaataatatatcatcaggggtcaaagcagcctgtatgaaggagagacacggaattccacttgtattcatgaaaacactagcatataggaaggttgccgagggagcactgatcctacatccattgacgacttgcttcaggacaccaacattgcctgaaaatggaactgtgacgcgtgacggacggaagcgatcagtgaaggtgactaggaatcagctgagatcattctttacgttatgtatggtgggatcttccactatgggactacctggtactatacatctccctgatatgatcatgaggggattccctgatccattgaccagctacctgtcattcatttcagaattggttcccaagatcatggacccaagactagcgagcatcatcgcaacatcgggtcatatgagcatcaatagaatgacagagtatgcgaaacttgtggaggatcctgtatctataaatcacgacgcccccacacatggactgaatgagatccgacagttatctcgagacttcttgatgaacacgaccctggcagccaacacccatcttaagtcccttttcaccctcctagacagacagagagaaaaagaattctacgacgcactatgttctgctgaggaactggatgtgaaggtgctgcacgaagtagccggagcatcactattcggatatacaaacggcattgcctctcgtattgaccagacaagaacggtgagagcgttaaacgagacagtcaacgttatgaaaaggctagctgatgcagaggcaagatatattgcatatctgttcgctagggacatacataagcatgacctcatcccggatcagtgttccagagtgactgcggacaggtttgtttctgcttctacctttgatatatatataataattatcattaattagtagtaatataatatttcaaatatttttttcaaaataaaagaatgtagtatatagcaattgcttttctgtagtttataagtgtgtatattttaatttataacttttctaatatatgaccaaaacatggtgatgtttaggtatcgatattattcatggaagaagaaggttttaggggtaacagtacctcacccgattgagatgtgtcaagtcacatcggcgacccaaactgcattcgaggatgctgttatatgttggagtgataatctgagcggatcgagaatctacaccacaatgggtctagggaagatatatcaaggttcctacaccaaggaaaggttcaaggccacagacattgctgccgcgtatgggaacgaggacatcctgacaaaagcagtcaggctgcagaagctaattaactggagatatgatgaagggagcagctttgctaggataattcaacttgctctcagagccatcactgatgcaagcactgacggattccatagatctaaggaggagatcaaaggggaattcgatcatagaaggggcatatcaggagatatcagcggaggaattccaaatttccttgtcacacccacctcgcatttctcgtgtaccacatcctcttggatctcacatagcaggggagggaaaaacgagaatatccatttccagagtgtactgatcaacttattatacagagccatggtctatcgaggctcattgcccggtctaccagagatggtctggtattcaaaagagagatgcagccattgcattattgatattgtggaaccgtctccagaacgaacaaccccagctttgtcagagatgccatcagcacctaacaatccttttgcattcatagagtcagtaaatgtgaagttggattatcatcaccaggttgagatagaaaaaggaatggaagagaccaacctactaaattctgagtggggaggctcatctttaacaagtgtggatgaggctggactggtcttgtttctgatgctgattggatcccgtcaagtgtcagaatcgtttttgttattgatgagggagaagatagaacctcgaccgctactgcaagcagcattgaacagagtcatccttgtcaggaaactcggattggataggttgttccctataagaaacaccaaatgcatcaatcttatactggggtctgacagaggaatcactgcatgccatgacggctttggcctggaactgcacgggggatcctgggagaaagggatagagtgtgacacctcctttctttacaatgatactcacctcaagccctgtaaattgcatgttaaccttcatcttcaaaatgtgcctctccagctggccttggcgcatgcttcctataatcctgatgtttgtgagtgtcttgagtgcagagccatcattgaagatagagtctctaagaggatggtagggcaatataagagctggagatgccagtatcatgcggtcctgtccttctctccaatcatcatgagggtccacagtgagaggataatcaaaaatgagggagctatatatgaaggggatctgcatgtcccttatgtcgaggagatcattgctctggagaactcacaaaaattggcaatagatgtgacgtcatggagactgccgctgctgatgcacgcaacatgggagtctatcctgcctcagctgtatattactatgaaggcctgcctggtttgtcttcccttggagggtgtgattgtggatgatgacctggcactgatcaatctggtgtccaaggtgttggtcgatctgaggaggaccatccaggtctatatcagagcagggacgttctccgggtcggagatcaacaataagtatgacaacatcaggctgcttccagaggatcttagggacagcattcacatcctaacaaccacagacaaaccagatcctgctgcaactgtccaactggcagcggatgacacatcgcctcttgactgtgctgtgtaccatctcatctggggcgatacctggggcatgacgaggaaggtgcacggaagaattctcattccatcctctcgtgtgacaactggattcgcaggggtcatggtgacagatcactacgcaactgtggcagtattggagcttgctggtgctgttaacctgtgggagaaaaagctcattgatcttgaacatagaaacaacctcgatgttccgttgagcatgaatcgtgtgttggcaggttctagactcgggacccgggggataagatgggctcaatggacgtcgcctgcaggcctagagatcgttgtcaggaaagtgagggcaaagctgtcatcaatctcgggaaaccctggaaatgccgccaaatggaggaggaactgccagaaggtcctcaaggccttgatgatctccctgtatgctcattcgggcgaggacatgatgaggaatgcaagcggccttgtcaaggtgaacatacatgggaccctttcctctgtcaccgctgtgtgcgacccgtcggcggagagcagagtgcagagggcacagttcctgttcctaaaagaaaaggctcccaatgggcctttcattctaaggagcaggttggagaggcgcataaatctcctctcagttgtgtccgacctgctcgggtgatcaagaagtattaagaaaaaccctcatgttcacgtggttagaggggttacacccatactacatatcaaggtctttgacaatagtgtatttcgtgagaatgccatgtttttctgggtctctgggtcggcatggcatctccacctcctcgcggtccgacctgggcatccgaaggaggacgtccactcggatggctaagggagagctcgaattcggtacgctgaaatcaccagtctctctctacaaatctatctctctctattttctccataaataatgtgtgagtagtttcccgataagggaaattagggttcttatagggtttcgctcatgtgttgagcatataagaaacccttagtatgtatttgtatttgtaaaatacttctatcaataaaatttctaattcctaaaaccaaaatccagtactaaaatccagatccccaattcactggccgtcgttttacaacgtcgtgactgggaaaaccctggcgttacccaacttaatcgcctgtttaaactatcagtgtttgacagggacctgcagcccggccgggggatccactagcagattgtcgtttcccgccttcagtttaaactatcagtgtttgacaggatatattggcgggtaaacctaagagaaaagagcgtttattagaataatcggatatttaaaagggcgtgaaaaggtttatccgttcgtccatttgtatgtgcatgccaaccacaggagatctcagtaaagcgctggctgaacccccagccggaactgaccccacaaggccctagcgtttgcaatgcaccaggtcatcattgacccaggcgtgttccaccaggccgctgcctcgcaactcttcgcaggcttcgccgacctgctcgcgccacttcttcacgcgggtggaatccgatccgcacatgaggcggaaggtttccagcttgagcgggtacggctcccggtgcgagctgaaatagtcgaacatccgtcgggccgtcggcgacagcttgcggtacttctcccatatgaatttcgtgtagtggtcgccagcaaacagcacgacgatttcctcgtcgatcaggacctggcaacgggacgttttcttgccacggtccaggacgcggaagcggtgcagcagcgacaccgattccaggtgcccaacgcggtcggacgtgaagcccatcgccgtcgcctgtaggcgcgacaggcattcctcggccttcgtgtaataccggccattgatcgaccagcccaggtcctggcaaagctcgtagaacgtgaaggtgatcggctcgccgataggggtgcgcttcgcgtactccaacacctgctgccacaccagttcgtcatcgtcggcccgcagctcgacgccggtgtaggtgatcttcacgtccttgttgacgtggaaaatgaccttgttttgcagcgcctcgcgcgggattttcttgttgcgcgtggtgaacagggcagagcgggccgtgtcgtttggcatcgctcgcatcgtgtccggccacggcgcaatatcgaacaaggaaagctgcatttccttgatctgctgcttcgtgtgtttcagcaacgcggcctgcttggcctcgctgacctgttttgccaggtcctcgccggcggtttttcgcttcttggtcgtcatagttcctcgcgtgtcgatggtcatcgacttcgccaaacctgccgcctcctgttcgagacgacgcgaacgctccacggcggccgatggcgcgggcagggcagggggagccagttgcacgctgtcgcgctcgatcttggccgtagcttgctggaccatcgagccgacggactggaaggtttcgcggggcgcacgcatgacggtgcggcttgcgatggtttcggcatcctcggcggaaaaccccgcgtcgatcagttcttgcctgtatgccttccggtcaaacgtccgattcattcaccctccttgcgggattgccccgactcacgccggggcaatgtgcccttattcctgatttgacccgcctggtgccttggtgtccagataatccaccttatcggcaatgaagtcggtcccgtagaccgtctggccgtccttctcgtacttggtattccgaatcttgccctgcacgaataccagcgaccccttgcccaaatacttgccgtgggcctcggcctgagagccaaaacacttgatgcggaagaagtcggtgcgctcctgcttgtcgccggcatcgttgcgccacatctaggtactaaaacaattcatccagtaaaatataatattttattttctcccaatcaggcttgatccccagtaagtcaaaaaatagctcgacatactgttcttccccgatatcctccctgatcgaccggacgcagaaggcaatgtcataccacttgtccgccctgccgcttctcccaagatcaataaagccacttactttgccatctttcacaaagatgttgctgtctcccaggtcgccgtgggaaaagacaagttcctcttcgggcttttccgtctttaaaaaatcatacagctcgcgcggatctttaaatggagtgtcttcttcccagttttcgcaatccacatcggccagatcgttattcagtaagtaatccaattcggctaagcggctgtctaagctattcgtatagggacaatccgatatgtcgatggagtgaaagagcctgatgcactccgcatacagctcgataatcttttcagggctttgttcatcttcatactcttccgagcaaaggacgccatcggcctcactcatgagcagattgctccagccatcatgccgttcaaagtgcaggacctttggaacaggcagctttccttccagccatagcatcatgtccttttcccgttccacatcataggtggtccctttataccggctgtccgtcatttttaaatataggttttcattttctcccaccagcttatataccttagcaggagacattccttccgtatcttttacgcagcggtatttttcgatcagttttttcaattccggtgatattctcattttagccatttattatttccttcctcttttctacagtatttaaagataccccaagaagctaattataacaagacgaactccaattcactgttccttgcattctaaaaccttaaataccagaaaacagctttttcaaagttgttttcaaagttggcgtataacatagtatcgacggagccgattttgaaaccacaattatgggtgatgctgccaactcgagagcgggccgggagggttcgagaagggggggcaccccccttcggcgtgcgcggtcacgcgcacagggcgcagccctggttaaaaacaaggtttataaatattggtttaaaagcaggttaaaagacaggttagcggtggccgaaaaacgggcggaaacccttgcaaatgctggattttctgcctgtggacagcccctcaaatgtcaataggtgcgcccctcatctgtcagcactctgcccctcaagtgtcaaggatcgcgcccctcatctgtcagtagtcgcgcccctcaagtgtcaataccgcagggcacttatccccaggcttgtccacatcatctgtgggaaactcgcgtaaaatcaggcgttttcgccgatttgcgaggctggccagctccacgtcgccggccgaaatcgagcctgcccctcatctgtcaacgccgcgccgggtgagtcggcccctcaagtgtcaacgtccgcccctcatctgtcagtgagggccaagttttccgcgaggtatccacaacgccggcggccggccgcggtgtctcgcacacggcttcgacggcgtttctggcgcgtttgcagggccatagacggccgccagcccagcggcgagggcaaccagcccggtgagctctagtggactgatgggctgcctgtatcgagtggtgattttgtgccgagctgccggtcggggagctgttggctggct

SEQ ID NO: 6 - DNA, 18808 bp, Artificial sequence
  Synthetic nucleotide
ggtggcaggatatattgtggtgtaaacaaattgacgcttagacaacttaataacacattgcggacgtttttaatgtactggggtggttttggtaccgggccccccctcgaggtcgacggtatcgataagcttgatatcgaattcctgcaggcctcctaggactagttcccaggtcaacatggtggagcacgacactctcgtctactccaagaatatcaaagatacagtctcagaagaccagagggctattgagacttttcaacaaagggtaatatcgggaaacctcctcggattccattgcccagctatctgtcacttcatcgaaaggacagtagaaaaggaagatggcttctacaaatgccatcattgcgataaaggaaaggctatcgttcaagatgcctctaccgacagtggtcccaaagatgagacttttcaacaaagggtaatatcgggaaacctcctcggattccattgcccagctatctgtcacttcatcgaaaggacagtagaaaaggaagatggcttctacaaatgccatcattgcgataaaggaaaggctatcgttcaagatgcctctaccgacagtggtcccaaagatggacccccacccacgaggaacatcgtggaaaaagaagacgttccaaccacgtcttcaaagcaagtggattgatgtgatatctccactgacgtaagggatgacgcacaatcccactatccttcgcaagacccttcctctatataaggaagttcatttcatttggagaggagagacccatatatttcataaagggtcatctggtctgtatcacatagctgattagtcctcatgatgctgggcaagaatgcttgtgggaagatccgcccgcaacctgctcaataacacatgatcaggaagtgacacaatctgcgactattaacaaaaacccaaccacctctaaccttcttcatctcatccttcataatggcaaacatcaacatcccggatgacctggtatccaagtactcggaagatgtaaaggatctcacacaaaaggcaggtagcattccaagttctaaatcattgatcccccaaaccgcattcacccctgatgaattgaggagatctttgaagttctggcaagtgaccaccaaagtggcagcaactgtagcatccgactggactaatttggcgactgcattaaccaacgggacattcagtgcgacgcatctcaggacactgtgtgagctggcattcaacctcaaggccccaactggaacaggcacaatcttcattcatgagattgctgacccgtggaaagggtgcattgatgctaatgctcccgacactgtagcgatacctgcgactgacagtgatgttaatatgtctactgtcagctcaggttctaccgcctctgggacggaagatgcagaggtcaaaatgaaggcgatctcattcctatgctgcacattgctcaggctgtctgtgaaagagccgtcacacatcatgcaagcgatcaacagcatacgacagaggttcggctccctgtacggtgtctcctcccctaccttgaatagtgttacattcaacaggagccaactttctcgcatcaagcaaggcattgaaacatatccctctgctcggggtacagtattctattacaccaggtatgccgatgtcacgcatgggtatactaccaaagagtatgggatatgcaggttcctactgttccaacaccttgaacttgaggggatgcacatctataagatgctcttagctctacttggcgagtggtcaacagttccaattgggctcttgctcacctggatcaggaacccgaggtctaagttggccataaatgagattgtcaagatagtcaaggagcttgataagccggaagtggacaagggctggaaatatgctcggatggtcaataacacattcttccttgatctatcctctagaaggaacacttacctatgtgctgtgttggcctccctgaacaagaagaacgttccgcaaggatcaggggaatatgctgaccccactaacatagctgtgatcaagaatatggaccaatctgtgaaatcacaggttgccacagatgtgaccctgattgagagaatttatgagaaatatctggtgtctgcaggtggagacgaggcaggaacagcatatgctctgtctcgcggagttaagagatcctcgcctcaaagtcaggaaggcggtcaatccggccagtctggaggaacacccatggaagtcgacggagcatctggtagaggagcagcaggtccggccccgaagaagacacgatcaggcttatagatctcagctcggtatcaatagcctacatgagaagatgtaccacgtgccatacgctcctgaacctatgtgtgatcccaatggaccaacaataatagcagagtctgtcataaccctgccgttttaatcaattatcatgtgcttcccatgttgttgtttcaatctattaagaaaaacccaacctcaggatggactacaaagaccatgacggtgattataaagatcatgatatcgattacaaggatgacgatgacaagatgtcaccaacttgcataccttccggtcagccctgcccatataacgagaattgttgttctcaaagctgtacatttaaggaaaatgaaaacggaaataccgtaaaaagatgcgattaaatctcagctcggtatcaatagcctacatgagaagatgtaccacgtgccatacgctcctgaacctatgtgtgatcccaatggaccaacaataatagcagagtctgtcataaccctgccgttttaatcaattatcatgtgcttcccatgttgttgtttcaatctattaagaaaaacccaacctcagcatggactacaaagaccatgacggtgattataaagatcatgatatcgattacaaggatgacgatgacaagatggccaggaacctactgacgaacggtgaaggactgtacgcaggccaatccctagttgtagaacagtacacttttataatgcaggatgactgcaaccttgtgctgtacgaatacagcacccccatctgggcctcaaacaccggcgtcaccggcaaaaacgggtgcagggctgtcatgcaggctgatggcaactttgtggtctacgatgttaacggacgtcccgtctgggccagtaacagcagaagagggaacgggaactatatcctggtgcttcagaaggacaggaacgttgttatttacggatctgatatttggttctactggacctactaaatctcagctcggtatcaatagcctacatgagaagatgtaccacgtgccatacgctcctgaacctatgtgtgatcccaatggaccaacaataatagcagagtctgtcataaccctgccgttttaatcaattatcatgtgcttcccatgttgttgtttcaatctattaagaaaaacccaacctcagcatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaaatctcagctcggtatcaatagcctacatgagaagatgtaccacgtgccatacgctcctgaacctatgtgtgatcccaatggaccaacaataatagcagagtctgtcataaccctgccgttttaatcaattatcatgtgcttcccatgttgttgtttcaatctattaagaaaaacccaacctcaggatgaatcgttactcccgtcgttctagacatcccaaccctcctgtccccaatcaggaagaacccgagcgcgacccgaatcacatagatcaagacctcgcagacttggctcaacccttggtcctaaaagaaagacacgcagtaatggcaccaactcagcccagccttagcgatgtgatcaatgaggaaagacaggctcccatcacgtttggtaatcctccagaggtcatggcgaacgctaggctatcagcgctgggttatgacaatctcacggagagggagaagaggatccttgctgtaggaatcagatgcggagaggctgccaaagactatcattcgctcactaccaccaaaaagtggattgaggatgagctgaagtctcaaatggtggctctcgcatcatcaacaaggactctcactgaggctgcatcacttcacacgacctttgcaatgctgcattctccatccattaaaagaaaggcagaagcaatgtctcacatatctcaaggcgaagagagcattgacatttctaagctgaataagactggaatggaggacatctgggtggcaatggagagtgagtcaaaagaggatgcagtagacacgtatctgagaaacattctggaagtggacccgacacaattctatgcaatagacggctggggcctctacctggacttcattcccacttggcattatatagctgcagggaagaattcggcgcaatttaagacgagttatgctgacgagattgtagagcagcgtgctgcattcgagagggtcctctctaagcgccctagggttgagatctaagcctgtatgtcactccactccccaataatgtccacgtcagcctcatgacaccctgctattaagaaaaacccaacaatggagccctctgcagagatccccctcgggaaaggcaaggaaaccgggagaagccctctgatcgcaacaaggaggaagaccgggctggtcaaagccaagataaagaccaattcatccttctctatgttcaggagagcagatactatgcgaacccatgcacagttcaccaacattactgtggggtggaagtcgctttgtcctatcactgcagaaggacaggtcaatgtagccatataccatgagagcacccgagtccctgttctcaatctatggagtcctgtgtcctctagttggaagcacctggcaacaggcagtctaggctttgttagtctgaaccactgtccatatgtggttgaaggaagaatatcgggattcgagggagaagaagcagggttggtgacacttacacttcatcttgacactggattggagagtgatgacatccagaagtgtcgtttattttctgaacatccggagttagcgggtggatcactatatttatacacttcctacacaagtagccccataccggacagcaatctccctgccttaaggactgagatcgatgacactatcgctgcaatttggaggatgagtggagggaagatgtctatcaccacaagccatgctctagggctgattgccaccttcagggttaaaatagagtctttacttggggaatacccggatgatgcattctctgctcaggttgagaatcttgtgaaggattttctccttgtttcatcgacagctgagataaccagatcttacaggacctgccttgcaaaagtgctgtcgtcgatgaccacgtatgacaagaagtactgggacaccatcataacaggtccctcctccatatgacactcgccatcgcagcagctggagctcatattcccatcatcaagcacatctgcaagaataaaaatccgctcccacccttcattcatcctcacaaccaccctccgcccttgttaagaaaaacccaacatggatgctcctatcaatccttcctctcccacccggtcttcctatgagacagccctagagaggcaatggttgatcaaggatgaaccgtcacaccatgttgacttgggatgtcacttcacattagagatcaaagcattctcacccaaaatgattgagttgctggagactggtagcctgactctaggatccgtgtatcaagggctgtacaatttgatcaaggacaaagactgcaaggggctggagctagatcgggacaccacccatgggaacaaattgcatgtcttggcggcagctttcctgtgtgtgctcaggaagtatgagaaccaggtgcggttttacaatgtcaaaaccaaggactctctgacgggcgaaatccagtatatgtctagggtacacatcggagatgataacttggggctcttcgatccagacacctacaggatgataggagggtcatttccggaagggatctaccgactgaacctccgcacacaactcacacccaggggagaagatgacggtctggaaatcataatgggtaccattctaaccgtccccgtgcgcggtcctgctgaccgacagcctctcggagtattgatggacagacaactaagacagttcccggtagggaccaacccttggtcgtccttgggggaggcgaaacctaaagggagatcgttgtcactcctgcgtggaatccttatgtaattctgcatatcacctggttgatatccaataagaatgccactgcccagcattaccccacgattaagaaaaacccaacttacacagacgcaatcatgtctctcattcacctcatctacttccccaccatcctgtctctcatcctaggtacagagggcacaccagcattgacttatggcaacctggctgacaatcagcatggaaatgaggagcaaggggatgttgatatgtatcctctatatgagtgcagtaagaagggtgcaggagtctctagcaataactggtacggagcctgccgaggagcttgctctatgaccaaaaatactactgatgtccacatggagatatattacaggaatgacagtgtcggttggatagatgtattgtctatccagactacagaaatctggaagcactcacatgtaacctggtacggagagtgtgaacacaatacaaaatatgggagctcgaacacagcacctgtctccgtcattctggagcggttggagttccttcacaatgggatgtcatcatggccatacggtcatagcattcatgttcatgacactatctcaagccctgactgtagctattttggtgatgtgtccagatccggatggagattgataatatctaaacgtgctcttgaactgaaatcagatattgcaggagagggctatattgtagaccccgatgtggggtatgtatatcctgttgcggacggaattgggaaaggtaggacctggcatgtatggaaagctagctctgttccgtcttctggctgctacttcaaatcggcaggaattactaattgtaccctactaatggatacattcctctattcatgtccaagcctcaatatagccttctcagctcgggtcgggaaacacataaagagcacgtgcgtcggagatatgaatatatcaacagacggtgtgacttatcagactctcgggagcacggatcaagggtcgatatccacacagttggtatcattatggcatcaatcacaggaagctctcatacagcaacttattttgactatcaacgaggccctaggaaaaattgagacaagctactgtgagtccacttgtgatttgaccgagatgttaatctcccagcataccgagcacccgatggtaattgagacgcctgtcgggccatggctaccatacagcatagacggtaaagtttctgttctcccttgtcaaggtggtcaggatctggtggtgatcaagcctatagagatctgcgcttctccattcatgctcaaagttaaatcactcaagtctctagacacctactggtgggtccctacagatagccacgtggctccgttcacgcaatgcagcaacagggacgatgaagaaatgatcatcaagatacaacggaagaagccattgatatttgaattctggagaggagcctataagcttgattacccgtataactctagtggaatctggctggataaccctggaggtcatattcatagaagctctaaatggttccctacgttggatagtctatcctacagttctcctataaacttgccactgatcagcaaaggtataaggaaacatattcaagccatcgtcagtaccaccgagatcacatctggaggtaacgttacatcatggacccactcgatttcgactattacagaggccatgttcgactcagctgcgatagccgcgggtcgggtcgtgatgtggtggtacacactagaagaaagcgtcaaaaggtatctgactgtagcgttcggtctagtactaactgtcatcaccatttgggctatgtcaaaattattcttccgatcgtcgccaacatatgccagggcgcccacaaatatagaatgggtaaggtcatagacatcgcagtgtccacccacctcatctccatggagcccgatccacaataaaatgccatctatgtatcaaattaatcctatatgtatattaagaaaaacccaattccaatggatccagattatcctgatcttgatcccgagtctcttgatgttctcaacagcctacaagaagggtacgatgaggaggaagaagaggacagcggagtgtcactcaatggaatgggggactaccaccttaagtcagcactcaggtcgtacacagatatgatgaagcacccaatcttcaagaaggagttctccaaggcagtgattaactttggtatatctcacgactcgatgatgtcccagatagaaacaatgttctacatcctgaagagtactccattgtcaatgcaccttgggaccctttatggtgatctcttcactcgtcagagcacgctcggccacagcgaagatgtatttgacattatccaagcagagatacgaactcttcatcctcacctcctcctagagatgacacctgccaaggtcgaggagatgattctgattgctgacaggaagacatatgatgagagggcagcatctgcgttctggtccactctcataacaataaagaattacattccagcttggacagagaagggggcttgtactctggattggcctagtgtgtctctagacaagaagagtgggtacgtgaaagtgatggcaggtaaagatactaccatctacattgggactgatatttgtgtgatagagaagtatcctacagtaaaatgggctccactttcataccttttaaatggagctgacaaggtggcagaaaggctgaatgtgcgttactatagttgtctttgtgatcagcttgatataccagataggataacacttgatatgcttgatgaaatcatcaaagtgggggatgattgtcttcgagagatgggcaatgacggatacaatgtaattggatcctatgaggctttgcttgcaggaataatccaaaggagggacaaccagggactaataccggacagagatttgttgtggagaactactatggaagaatttacatccagccccggcaggaagtatctagaagagtggagcacaatgtttagttcattaagccctgaacagatagcctgtgctcatggactgtataggatctgggggcacccaatagttgatattcttggagggataaagaagatgcaagaggtggcatctgtgaagaagaatcctagacagagtgtactggatgaaataaggaggcaattcaaagaaatgttttttacctcatatcataggatacacaagcactaccctctacatacaatcttgaacagcctggataattcctatatactcaactgtctaaaggataacttgacgatcaacacatcagcaatctcctataacttccaggattgggactgtgtgatggtggagaaaaacttcgaagtgcctttctcttggaacctggttcacaatttgaaagataaagctatttccccgaacaggcaggagatttaccagacgatatcaactcgtggtacaatattcgggtctcagaaccgcagagggatactgaaatcactcacgatggagactgtccaactccgagactttcttcaaggcataaatgacaacgggttggatgacatagacaaaattattggggtctatcctaaagagcgtgagctgaagataaaggctagactgttctccctcatgtcattccggctcagattgtactgtgtgtcaacagaggcattattaggggacaaaatactcaagtatttcccgcaaatcacaatgtctcttgatatgctcacgatgataaagaagatgttcaaagtgtcctcccaaactactagagaggatgattcagtcacagtcatattcaatcttgacttcataaagtggaatctccagatgagacgtaatatatgtgagcctgtcttctcacaattaggtaaactctttggcatggagaatctgttcaataggactcatgagaccttccgagatagcctgatatacctctgttcgggggaaggagtactgtcagccgatccagtctacggagtattcccggacggtacatgggcatgggcgggtgatgagtcggggaaggaagggcttcgtcagaagggttggacaattctgactgtcgttacaatcatgttgattgcaaagcgacacaatgttgatgtatcactaatgggaggaggagacaatcaagtccttggtatcaccatatcaggagtgaccagagatcttcaaggagagctgattacagaatcggcctcactcgcccgaaggacaatcaagcaatttaccgaggacttattaaccactttctccgatctggggctccctttaaaagcatctgaaacatgggtcagtgactctctcttcatgtacaacaaacacatgttttacaagggaattcctctcagatcacctctgaaagccatctcaaggatcttcccgctggcaaacgacacaattatgaccatagacaacatgatcaataatatatcatcaggggtcaaagcagcctgtatgaaggagagacacggaattccacttgtattcatgaaaacactagcatataggaaggttgccgagggagcactgatcctacatccattgacgacttgcttcaggacaccaacattgcctgaaaatggaactgtgacgcgtgacggacggaagcgatcagtgaaggtgactaggaatcagctgagatcattctttacgttatgtatggtgggatcttccactatgggactacctggtactatacatctccctgatatgatcatgaggggattccctgatccattgaccagctacctgtcattcatttcagaattggttcccaagatcatggacccaagactagcgagcatcatcgcaacatcgggtcatatgagcatcaatagaatgacagagtatgcgaaacttgtggaggatcctgtatctataaatcacgacgcccccacacatggactgaatgagatccgacagttatctcgagacttcttgatgaacacgaccctggcagccaacacccatcttaagtcccttttcaccctcctagacagacagagagaaaaagaattctacgacgcactatgttctgctgaggaactggatgtgaaggtgctgcacgaagtagccggagcatcactattcggatatacaaacggcattgcctctcgtattgaccagacaagaacggtgagagcgttaaacgagacagtcaacgttatgaaaaggctagctgatgcagaggcaagatatattgcatatctgttcgctagggacatacataagcatgacctcatcccggatcagtgttccagagtgactgcggacaggtttgtttctgcttctacctttgatatatatataataattatcattaattagtagtaatataatatttcaaatatttttttcaaaataaaagaatgtagtatatagcaattgcttttctgtagtttataagtgtgtatattttaatttataacttttctaatatatgaccaaaacatggtgatgtttaggtatcgatattattcatggaagaagaaggttttaggggtaacagtacctcacccgattgagatgtgtcaagtcacatcggcgacccaaactgcattcgaggatgctgttatatgttggagtgataatctgagcggatcgagaatctacaccacaatgggtctagggaagatatatcaaggttcctacaccaaggaaaggttcaaggccacagacattgctgccgcgtatgggaacgaggacatcctgacaaaagcagtcaggctgcagaagctaattaactggagatatgatgaagggagcagctttgctaggataattcaacttgctctcagagccatcactgatgcaagcactgacggattccatagatctaaggaggagatcaaaggggaattcgatcatagaaggggcatatcaggagatatcagcggaggaattccaaatttccttgtcacacccacctcgcatttctcgtgtaccacatcctcttggatctcacatagcaggggagggaaaaacgagaatatccatttccagagtgtactgatcaacttattatacagagccatggtctatcgaggctcattgcccggtctaccagagatggtctggtattcaaaagagagatgcagccattgcattattgatattgtggaaccgtctccagaacgaacaaccccagctttgtcagagatgccatcagcacctaacaatccttttgcattcatagagtcagtaaatgtgaagttggattatcatcaccaggttgagatagaaaaaggaatggaagagaccaacctactaaattctgagtggggaggctcatctttaacaagtgtggatgaggctggactggtcttgtttctgatgctgattggatcccgtcaagtgtcagaatcgtttttgttattgatgagggagaagatagaacctcgaccgctactgcaagcagcattgaacagagtcatccttgtcaggaaactcggattggataggttgttccctataagaaacaccaaatgcatcaatcttatactggggtctgacagaggaatcactgcatgccatgacggctttggcctggaactgcacgggggatcctgggagaaagggatagagtgtgacacctcctttctttacaatgatactcacctcaagccctgtaaattgcatgttaaccttcatcttcaaaatgtgcctctccagctggccttggcgcatgcttcctataatcctgatgtttgtgagtgtcttgagtgcagagccatcattgaagatagagtctctaagaggatggtagggcaatataagagctggagatgccagtatcatgcggtcctgtccttctctccaatcatcatgagggtccacagtgagaggataatcaaaaatgagggagctatatatgaaggggatctgcatgtcccttatgtcgaggagatcattgctctggagaactcacaaaaattggcaatagatgtgacgtcatggagactgccgctgctgatgcacgcaacatgggagtctatcctgcctcagctgtatattactatgaaggcctgcctggtttgtcttcccttggagggtgtgattgtggatgatgacctggcactgatcaatctggtgtccaaggtgttggtcgatctgaggaggaccatccaggtctatatcagagcagggacgttctccgggtcggagatcaacaataagtatgacaacatcaggctgcttccagaggatcttagggacagcattcacatcctaacaaccacagacaaaccagatcctgctgcaactgtccaactggcagcggatgacacatcgcctcttgactgtgctgtgtaccatctcatctggggcgatacctggggcatgacgaggaaggtgcacggaagaattctcattccatcctctcgtgtgacaactggattcgcaggggtcatggtgacagatcactacgcaactgtggcagtattggagcttgctggtgctgttaacctgtgggagaaaaagctcattgatcttgaacatagaaacaacctcgatgttccgttgagcatgaatcgtgtgttggcaggttctagactcgggacccgggggataagatgggctcaatggacgtcgcctgcaggcctagagatcgttgtcaggaaagtgagggcaaagctgtcatcaatctcgggaaaccctggaaatgccgccaaatggaggaggaactgccagaaggtcctcaaggccttgatgatctccctgtatgctcattcgggcgaggacatgatgaggaatgcaagcggccttgtcaaggtgaacatacatgggaccctttcctctgtcaccgctgtgtgcgacccgtcggcggagagcagagtgcagagggcacagttcctgttcctaaaagaaaaggctcccaatgggcctttcattctaaggagcaggttggagaggcgcataaatctcctctcagttgtgtccgacctgctcgggtgatcaagaagtattaagaaaaaccctcatgttcacgtggttagaggggttacacccatactacatatcaaggtctttgacaatagtgtatttcgtgagaatgccatgtttttctgggtctctgggtcggcatggcatctccacctcctcgcggtccgacctgggcatccgaaggaggacgtccactcggatggctaagggagagctcgaattcggtacgctgaaatcaccagtctctctctacaaatctatctctctctattttctccataaataatgtgtgagtagtttcccgataagggaaattagggttcttatagggtttcgctcatgtgttgagcatataagaaacccttagtatgtatttgtatttgtaaaatacttctatcaataaaatttctaattcctaaaaccaaaatccagtactaaaatccagatccccaattcactggccgtcgttttacaacgtcgtgactgggaaaaccctggcgttacccaacttaatcgcctgtttaaactatcagtgtttgacagggacctgcagcccggccgggggatccactagcagattgtcgtttcccgccttcagtttaaactatcagtgtttgacaggatatattggcgggtaaacctaagagaaaagagcgtttattagaataatcggatatttaaaagggcgtgaaaaggtttatccgttcgtccatttgtatgtgcatgccaaccacaggagatctcagtaaagcgctggctgaacccccagccggaactgaccccacaaggccctagcgtttgcaatgcaccaggtcatcattgacccaggcgtgttccaccaggccgctgcctcgcaactcttcgcaggcttcgccgacctgctcgcgccacttcttcacgcgggtggaatccgatccgcacatgaggcggaaggtttccagcttgagcgggtacggctcccggtgcgagctgaaatagtcgaacatccgtcgggccgtcggcgacagcttgcggtacttctcccatatgaatttcgtgtagtggtcgccagcaaacagcacgacgatttcctcgtcgatcaggacctggcaacgggacgttttcttgccacggtccaggacgcggaagcggtgcagcagcgacaccgattccaggtgcccaacgcggtcggacgtgaagcccatcgccgtcgcctgtaggcgcgacaggcattcctcggccttcgtgtaataccggccattgatcgaccagcccaggtcctggcaaagctcgtagaacgtgaaggtgatcggctcgccgataggggtgcgcttcgcgtactccaacacctgctgccacaccagttcgtcatcgtcggcccgcagctcgacgccggtgtaggtgatcttcacgtccttgttgacgtggaaaatgaccttgttttgcagcgcctcgcgcgggattttcttgttgcgcgtggtgaacagggcagagcgggccgtgtcgtttggcatcgctcgcatcgtgtccggccacggcgcaatatcgaacaaggaaagctgcatttccttgatctgctgcttcgtgtgtttcagcaacgcggcctgcttggcctcgctgacctgttttgccaggtcctcgccggcggtttttcgcttcttggtcgtcatagttcctcgcgtgtcgatggtcatcgacttcgccaaacctgccgcctcctgttcgagacgacgcgaacgctccacggcggccgatggcgcgggcagggcagggggagccagttgcacgctgtcgcgctcgatcttggccgtagcttgctggaccatcgagccgacggactggaaggtttcgcggggcgcacgcatgacggtgcggcttgcgatggtttcggcatcctcggcggaaaaccccgcgtcgatcagttcttgcctgtatgccttccggtcaaacgtccgattcattcaccctccttgcgggattgccccgactcacgccggggcaatgtgcccttattcctgatttgacccgcctggtgccttggtgtccagataatccaccttatcggcaatgaagtcggtcccgtagaccgtctggccgtccttctcgtacttggtattccgaatcttgccctgcacgaataccagcgaccccttgcccaaatacttgccgtgggcctcggcctgagagccaaaacacttgatgcggaagaagtcggtgcgctcctgcttgtcgccggcatcgttgcgccacatctaggtactaaaacaattcatccagtaaaatataatattttattttctcccaatcaggcttgatccccagtaagtcaaaaaatagctcgacatactgttcttccccgatatcctccctgatcgaccggacgcagaaggcaatgtcataccacttgtccgccctgccgcttctcccaagatcaataaagccacttactttgccatctttcacaaagatgttgctgtctcccaggtcgccgtgggaaaagacaagttcctcttcgggcttttccgtctttaaaaaatcatacagctcgcgcggatctttaaatggagtgtcttcttcccagttttcgcaatccacatcggccagatcgttattcagtaagtaatccaattcggctaagcggctgtctaagctattcgtatagggacaatccgatatgtcgatggagtgaaagagcctgatgcactccgcatacagctcgataatcttttcagggctttgttcatcttcatactcttccgagcaaaggacgccatcggcctcactcatgagcagattgctccagccatcatgccgttcaaagtgcaggacctttggaacaggcagctttccttccagccatagcatcatgtccttttcccgttccacatcataggtggtccctttataccggctgtccgtcatttttaaatataggttttcattttctcccaccagcttatataccttagcaggagacattccttccgtatcttttacgcagcggtatttttcgatcagttttttcaattccggtgatattctcattttagccatttattatttccttcctcttttctacagtatttaaagataccccaagaagctaattataacaagacgaactccaattcactgttccttgcattctaaaaccttaaataccagaaaacagctttttcaaagttgttttcaaagttggcgtataacatagtatcgacggagccgattttgaaaccacaattatgggtgatgctgccaactcgagagcgggccgggagggttcgagaagggggggcaccccccttcggcgtgcgcggtcacgcgcacagggcgcagccctggttaaaaacaaggtttataaatattggtttaaaagcaggttaaaagacaggttagcggtggccgaaaaacgggcggaaacccttgcaaatgctggattttctgcctgtggacagcccctcaaatgtcaataggtgcgcccctcatctgtcagcactctgcccctcaagtgtcaaggatcgcgcccctcatctgtcagtagtcgcgcccctcaagtgtcaataccgcagggcacttatccccaggcttgtccacatcatctgtgggaaactcgcgtaaaatcaggcgttttcgccgatttgcgaggctggccagctccacgtcgccggccgaaatcgagcctgcccctcatctgtcaacgccgcgccgggtgagtcggcccctcaagtgtcaacgtccgcccctcatctgtcagtgagggccaagttttccgcgaggtatccacaacgccggcggccggccgcggtgtctcgcacacggcttcgacggcgtttctggcgcgtttgcagggccatagacggccgccagcccagcggcgagggcaaccagcccggtgagctctagtggactgatgggctgcctgtatcgagtggtgattttgtgccgagctgccggtcggggagctgttggctggct

SEQ ID NO: 7 - DNA, 18285 bp, Artificial sequence
  Synthetic nucleotide
ggtggcaggatatattgtggtgtaaacaaattgacgcttagacaacttaataacacattgcggacgtttttaatgtactggggtggttttggtaccgggccccccctcgaggtcgacggtatcgataagcttgatatcgaattcctgcaggcctcctaggactagttccccaggtcaacatggtggagcacgacactctcgtctactccaagaatatcaaagatacagtctcagaagaccagagggctattgagacttttcaacaaagggtaatatcgggaaacctcctcggattccattgcccagctatctgtcacttcatcgaaaggacagtagaaaaggaagatggcttctacaaatgccatcattgcgataaaggaaaggctatcgttcaagatgcctctaccgacagtggtcccaaagatggacccccacccacgaggaacatcgtggaaaaagaagacgttccaaccacgtcttcaaagcaagtggattgatgtgatatctccactgacgtaagggatgacgcacaatcccactatccttcgcaagacccttcctctatataaggaagttcatttcatttggagaggagagacccatatatttcataaagggtcatctggtctgtatcacatagctgattagtcctcatgatgctgggcaagaatgcttgtgggaagatccgcccgcaacctgctcaataacacatgatcaggaagtgacacaatctgcgactattaacaaaaacccaaccacctctaaccttcttcatctcatccttcataatggcaaacatcaacatcccggatgacctggtatccaagtactcggaagatgtaaaggatctcacacaaaaggcaggtagcattccaagttctaaatcattgatcccccaaaccgcattcacccctgatgaattgaggagatctttgaagttctggcaagtgaccaccaaagtggcagcaactgtagcatccgactggactaatttggcgactgcattaaccaacgggacattcagtgcgacgcatctcaggacactgtgtgagctggcattcaacctcaaggccccaactggaacaggcacaatcttcattcatgagattgctgacccgtggaaagggtgcattgatgctaatgctcccgacactgtagcgatacctgcgactgacagtgatgttaatatgtctactgtcagctcaggttctaccgcctctgggacggaagatgcagaggtcaaaatgaaggcgatctcattcctatgctgcacattgctcaggctgtctgtgaaagagccgtcacacatcatgcaagcgatcaacagcatacgacagaggttcggctccctgtacggtgtctcctcccctaccttgaatagtgttacattcaacaggagccaactttctcgcatcaagcaaggcattgaaacatatccctctgctcggggtacagtattctattacaccaggtatgccgatgtcacgcatgggtatactaccaaagagtatgggatatgcaggttcctactgttccaacaccttgaacttgaggggatgcacatctataagatgctcttagctctacttggcgagtggtcaacagttccaattgggctcttgctcacctggatcaggaacccgaggtctaagttggccataaatgagattgtcaagatagtcaaggagcttgataagccggaagtggacaagggctggaaatatgctcggatggtcaataacacattcttccttgatctatcctctagaaggaacacttacctatgtgctgtgttggcctccctgaacaagaagaacgttccgcaaggatcaggggaatatgctgaccccactaacatagctgtgatcaagaatatggaccaatctgtgaaatcacaggttgccacagatgtgaccctgattgagagaatttatgagaaatatctggtgtctgcaggtggagacgaggcaggaacagcatatgctctgtctcgcggagttaagagatcctcgcctcaaagtcaggaaggcggtcaatccggccagtctggaggaacacccatggaagtcgacggagcatctggtagaggagcagcaggtccggccccgaagaagacacgatcaggcttatagatctcagctcggtatcaatagcctacatgagaagatgtaccacgtgccatacgctcctgaacctatgtgtgatcccaatggaccaacaataatagcagagtctgtcataaccctgccgttttaatcaattatcatgtgcttcccatgttgttgtttcaatctattaagaaaaacccaacctcaggttcagagcaccagtggtctagtggtagaatagtaccctgccacggtacagacccgggttcgattcccggctggtgcatgtcgtcccagcctgaaaaagttttagagctagaaatagcaagttaaaataaggctagtccgttatcaacttgaaaaagtggcaccgagtcggtgcttcagagcaccagtggtctagtggtagaatagtaccctgccacggtacagacccgggttcgattcccggctggtgcaggagaaaataaaaaaagcatgttttagagctagaaatagcaagttaaaataaggctagtccgttatcaacttgaaaaagtggcaccgagtcggtgcttcagagcaccagtggtctagtggtagaatagtaccctgccacggtacagacccgggttcgattcccggctggtgcaatctcagctcggtatcaatagcctacatgagaagatgtaccacgtgccatacgctcctgaacctatgtgtgatcccaatggaccaacaataatagcagagtctgtcataaccctgccgttttaatcaattatcatgtgcttcccatgttgttgtttcaatctattaagaaaaacccaacctcgggatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaaatctcagctcggtatcaatagcctacatgagaagatgtaccacgtgccatacgctcctgaacctatgtgtgatcccaatggaccaacaataatagcagagtctgtcataaccctgccgttttaatcaattatcatgtgcttcccatgttgttgtttcaatctattaagaaaaacccaacctcaggatgaatcgttactcccgtcgttctagacatcccaaccctcctgtccccaatcaggaagaacccgagcgcgacccgaatcacatagatcaagacctcgcagacttggctcaacccttggtcctaaaagaaagacacgcagtaatggcaccaactcagcccagccttagcgatgtgatcaatgaggaaagacaggctcccatcacgtttggtaatcctccagaggtcatggcgaacgctaggctatcagcgctgggttatgacaatctcacggagagggagaagaggatccttgctgtaggaatcagatgcggagaggctgccaaagactatcattcgctcactaccaccaaaaagtggattgaggatgagctgaagtctcaaatggtggctctcgcatcatcaacaaggactctcactgaggctgcatcacttcacacgacctttgcaatgctgcattctccatccattaaaagaaaggcagaagcaatgtctcacatatctcaaggcgaagagagcattgacatttctaagctgaataagactggaatggaggacatctgggtggcaatggagagtgagtcaaaagaggatgcagtagacacgtatctgagaaacattctggaagtggacccgacacaattctatgcaatagacggctggggcctctacctggacttcattcccacttggcattatatagctgcagggaagaattcggcgcaatttaagacgagttatgctgacgagattgtagagcagcgtgctgcattcgagagggtcctctctaagcgccctagggttgagatctaagcctgtatgtcactccactccccaataatgtccacgtcagcctcatgacaccctgctattaagaaaaacccaacaatggagccctctgcagagatccccctcgggaaaggcaaggaaaccgggagaagccctctgatcgcaacaaggaggaagaccgggctggtcaaagccaagataaagaccaattcatccttctctatgttcaggagagcagatactatgcgaacccatgcacagttcaccaacattactgtggggtggaagtcgctttgtcctatcactgcagaaggacaggtcaatgtagccatataccatgagagcacccgagtccctgttctcaatctatggagtcctgtgtcctctagttggaagcacctggcaacaggcagtctaggctttgttagtctgaaccactgtccatatgtggttgaaggaagaatatcgggattcgagggagaagaagcagggttggtgacacttacacttcatcttgacactggattggagagtgatgacatccagaagtgtcgtttattttctgaacatccggagttagcgggtggatcactatatttatacacttcctacacaagtagccccataccggacagcaatctccctgccttaaggactgagatcgatgacactatcgctgcaatttggaggatgagtggagggaagatgtctatcaccacaagccatgctctagggctgattgccaccttcagggttaaaatagagtctttacttggggaatacccggatgatgcattctctgctcaggttgagaatcttgtgaaggattttctccttgtttcatcgacagctgagataaccagatcttacaggacctgccttgcaaaagtgctgtcgtcgatgaccacgtatgacaagaagtactgggacaccatcataacaggtccctcctccatatgacactcgccatcgcagcagctggagctcatattcccatcatcaagcacatctgcaagaataaaaatccgctcccacccttcattcatcctcacaaccaccctccgcccttgttaagaaaaacccaacatggatgctcctatcaatccttcctctcccacccggtcttcctatgagacagccctagagaggcaatggttgatcaaggatgaaccgtcacaccatgttgacttgggatgtcacttcacattagagatcaaagcattctcacccaaaatgattgagttgctggagactggtagcctgactctaggatccgtgtatcaagggctgtacaatttgatcaaggacaaagactgcaaggggctggagctagatcgggacaccacccatgggaacaaattgcatgtcttggcggcagctttcctgtgtgtgctcaggaagtatgagaaccaggtgcggttttacaatgtcaaaaccaaggactctctgacgggcgaaatccagtatatgtctagggtacacatcggagatgataacttggggctcttcgatccagacacctacaggatgataggagggtcatttccggaagggatctaccgactgaacctccgcacacaactcacacccaggggagaagatgacggtctggaaatcataatgggtaccattctaaccgtccccgtgcgcggtcctgctgaccgacagcctctcggagtattgatggacagacaactaagacagttcccggtagggaccaacccttggtcgtccttgggggaggcgaaacctaaagggagatcgttgtcactcctgcgtggaatccttatgtaattctgcatatcacctggttgatatccaataagaatgccactgcccagcattaccccacgattaagaaaaacccaacttacacagacgcaatcatgtctctcattcacctcatctacttccccaccatcctgtctctcatcctaggtacagagggcacaccagcattgacttatggcaacctggctgacaatcagcatggaaatgaggagcaaggggatgttgatatgtatcctctatatgagtgcagtaagaagggtgcaggagtctctagcaataactggtacggagcctgccgaggagcttgctctatgaccaaaaatactactgatgtccacatggagatatattacaggaatgacagtgtcggttggatagatgtattgtctatccagactacagaaatctggaagcactcacatgtaacctggtacggagagtgtgaacacaatacaaaatatgggagctcgaacacagcacctgtctccgtcattctggagcggttggagttccttcacaatgggatgtcatcatggccatacggtcatagcattcatgttcatgacactatctcaagccctgactgtagctattttggtgatgtgtccagatccggatggagattgataatatctaaacgtgctcttgaactgaaatcagatattgcaggagagggctatattgtagaccccgatgtggggtatgtatatcctgttgcggacggaattgggaaaggtaggacctggcatgtatggaaagctagctctgttccgtcttctggctgctacttcaaatcggcaggaattactaattgtaccctactaatggatacattcctctattcatgtccaagcctcaatatagccttctcagctcgggtcgggaaacacataaagagcacgtgcgtcggagatatgaatatatcaacagacggtgtgacttatcagactctcgggagcacggatcaagggtcgatatccacacagttggtatcattatggcatcaatcacaggaagctctcatacagcaacttattttgactatcaacgaggccctaggaaaaattgagacaagctactgtgagtccacttgtgatttgaccgagatgttaatctcccagcataccgagcacccgatggtaattgagacgcctgtcgggccatggctaccatacagcatagacggtaaagtttctgttctcccttgtcaaggtggtcaggatctggtggtgatcaagcctatagagatctgcgcttctccattcatgctcaaagttaaatcactcaagtctctagacacctactggtgggtccctacagatagccacgtggctccgttcacgcaatgcagcaacagggacgatgaagaaatgatcatcaagatacaacggaagaagccattgatatttgaattctggagaggagcctataagcttgattacccgtataactctagtggaatctggctggataaccctggaggtcatattcatagaagctctaaatggttccctacgttggatagtctatcctacagttctcctataaacttgccactgatcagcaaaggtataaggaaacatattcaagccatcgtcagtaccaccgagatcacatctggaggtaacgttacatcatggacccactcgatttcgactattacagaggccatgttcgactcagctgcgatagccgcgggtcgggtcgtgatgtggtggtacacactagaagaaagcgtcaaaaggtatctgactgtagcgttcggtctagtactaactgtcatcaccatttgggctatgtcaaaattattcttccgatcgtcgccaacatatgccagggcgcccacaaatatagaatgggtaaggtcatagacatcgcagtgtccacccacctcatctccatggagcccgatccacaataaaatgccatctatgtatcaaattaatcctatatgtatattaagaaaaacccaattccaatggatccagattatcctgatcttgatcccgagtctcttgatgttctcaacagcctacaagaagggtacgatgaggaggaagaagaggacagcggagtgtcactcaatggaatgggggactaccaccttaagtcagcactcaggtcgtacacagatatgatgaagcacccaatcttcaagaaggagttctccaaggcagtgattaactttggtatatctcacgactcgatgatgtcccagatagaaacaatgttctacatcctgaagagtactccattgtcaatgcaccttgggaccctttatggtgatctcttcactcgtcagagcacgctcggccacagcgaagatgtatttgacattatccaagcagagatacgaactcttcatcctcacctcctcctagagatgacacctgccaaggtcgaggagatgattctgattgctgacaggaagacatatgatgagagggcagcatctgcgttctggtccactctcataacaataaagaattacattccagcttggacagagaagggggcttgtactctggattggcctagtgtgtctctagacaagaagagtgggtacgtgaaagtgatggcaggtaaagatactaccatctacattgggactgatatttgtgtgatagagaagtatcctacagtaaaatgggctccactttcataccttttaaatggagctgacaaggtggcagaaaggctgaatgtgcgttactatagttgtctttgtgatcagcttgatataccagataggataacacttgatatgcttgatgaaatcatcaaagtgggggatgattgtcttcgagagatgggcaatgacggatacaatgtaattggatcctatgaggctttgcttgcaggaataatccaaaggagggacaaccagggactaataccggacagagatttgttgtggagaactactatggaagaatttacatccagccccggcaggaagtatctagaagagtggagcacaatgtttagttcattaagccctgaacagatagcctgtgctcatggactgtataggatctgggggcacccaatagttgatattcttggagggataaagaagatgcaagaggtggcatctgtgaagaagaatcctagacagagtgtactggatgaaataaggaggcaattcaaagaaatgttttttacctcatatcataggatacacaagcactaccctctacatacaatcttgaacagcctggataattcctatatactcaactgtctaaaggataacttgacgatcaacacatcagcaatctcctataacttccaggattgggactgtgtgatggtggagaaaaacttcgaagtgcctttctcttggaacctggttcacaatttgaaagataaagctatttccccgaacaggcaggagatttaccagacgatatcaactcgtggtacaatattcgggtctcagaaccgcagagggatactgaaatcactcacgatggagactgtccaactccgagactttcttcaaggcataaatgacaacgggttggatgacatagacaaaattattggggtctatcctaaagagcgtgagctgaagataaaggctagactgttctccctcatgtcattccggctcagattgtactgtgtgtcaacagaggcattattaggggacaaaatactcaagtatttcccgcaaatcacaatgtctcttgatatgctcacgatgataaagaagatgttcaaagtgtcctcccaaactactagagaggatgattcagtcacagtcatattcaatcttgacttcataaagtggaatctccagatgagacgtaatatatgtgagcctgtcttctcacaattaggtaaactctttggcatggagaatctgttcaataggactcatgagaccttccgagatagcctgatatacctctgttcgggggaaggagtactgtcagccgatccagtctacggagtattcccggacggtacatgggcatgggcgggtgatgagtcggggaaggaagggcttcgtcagaagggttggacaattctgactgtcgttacaatcatgttgattgcaaagcgacacaatgttgatgtatcactaatgggaggaggagacaatcaagtccttggtatcaccatatcaggagtgaccagagatcttcaaggagagctgattacagaatcggcctcactcgcccgaaggacaatcaagcaatttaccgaggacttattaaccactttctccgatctggggctccctttaaaagcatctgaaacatgggtcagtgactctctcttcatgtacaacaaacacatgttttacaagggaattcctctcagatcacctctgaaagccatctcaaggatcttcccgctggcaaacgacacaattatgaccatagacaacatgatcaataatatatcatcaggggtcaaagcagcctgtatgaaggagagacacggaattccacttgtattcatgaaaacactagcatataggaaggttgccgagggagcactgatcctacatccattgacgacttgcttcaggacaccaacattgcctgaaaatggaactgtgacgcgtgacggacggaagcgatcagtgaaggtgactaggaatcagctgagatcattctttacgttatgtatggtgggatcttccactatgggactacctggtactatacatctccctgatatgatcatgaggggattccctgatccattgaccagctacctgtcattcatttcagaattggttcccaagatcatggacccaagactagcgagcatcatcgcaacatcgggtcatatgagcatcaatagaatgacagagtatgcgaaacttgtggaggatcctgtatctataaatcacgacgcccccacacatggactgaatgagatccgacagttatctcgagacttcttgatgaacacgaccctggcagccaacacccatcttaagtcccttttcaccctcctagacagacagagagaaaaagaattctacgacgcactatgttctgctgaggaactggatgtgaaggtgctgcacgaagtagccggagcatcactattcggatatacaaacggcattgcctctcgtattgaccagacaagaacggtgagagcgttaaacgagacagtcaacgttatgaaaaggctagctgatgcagaggcaagatatattgcatatctgttcgctagggacatacataagcatgacctcatcccggatcagtgttccagagtgactgcggacaggtttgtttctgcttctacctttgatatatatataataattatcattaattagtagtaatataatatttcaaatatttttttcaaaataaaagaatgtagtatatagcaattgcttttctgtagtttataagtgtgtatattttaatttataacttttctaatatatgaccaaaacatggtgatgtttaggtatcgatattattcatggaagaagaaggttttaggggtaacagtacctcacccgattgagatgtgtcaagtcacatcggcgacccaaactgcattcgaggatgctgttatatgttggagtgataatctgagcggatcgagaatctacaccacaatgggtctagggaagatatatcaaggttcctacaccaaggaaaggttcaaggccacagacattgctgccgcgtatgggaacgaggacatcctgacaaaagcagtcaggctgcagaagctaattaactggagatatgatgaagggagcagctttgctaggataattcaacttgctctcagagccatcactgatgcaagcactgacggattccatagatctaaggaggagatcaaaggggaattcgatcatagaaggggcatatcaggagatatcagcggaggaattccaaatttccttgtcacacccacctcgcatttctcgtgtaccacatcctcttggatctcacatagcaggggagggaaaaacgagaatatccatttccagagtgtactgatcaacttattatacagagccatggtctatcgaggctcattgcccggtctaccagagatggtctggtattcaaaagagagatgcagccattgcattattgatattgtggaaccgtctccagaacgaacaaccccagctttgtcagagatgccatcagcacctaacaatccttttgcattcatagagtcagtaaatgtgaagttggattatcatcaccaggttgagatagaaaaaggaatggaagagaccaacctactaaattctgagtggggaggctcatctttaacaagtgtggatgaggctggactggtcttgtttctgatgctgattggatcccgtcaagtgtcagaatcgtttttgttattgatgagggagaagatagaacctcgaccgctactgcaagcagcattgaacagagtcatccttgtcaggaaactcggattggataggttgttccctataagaaacaccaaatgcatcaatcttatactggggtctgacagaggaatcactgcatgccatgacggctttggcctggaactgcacgggggatcctgggagaaagggatagagtgtgacacctcctttctttacaatgatactcacctcaagccctgtaaattgcatgttaaccttcatcttcaaaatgtgcctctccagctggccttggcgcatgcttcctataatcctgatgtttgtgagtgtcttgagtgcagagccatcattgaagatagagtctctaagaggatggtagggcaatataagagctggagatgccagtatcatgcggtcctgtccttctctccaatcatcatgagggtccacagtgagaggataatcaaaaatgagggagctatatatgaaggggatctgcatgtcccttatgtcgaggagatcattgctctggagaactcacaaaaattggcaatagatgtgacgtcatggagactgccgctgctgatgcacgcaacatgggagtctatcctgcctcagctgtatattactatgaaggcctgcctggtttgtcttcccttggagggtgtgattgtggatgatgacctggcactgatcaatctggtgtccaaggtgttggtcgatctgaggaggaccatccaggtctatatcagagcagggacgttctccgggtcggagatcaacaataagtatgacaacatcaggctgcttccagaggatcttagggacagcattcacatcctaacaaccacagacaaaccagatcctgctgcaactgtccaactggcagcggatgacacatcgcctcttgactgtgctgtgtaccatctcatctggggcgatacctggggcatgacgaggaaggtgcacggaagaattctcattccatcctctcgtgtgacaactggattcgcaggggtcatggtgacagatcactacgcaactgtggcagtattggagcttgctggtgctgttaacctgtgggagaaaaagctcattgatcttgaacatagaaacaacctcgatgttccgttgagcatgaatcgtgtgttggcaggttctagactcgggacccgggggataagatgggctcaatggacgtcgcctgcaggcctagagatcgttgtcaggaaagtgagggcaaagctgtcatcaatctcgggaaaccctggaaatgccgccaaatggaggaggaactgccagaaggtcctcaaggccttgatgatctccctgtatgctcattcgggcgaggacatgatgaggaatgcaagcggccttgtcaaggtgaacatacatgggaccctttcctctgtcaccgctgtgtgcgacccgtcggcggagagcagagtgcagagggcacagttcctgttcctaaaagaaaaggctcccaatgggcctttcattctaaggagcaggttggagaggcgcataaatctcctctcagttgtgtccgacctgctcgggtgatcaagaagtattaagaaaaaccctcatgttcacgtggttagaggggttacacccatactacatatcaaggtctttgacaatagtgtatttcgtgagaatgccatgtttttctgggtctctgggtcggcatggcatctccacctcctcgcggtccgacctgggcatccgaaggaggacgtccactcggatggctaagggagagctcgaattcggtacgctgaaatcaccagtctctctctacaaatctatctctctctattttctccataaataatgtgtgagtagtttcccgataagggaaattagggttcttatagggtttcgctcatgtgttgagcatataagaaacccttagtatgtatttgtatttgtaaaatacttctatcaataaaatttctaattcctaaaaccaaaatccagtactaaaatccagatccccaattcactggccgtcgttttacaacgtcgtgactgggaaaaccctggcgttacccaacttaatcgcctgtttaaactatcagtgtttgacagggacctgcagcccggccgggggatccactagcagattgtcgtttcccgccttcagtttaaactatcagtgtttgacaggatatattggcgggtaaacctaagagaaaagagcgtttattagaataatcggatatttaaaagggcgtgaaaaggtttatccgttcgtccatttgtatgtgcatgccaaccacaggagatctcagtaaagcgctggctgaacccccagccggaactgaccccacaaggccctagcgtttgcaatgcaccaggtcatcattgacccaggcgtgttccaccaggccgctgcctcgcaactcttcgcaggcttcgccgacctgctcgcgccacttcttcacgcgggtggaatccgatccgcacatgaggcggaaggtttccagcttgagcgggtacggctcccggtgcgagctgaaatagtcgaacatccgtcgggccgtcggcgacagcttgcggtacttctcccatatgaatttcgtgtagtggtcgccagcaaacagcacgacgatttcctcgtcgatcaggacctggcaacgggacgttttcttgccacggtccaggacgcggaagcggtgcagcagcgacaccgattccaggtgcccaacgcggtcggacgtgaagcccatcgccgtcgcctgtaggcgcgacaggcattcctcggccttcgtgtaataccggccattgatcgaccagcccaggtcctggcaaagctcgtagaacgtgaaggtgatcggctcgccgataggggtgcgcttcgcgtactccaacacctgctgccacaccagttcgtcatcgtcggcccgcagctcgacgccggtgtaggtgatcttcacgtccttgttgacgtggaaaatgaccttgttttgcagcgcctcgcgcgggattttcttgttgcgcgtggtgaacagggcagagcgggccgtgtcgtttggcatcgctcgcatcgtgtccggccacggcgcaatatcgaacaaggaaagctgcatttccttgatctgctgcttcgtgtgtttcagcaacgcggcctgcttggcctcgctgacctgttttgccaggtcctcgccggcggtttttcgcttcttggtcgtcatagttcctcgcgtgtcgatggtcatcgacttcgccaaacctgccgcctcctgttcgagacgacgcgaacgctccacggcggccgatggcgcgggcagggcagggggagccagttgcacgctgtcgcgctcgatcttggccgtagcttgctggaccatcgagccgacggactggaaggtttcgcggggcgcacgcatgacggtgcggcttgcgatggtttcggcatcctcggcggaaaaccccgcgtcgatcagttcttgcctgtatgccttccggtcaaacgtccgattcattcaccctccttgcgggattgccccgactcacgccggggcaatgtgcccttattcctgatttgacccgcctggtgccttggtgtccagataatccaccttatcggcaatgaagtcggtcccgtagaccgtctggccgtccttctcgtacttggtattccgaatcttgccctgcacgaataccagcgaccccttgcccaaatacttgccgtgggcctcggcctgagagccaaaacacttgatgcggaagaagtcggtgcgctcctgcttgtcgccggcatcgttgcgccacatctaggtactaaaacaattcatccagtaaaatataatattttattttctcccaatcaggcttgatccccagtaagtcaaaaaatagctcgacatactgttcttccccgatatcctccctgatcgaccggacgcagaaggcaatgtcataccacttgtccgccctgccgcttctcccaagatcaataaagccacttactttgccatctttcacaaagatgttgctgtctcccaggtcgccgtgggaaaagacaagttcctcttcgggcttttccgtctttaaaaaatcatacagctcgcgcggatctttaaatggagtgtcttcttcccagttttcgcaatccacatcggccagatcgttattcagtaagtaatccaattcggctaagcggctgtctaagctattcgtatagggacaatccgatatgtcgatggagtgaaagagcctgatgcactccgcatacagctcgataatcttttcagggctttgttcatcttcatactcttccgagcaaaggacgccatcggcctcactcatgagcagattgctccagccatcatgccgttcaaagtgcaggacctttggaacaggcagctttccttccagccatagcatcatgtccttttcccgttccacatcataggtggtccctttataccggctgtccgtcatttttaaatataggttttcattttctcccaccagcttatataccttagcaggagacattccttccgtatcttttacgcagcggtatttttcgatcagttttttcaattccggtgatattctcattttagccatttattatttccttcctcttttctacagtatttaaagataccccaagaagctaattataacaagacgaactccaattcactgttccttgcattctaaaaccttaaataccagaaaacagctttttcaaagttgttttcaaagttggcgtataacatagtatcgacggagccgattttgaaaccacaattatgggtgatgctgccaactcgagagcgggccgggagggttcgagaagggggggcaccccccttcggcgtgcgcggtcacgcgcacagggcgcagccctggttaaaaacaaggtttataaatattggtttaaaagcaggttaaaagacaggttagcggtggccgaaaaacgggcggaaacccttgcaaatgctggattttctgcctgtggacagcccctcaaatgtcaataggtgcgcccctcatctgtcagcactctgcccctcaagtgtcaaggatcgcgcccctcatctgtcagtagtcgcgcccctcaagtgtcaataccgcagggcacttatccccaggcttgtccacatcatctgtgggaaactcgcgtaaaatcaggcgttttcgccgatttgcgaggctggccagctccacgtcgccggccgaaatcgagcctgcccctcatctgtcaacgccgcgccgggtgagtcggcccctcaagtgtcaacgtccgcccctcatctgtcagtgagggccaagttttccgcgaggtatccacaacgccggcggccggccgcggtgtctcgcacacggcttcgacggcgtttctggcgcgtttgcagggccatagacggccgccagcccagcggcgagggcaaccagcccggtgagctctagtggactgatgggctgcctgtatcgagtggtgattttgtgccgagctgccggtcggggagctgttggctggct

SEQ ID NO: 8 - DNA, 18458 bp, Artificial sequence
  Synthetic nucleotide
ggtggcaggatatattgtggtgtaaacaaattgacgcttagacaacttaataacacattgcggacgtttttaatgtactggggtggttttggtaccgggccccccctcgaggtcgacggtatcgataagcttgatatcgaattcctgcaggcctcctaggactagttcccaggtcaacatggtggagcacgacactctcgtctactccaagaatatcaaagatacagtctcagaagaccagagggctattgagacttttcaacaaagggtaatatcgggaaacctcctcggattccattgcccagctatctgtcacttcatcgaaaggacagtagaaaaggaagatggcttctacaaatgccatcattgcgataaaggaaaggctatcgttcaagatgcctctaccgacagtggtcccaaagatgagacttttcaacaaagggtaatatcgggaaacctcctcggattccattgcccagctatctgtcacttcatcgaaaggacagtagaaaaggaagatggcttctacaaatgccatcattgcgataaaggaaaggctatcgttcaagatgcctctaccgacagtggtcccaaagatggacccccacccacgaggaacatcgtggaaaaagaagacgttccaaccacgtcttcaaagcaagtggattgatgtgatatctccactgacgtaagggatgacgcacaatcccactatccttcgcaagacccttcctctatataaggaagttcatttcatttggagaggagagacccatatatttcataaagggtcatctggtctgtatcacatagctgattagtcctcatgatgctgggcaagaatgcttgtgggaagatccgcccgcaacctgctcaataacacatgatcaggaagtgacacaatctgcgactattaacaaaaacccaaccacctctaaccttcttcatctcatccttcataatggcaaacatcaacatcccggatgacctggtatccaagtactcggaagatgtaaaggatctcacacaaaaggcaggtagcattccaagttctaaatcattgatcccccaaaccgcattcacccctgatgaattgaggagatctttgaagttctggcaagtgaccaccaaagtggcagcaactgtagcatccgactggactaatttggcgactgcattaaccaacgggacattcagtgcgacgcatctcaggacactgtgtgagctggcattcaacctcaaggccccaactggaacaggcacaatcttcattcatgagattgctgacccgtggaaagggtgcattgatgctaatgctcccgacactgtagcgatacctgcgactgacagtgatgttaatatgtctactgtcagctcaggttctaccgcctctgggacggaagatgcagaggtcaaaatgaaggcgatctcattcctatgctgcacattgctcaggctgtctgtgaaagagccgtcacacatcatgcaagcgatcaacagcatacgacagaggttcggctccctgtacggtgtctcctcccctaccttgaatagtgttacattcaacaggagccaactttctcgcatcaagcaaggcattgaaacatatccctctgctcggggtacagtattctattacaccaggtatgccgatgtcacgcatgggtatactaccaaagagtatgggatatgcaggttcctactgttccaacaccttgaacttgaggggatgcacatctataagatgctcttagctctacttggcgagtggtcaacagttccaattgggctcttgctcacctggatcaggaacccgaggtctaagttggccataaatgagattgtcaagatagtcaaggagcttgataagccggaagtggacaagggctggaaatatgctcggatggtcaataacacattcttccttgatctatcctctagaaggaacacttacctatgtgctgtgttggcctccctgaacaagaagaacgttccgcaaggatcaggggaatatgctgaccccactaacatagctgtgatcaagaatatggaccaatctgtgaaatcacaggttgccacagatgtgaccctgattgagagaatttatgagaaatatctggtgtctgcaggtggagacgaggcaggaacagcatatgctctgtctcgcggagttaagagatcctcgcctcaaagtcaggaaggcggtcaatccggccagtctggaggaacacccatggaagtcgacggagcatctggtagaggagcagcaggtccggccccgaagaagacacgatcaggcttatagatctcagctcggtatcaatagcctacatgagaagatgtaccacgtgccatacgctcctgaacctatgtgtgatcccaatggaccaacaataatagcagagtctgtcataaccctgccgttttaatcaattatcatgtgcttcccatgttgttgtttcaatctattaagaaaaacccaacctcaggttcagagcaccagtggtctagtggtagaatagtaccctgccacggtacagacccgggttcgattcccggctggtgcagtttgactgaactggaacatgttttagagctagaaatagcaagttaaaataaggctagtccgttatcaacttgaaaaagtggcaccgagtcggtgcttcagagcaccagtggtctagtggtagaatagtaccctgccacggtacagacccgggttcgattcccggctggtgcacctcattgtcgttggttgaagttttagagctagaaatagcaagttaaaataaggctagtccgttatcaacttgaaaaagtggcaccgagtcggtgcttcagagcaccagtggtctagtggtagaatagtaccctgccacggtacagacccgggttcgattcccggctggtgcaatctcagctcggtatcaatagcctacatgagaagatgtaccacgtgccatacgctcctgaacctatgtgtgatcccaatggaccaacaataatagcagagtctgtcataaccctgccgttttaatcaattatcatgtgcttcccatgttgttgtttcaatctattaagaaaaacccaacctcaggatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaaatctcagctcggtatcaatagcctacatgagaagatgtaccacgtgccatacgctcctgaacctatgtgtgatcccaatggaccaacaataatagcagagtctgtcataaccctgccgttttaatcaattatcatgtgcttcccatgttgttgtttcaatctattaagaaaaacccaacctcaggatgaatcgttactcccgtcgttctagacatcccaaccctcctgtccccaatcaggaagaacccgagcgcgacccgaatcacatagatcaagacctcgcagacttggctcaacccttggtcctaaaagaaagacacgcagtaatggcaccaactcagcccagccttagcgatgtgatcaatgaggaaagacaggctcccatcacgtttggtaatcctccagaggtcatggcgaacgctaggctatcagcgctgggttatgacaatctcacggagagggagaagaggatccttgctgtaggaatcagatgcggagaggctgccaaagactatcattcgctcactaccaccaaaaagtggattgaggatgagctgaagtctcaaatggtggctctcgcatcatcaacaaggactctcactgaggctgcatcacttcacacgacctttgcaatgctgcattctccatccattaaaagaaaggcagaagcaatgtctcacatatctcaaggcgaagagagcattgacatttctaagctgaataagactggaatggaggacatctgggtggcaatggagagtgagtcaaaagaggatgcagtagacacgtatctgagaaacattctggaagtggacccgacacaattctatgcaatagacggctggggcctctacctggacttcattcccacttggcattatatagctgcagggaagaattcggcgcaatttaagacgagttatgctgacgagattgtagagcagcgtgctgcattcgagagggtcctctctaagcgccctagggttgagatctaagcctgtatgtcactccactccccaataatgtccacgtcagcctcatgacaccctgctattaagaaaaacccaacaatggagccctctgcagagatccccctcgggaaaggcaaggaaaccgggagaagccctctgatcgcaacaaggaggaagaccgggctggtcaaagccaagataaagaccaattcatccttctctatgttcaggagagcagatactatgcgaacccatgcacagttcaccaacattactgtggggtggaagtcgctttgtcctatcactgcagaaggacaggtcaatgtagccatataccatgagagcacccgagtccctgttctcaatctatggagtcctgtgtcctctagttggaagcacctggcaacaggcagtctaggctttgttagtctgaaccactgtccatatgtggttgaaggaagaatatcgggattcgagggagaagaagcagggttggtgacacttacacttcatcttgacactggattggagagtgatgacatccagaagtgtcgtttattttctgaacatccggagttagcgggtggatcactatatttatacacttcctacacaagtagccccataccggacagcaatctccctgccttaaggactgagatcgatgacactatcgctgcaatttggaggatgagtggagggaagatgtctatcaccacaagccatgctctagggctgattgccaccttcagggttaaaatagagtctttacttggggaatacccggatgatgcattctctgctcaggttgagaatcttgtgaaggattttctccttgtttcatcgacagctgagataaccagatcttacaggacctgccttgcaaaagtgctgtcgtcgatgaccacgtatgacaagaagtactgggacaccatcataacaggtccctcctccatatgacactcgccatcgcagcagctggagctcatattcccatcatcaagcacatctgcaagaataaaaatccgctcccacccttcattcatcctcacaaccaccctccgcccttgttaagaaaaacccaacatggatgctcctatcaatccttcctctcccacccggtcttcctatgagacagccctagagaggcaatggttgatcaaggatgaaccgtcacaccatgttgacttgggatgtcacttcacattagagatcaaagcattctcacccaaaatgattgagttgctggagactggtagcctgactctaggatccgtgtatcaagggctgtacaatttgatcaaggacaaagactgcaaggggctggagctagatcgggacaccacccatgggaacaaattgcatgtcttggcggcagctttcctgtgtgtgctcaggaagtatgagaaccaggtgcggttttacaatgtcaaaaccaaggactctctgacgggcgaaatccagtatatgtctagggtacacatcggagatgataacttggggctcttcgatccagacacctacaggatgataggagggtcatttccggaagggatctaccgactgaacctccgcacacaactcacacccaggggagaagatgacggtctggaaatcataatgggtaccattctaaccgtccccgtgcgcggtcctgctgaccgacagcctctcggagtattgatggacagacaactaagacagttcccggtagggaccaacccttggtcgtccttgggggaggcgaaacctaaagggagatcgttgtcactcctgcgtggaatccttatgtaattctgcatatcacctggttgatatccaataagaatgccactgcccagcattaccccacgattaagaaaaacccaacttacacagacgcaatcatgtctctcattcacctcatctacttccccaccatcctgtctctcatcctaggtacagagggcacaccagcattgacttatggcaacctggctgacaatcagcatggaaatgaggagcaaggggatgttgatatgtatcctctatatgagtgcagtaagaagggtgcaggagtctctagcaataactggtacggagcctgccgaggagcttgctctatgaccaaaaatactactgatgtccacatggagatatattacaggaatgacagtgtcggttggatagatgtattgtctatccagactacagaaatctggaagcactcacatgtaacctggtacggagagtgtgaacacaatacaaaatatgggagctcgaacacagcacctgtctccgtcattctggagcggttggagttccttcacaatgggatgtcatcatggccatacggtcatagcattcatgttcatgacactatctcaagccctgactgtagctattttggtgatgtgtccagatccggatggagattgataatatctaaacgtgctcttgaactgaaatcagatattgcaggagagggctatattgtagaccccgatgtggggtatgtatatcctgttgcggacggaattgggaaaggtaggacctggcatgtatggaaagctagctctgttccgtcttctggctgctacttcaaatcggcaggaattactaattgtaccctactaatggatacattcctctattcatgtccaagcctcaatatagccttctcagctcgggtcgggaaacacataaagagcacgtgcgtcggagatatgaatatatcaacagacggtgtgacttatcagactctcgggagcacggatcaagggtcgatatccacacagttggtatcattatggcatcaatcacaggaagctctcatacagcaacttattttgactatcaacgaggccctaggaaaaattgagacaagctactgtgagtccacttgtgatttgaccgagatgttaatctcccagcataccgagcacccgatggtaattgagacgcctgtcgggccatggctaccatacagcatagacggtaaagtttctgttctcccttgtcaaggtggtcaggatctggtggtgatcaagcctatagagatctgcgcttctccattcatgctcaaagttaaatcactcaagtctctagacacctactggtgggtccctacagatagccacgtggctccgttcacgcaatgcagcaacagggacgatgaagaaatgatcatcaagatacaacggaagaagccattgatatttgaattctggagaggagcctataagcttgattacccgtataactctagtggaatctggctggataaccctggaggtcatattcatagaagctctaaatggttccctacgttggatagtctatcctacagttctcctataaacttgccactgatcagcaaaggtataaggaaacatattcaagccatcgtcagtaccaccgagatcacatctggaggtaacgttacatcatggacccactcgatttcgactattacagaggccatgttcgactcagctgcgatagccgcgggtcgggtcgtgatgtggtggtacacactagaagaaagcgtcaaaaggtatctgactgtagcgttcggtctagtactaactgtcatcaccatttgggctatgtcaaaattattcttccgatcgtcgccaacatatgccagggcgcccacaaatatagaatgggtaaggtcatagacatcgcagtgtccacccacctcatctccatggagcccgatccacaataaaatgccatctatgtatcaaattaatcctatatgtatattaagaaaaacccaattccaatggatccagattatcctgatcttgatcccgagtctcttgatgttctcaacagcctacaagaagggtacgatgaggaggaagaagaggacagcggagtgtcactcaatggaatgggggactaccaccttaagtcagcactcaggtcgtacacagatatgatgaagcacccaatcttcaagaaggagttctccaaggcagtgattaactttggtatatctcacgactcgatgatgtcccagatagaaacaatgttctacatcctgaagagtactccattgtcaatgcaccttgggaccctttatggtgatctcttcactcgtcagagcacgctcggccacagcgaagatgtatttgacattatccaagcagagatacgaactcttcatcctcacctcctcctagagatgacacctgccaaggtcgaggagatgattctgattgctgacaggaagacatatgatgagagggcagcatctgcgttctggtccactctcataacaataaagaattacattccagcttggacagagaagggggcttgtactctggattggcctagtgtgtctctagacaagaagagtgggtacgtgaaagtgatggcaggtaaagatactaccatctacattgggactgatatttgtgtgatagagaagtatcctacagtaaaatgggctccactttcataccttttaaatggagctgacaaggtggcagaaaggctgaatgtgcgttactatagttgtctttgtgatcagcttgatataccagataggataacacttgatatgcttgatgaaatcatcaaagtgggggatgattgtcttcgagagatgggcaatgacggatacaatgtaattggatcctatgaggctttgcttgcaggaataatccaaaggagggacaaccagggactaataccggacagagatttgttgtggagaactactatggaagaatttacatccagccccggcaggaagtatctagaagagtggagcacaatgtttagttcattaagccctgaacagatagcctgtgctcatggactgtataggatctgggggcacccaatagttgatattcttggagggataaagaagatgcaagaggtggcatctgtgaagaagaatcctagacagagtgtactggatgaaataaggaggcaattcaaagaaatgttttttacctcatatcataggatacacaagcactaccctctacatacaatcttgaacagcctggataattcctatatactcaactgtctaaaggataacttgacgatcaacacatcagcaatctcctataacttccaggattgggactgtgtgatggtggagaaaaacttcgaagtgcctttctcttggaacctggttcacaatttgaaagataaagctatttccccgaacaggcaggagatttaccagacgatatcaactcgtggtacaatattcgggtctcagaaccgcagagggatactgaaatcactcacgatggagactgtccaactccgagactttcttcaaggcataaatgacaacgggttggatgacatagacaaaattattggggtctatcctaaagagcgtgagctgaagataaaggctagactgttctccctcatgtcattccggctcagattgtactgtgtgtcaacagaggcattattaggggacaaaatactcaagtatttcccgcaaatcacaatgtctcttgatatgctcacgatgataaagaagatgttcaaagtgtcctcccaaactactagagaggatgattcagtcacagtcatattcaatcttgacttcataaagtggaatctccagatgagacgtaatatatgtgagcctgtcttctcacaattaggtaaactctttggcatggagaatctgttcaataggactcatgagaccttccgagatagcctgatatacctctgttcgggggaaggagtactgtcagccgatccagtctacggagtattcccggacggtacatgggcatgggcgggtgatgagtcggggaaggaagggcttcgtcagaagggttggacaattctgactgtcgttacaatcatgttgattgcaaagcgacacaatgttgatgtatcactaatgggaggaggagacaatcaagtccttggtatcaccatatcaggagtgaccagagatcttcaaggagagctgattacagaatcggcctcactcgcccgaaggacaatcaagcaatttaccgaggacttattaaccactttctccgatctggggctccctttaaaagcatctgaaacatgggtcagtgactctctcttcatgtacaacaaacacatgttttacaagggaattcctctcagatcacctctgaaagccatctcaaggatcttcccgctggcaaacgacacaattatgaccatagacaacatgatcaataatatatcatcaggggtcaaagcagcctgtatgaaggagagacacggaattccacttgtattcatgaaaacactagcatataggaaggttgccgagggagcactgatcctacatccattgacgacttgcttcaggacaccaacattgcctgaaaatggaactgtgacgcgtgacggacggaagcgatcagtgaaggtgactaggaatcagctgagatcattctttacgttatgtatggtgggatcttccactatgggactacctggtactatacatctccctgatatgatcatgaggggattccctgatccattgaccagctacctgtcattcatttcagaattggttcccaagatcatggacccaagactagcgagcatcatcgcaacatcgggtcatatgagcatcaatagaatgacagagtatgcgaaacttgtggaggatcctgtatctataaatcacgacgcccccacacatggactgaatgagatccgacagttatctcgagacttcttgatgaacacgaccctggcagccaacacccatcttaagtcccttttcaccctcctagacagacagagagaaaaagaattctacgacgcactatgttctgctgaggaactggatgtgaaggtgctgcacgaagtagccggagcatcactattcggatatacaaacggcattgcctctcgtattgaccagacaagaacggtgagagcgttaaacgagacagtcaacgttatgaaaaggctagctgatgcagaggcaagatatattgcatatctgttcgctagggacatacataagcatgacctcatcccggatcagtgttccagagtgactgcggacaggtttgtttctgcttctacctttgatatatatataataattatcattaattagtagtaatataatatttcaaatatttttttcaaaataaaagaatgtagtatatagcaattgcttttctgtagtttataagtgtgtatattttaatttataacttttctaatatatgaccaaaacatggtgatgtttaggtatcgatattattcatggaagaagaaggttttaggggtaacagtacctcacccgattgagatgtgtcaagtcacatcggcgacccaaactgcattcgaggatgctgttatatgttggagtgataatctgagcggatcgagaatctacaccacaatgggtctagggaagatatatcaaggttcctacaccaaggaaaggttcaaggccacagacattgctgccgcgtatgggaacgaggacatcctgacaaaagcagtcaggctgcagaagctaattaactggagatatgatgaagggagcagctttgctaggataattcaacttgctctcagagccatcactgatgcaagcactgacggattccatagatctaaggaggagatcaaaggggaattcgatcatagaaggggcatatcaggagatatcagcggaggaattccaaatttccttgtcacacccacctcgcatttctcgtgtaccacatcctcttggatctcacatagcaggggagggaaaaacgagaatatccatttccagagtgtactgatcaacttattatacagagccatggtctatcgaggctcattgcccggtctaccagagatggtctggtattcaaaagagagatgcagccattgcattattgatattgtggaaccgtctccagaacgaacaaccccagctttgtcagagatgccatcagcacctaacaatccttttgcattcatagagtcagtaaatgtgaagttggattatcatcaccaggttgagatagaaaaaggaatggaagagaccaacctactaaattctgagtggggaggctcatctttaacaagtgtggatgaggctggactggtcttgtttctgatgctgattggatcccgtcaagtgtcagaatcgtttttgttattgatgagggagaagatagaacctcgaccgctactgcaagcagcattgaacagagtcatccttgtcaggaaactcggattggataggttgttccctataagaaacaccaaatgcatcaatcttatactggggtctgacagaggaatcactgcatgccatgacggctttggcctggaactgcacgggggatcctgggagaaagggatagagtgtgacacctcctttctttacaatgatactcacctcaagccctgtaaattgcatgttaaccttcatcttcaaaatgtgcctctccagctggccttggcgcatgcttcctataatcctgatgtttgtgagtgtcttgagtgcagagccatcattgaagatagagtctctaagaggatggtagggcaatataagagctggagatgccagtatcatgcggtcctgtccttctctccaatcatcatgagggtccacagtgagaggataatcaaaaatgagggagctatatatgaaggggatctgcatgtcccttatgtcgaggagatcattgctctggagaactcacaaaaattggcaatagatgtgacgtcatggagactgccgctgctgatgcacgcaacatgggagtctatcctgcctcagctgtatattactatgaaggcctgcctggtttgtcttcccttggagggtgtgattgtggatgatgacctggcactgatcaatctggtgtccaaggtgttggtcgatctgaggaggaccatccaggtctatatcagagcagggacgttctccgggtcggagatcaacaataagtatgacaacatcaggctgcttccagaggatcttagggacagcattcacatcctaacaaccacagacaaaccagatcctgctgcaactgtccaactggcagcggatgacacatcgcctcttgactgtgctgtgtaccatctcatctggggcgatacctggggcatgacgaggaaggtgcacggaagaattctcattccatcctctcgtgtgacaactggattcgcaggggtcatggtgacagatcactacgcaactgtggcagtattggagcttgctggtgctgttaacctgtgggagaaaaagctcattgatcttgaacatagaaacaacctcgatgttccgttgagcatgaatcgtgtgttggcaggttctagactcgggacccgggggataagatgggctcaatggacgtcgcctgcaggcctagagatcgttgtcaggaaagtgagggcaaagctgtcatcaatctcgggaaaccctggaaatgccgccaaatggaggaggaactgccagaaggtcctcaaggccttgatgatctccctgtatgctcattcgggcgaggacatgatgaggaatgcaagcggccttgtcaaggtgaacatacatgggaccctttcctctgtcaccgctgtgtgcgacccgtcggcggagagcagagtgcagagggcacagttcctgttcctaaaagaaaaggctcccaatgggcctttcattctaaggagcaggttggagaggcgcataaatctcctctcagttgtgtccgacctgctcgggtgatcaagaagtattaagaaaaaccctcatgttcacgtggttagaggggttacacccatactacatatcaaggtctttgacaatagtgtatttcgtgagaatgccatgtttttctgggtctctgggtcggcatggcatctccacctcctcgcggtccgacctgggcatccgaaggaggacgtccactcggatggctaagggagagctcgaattcggtacgctgaaatcaccagtctctctctacaaatctatctctctctattttctccataaataatgtgtgagtagtttcccgataagggaaattagggttcttatagggtttcgctcatgtgttgagcatataagaaacccttagtatgtatttgtatttgtaaaatacttctatcaataaaatttctaattcctaaaaccaaaatccagtactaaaatccagatccccaattcactggccgtcgttttacaacgtcgtgactgggaaaaccctggcgttacccaacttaatcgcctgtttaaactatcagtgtttgacagggacctgcagcccggccgggggatccactagcagattgtcgtttcccgccttcagtttaaactatcagtgtttgacaggatatattggcgggtaaacctaagagaaaagagcgtttattagaataatcggatatttaaaagggcgtgaaaaggtttatccgttcgtccatttgtatgtgcatgccaaccacaggagatctcagtaaagcgctggctgaacccccagccggaactgaccccacaaggccctagcgtttgcaatgcaccaggtcatcattgacccaggcgtgttccaccaggccgctgcctcgcaactcttcgcaggcttcgccgacctgctcgcgccacttcttcacgcgggtggaatccgatccgcacatgaggcggaaggtttccagcttgagcgggtacggctcccggtgcgagctgaaatagtcgaacatccgtcgggccgtcggcgacagcttgcggtacttctcccatatgaatttcgtgtagtggtcgccagcaaacagcacgacgatttcctcgtcgatcaggacctggcaacgggacgttttcttgccacggtccaggacgcggaagcggtgcagcagcgacaccgattccaggtgcccaacgcggtcggacgtgaagcccatcgccgtcgcctgtaggcgcgacaggcattcctcggccttcgtgtaataccggccattgatcgaccagcccaggtcctggcaaagctcgtagaacgtgaaggtgatcggctcgccgataggggtgcgcttcgcgtactccaacacctgctgccacaccagttcgtcatcgtcggcccgcagctcgacgccggtgtaggtgatcttcacgtccttgttgacgtggaaaatgaccttgttttgcagcgcctcgcgcgggattttcttgttgcgcgtggtgaacagggcagagcgggccgtgtcgtttggcatcgctcgcatcgtgtccggccacggcgcaatatcgaacaaggaaagctgcatttccttgatctgctgcttcgtgtgtttcagcaacgcggcctgcttggcctcgctgacctgttttgccaggtcctcgccggcggtttttcgcttcttggtcgtcatagttcctcgcgtgtcgatggtcatcgacttcgccaaacctgccgcctcctgttcgagacgacgcgaacgctccacggcggccgatggcgcgggcagggcagggggagccagttgcacgctgtcgcgctcgatcttggccgtagcttgctggaccatcgagccgacggactggaaggtttcgcggggcgcacgcatgacggtgcggcttgcgatggtttcggcatcctcggcggaaaaccccgcgtcgatcagttcttgcctgtatgccttccggtcaaacgtccgattcattcaccctccttgcgggattgccccgactcacgccggggcaatgtgcccttattcctgatttgacccgcctggtgccttggtgtccagataatccaccttatcggcaatgaagtcggtcccgtagaccgtctggccgtccttctcgtacttggtattccgaatcttgccctgcacgaataccagcgaccccttgcccaaatacttgccgtgggcctcggcctgagagccaaaacacttgatgcggaagaagtcggtgcgctcctgcttgtcgccggcatcgttgcgccacatctaggtactaaaacaattcatccagtaaaatataatattttattttctcccaatcaggcttgatccccagtaagtcaaaaaatagctcgacatactgttcttccccgatatcctccctgatcgaccggacgcagaaggcaatgtcataccacttgtccgccctgccgcttctcccaagatcaataaagccacttactttgccatctttcacaaagatgttgctgtctcccaggtcgccgtgggaaaagacaagttcctcttcgggcttttccgtctttaaaaaatcatacagctcgcgcggatctttaaatggagtgtcttcttcccagttttcgcaatccacatcggccagatcgttattcagtaagtaatccaattcggctaagcggctgtctaagctattcgtatagggacaatccgatatgtcgatggagtgaaagagcctgatgcactccgcatacagctcgataatcttttcagggctttgttcatcttcatactcttccgagcaaaggacgccatcggcctcactcatgagcagattgctccagccatcatgccgttcaaagtgcaggacctttggaacaggcagctttccttccagccatagcatcatgtccttttcccgttccacatcataggtggtccctttataccggctgtccgtcatttttaaatataggttttcattttctcccaccagcttatataccttagcaggagacattccttccgtatcttttacgcagcggtatttttcgatcagttttttcaattccggtgatattctcattttagccatttattatttccttcctcttttctacagtatttaaagataccccaagaagctaattataacaagacgaactccaattcactgttccttgcattctaaaaccttaaataccagaaaacagctttttcaaagttgttttcaaagttggcgtataacatagtatcgacggagccgattttgaaaccacaattatgggtgatgctgccaactcgagagcgggccgggagggttcgagaagggggggcaccccccttcggcgtgcgcggtcacgcgcacagggcgcagccctggttaaaaacaaggtttataaatattggtttaaaagcaggttaaaagacaggttagcggtggccgaaaaacgggcggaaacccttgcaaatgctggattttctgcctgtggacagcccctcaaatgtcaataggtgcgcccctcatctgtcagcactctgcccctcaagtgtcaaggatcgcgcccctcatctgtcagtagtcgcgcccctcaagtgtcaataccgcagggcacttatccccaggcttgtccacatcatctgtgggaaactcgcgtaaaatcaggcgttttcgccgatttgcgaggctggccagctccacgtcgccggccgaaatcgagcctgcccctcatctgtcaacgccgcgccgggtgagtcggcccctcaagtgtcaacgtccgcccctcatctgtcagtgagggccaagttttccgcgaggtatccacaacgccggcggccggccgcggtgtctcgcacacggcttcgacggcgtttctggcgcgtttgcagggccatagacggccgccagcccagcggcgagggcaaccagcccggtgagctctagtggactgatgggctgcctgtatcgagtggtgattttgtgccgagctgccggtcggggagctgttggctggct

SEQ ID NO: 9 - DNA, 22724 bp, Artificial sequence
  Synthetic nucleotide
ggtggcaggatatattgtggtgtaaacaaattgacgcttagacaacttaataacacattgcggacgtttttaatgtactggggtggttttggtaccgggccccccctcgaggtcgacggtatcgataagcttgatatcgaattcctgcaggcctcctaggactagttcccaggtcaacatggtggagcacgacactctcgtctactccaagaatatcaaagatacagtctcagaagaccagagggctattgagacttttcaacaaagggtaatatcgggaaacctcctcggattccattgcccagctatctgtcacttcatcgaaaggacagtagaaaaggaagatggcttctacaaatgccatcattgcgataaaggaaaggctatcgttcaagatgcctctaccgacagtggtcccaaagatgagacttttcaacaaagggtaatatcgggaaacctcctcggattccattgcccagctatctgtcacttcatcgaaaggacagtagaaaaggaagatggcttctacaaatgccatcattgcgataaaggaaaggctatcgttcaagatgcctctaccgacagtggtcccaaagatggacccccacccacgaggaacatcgtggaaaaagaagacgttccaaccacgtcttcaaagcaagtggattgatgtgatatctccactgacgtaagggatgacgcacaatcccactatccttcgcaagacccttcctctatataaggaagttcatttcatttggagaggagagacccatatatttcataaagggtcatctggtctgtatcacatagctgattagtcctcatgatgctgggcaagaatgcttgtgggaagatccgcccgcaacctgctcaataacacatgatcaggaagtgacacaatctgcgactattaacaaaaacccaaccacctctaaccttcttcatctcatccttcataatggcaaacatcaacatcccggatgacctggtatccaagtactcggaagatgtaaaggatctcacacaaaaggcaggtagcattccaagttctaaatcattgatcccccaaaccgcattcacccctgatgaattgaggagatctttgaagttctggcaagtgaccaccaaagtggcagcaactgtagcatccgactggactaatttggcgactgcattaaccaacgggacattcagtgcgacgcatctcaggacactgtgtgagctggcattcaacctcaaggccccaactggaacaggcacaatcttcattcatgagattgctgacccgtggaaagggtgcattgatgctaatgctcccgacactgtagcgatacctgcgactgacagtgatgttaatatgtctactgtcagctcaggttctaccgcctctgggacggaagatgcagaggtcaaaatgaaggcgatctcattcctatgctgcacattgctcaggctgtctgtgaaagagccgtcacacatcatgcaagcgatcaacagcatacgacagaggttcggctccctgtacggtgtctcctcccctaccttgaatagtgttacattcaacaggagccaactttctcgcatcaagcaaggcattgaaacatatccctctgctcggggtacagtattctattacaccaggtatgccgatgtcacgcatgggtatactaccaaagagtatgggatatgcaggttcctactgttccaacaccttgaacttgaggggatgcacatctataagatgctcttagctctacttggcgagtggtcaacagttccaattgggctcttgctcacctggatcaggaacccgaggtctaagttggccataaatgagattgtcaagatagtcaaggagcttgataagccggaagtggacaagggctggaaatatgctcggatggtcaataacacattcttccttgatctatcctctagaaggaacacttacctatgtgctgtgttggcctccctgaacaagaagaacgttccgcaaggatcaggggaatatgctgaccccactaacatagctgtgatcaagaatatggaccaatctgtgaaatcacaggttgccacagatgtgaccctgattgagagaatttatgagaaatatctggtgtctgcaggtggagacgaggcaggaacagcatatgctctgtctcgcggagttaagagatcctcgcctcaaagtcaggaaggcggtcaatccggccagtctggaggaacacccatggaagtcgacggagcatctggtagaggagcagcaggtccggccccgaagaagacacgatcaggcttatagatctcagctcggtatcaatagcctacatgagaagatgtaccacgtgccatacgctcctgaacctatgtgtgatcccaatggaccaacaataatagcagagtctgtcataaccctgccgttttaatcaattatcatgtgcttcccatgttgttgtttcaatctattaagaaaaacccaacctcaggttcagagcaccagtggtctagtggtagaatagtaccctgccacggtacagacccgggttcgattcccggctggtgcatggatggtaatccgcctgaagttttagagctagaaatagcaagttaaaataaggctagtccgttatcaacttgaaaaagtggcaccgagtcggtgcttcagagcaccagtggtctagtggtagaatagtaccctgccacggtacagacccgggttcgattcccggctggtgcaatctcagctcggtatcaatagcctacatgagaagatgtaccacgtgccatacgctcctgaacctatgtgtgatcccaatggaccaacaataatagcagagtctgtcataaccctgccgttttaatcaattatcatgtgcttcccatgttgttgtttcaatctattaagaaaaacccaacctcaggatggactataaggatcacgatggcgactacaaggatcatgacattgactataaggatgacgacgataagatggcacctaagaagaaaaggaaagtcggcattcatggcgttccggcagccgacaaaaagtatagcatcggcctcgatattgggacaaactctgtgggctgggcggtaattaccgacgagtacaaggtgcctagtaagaaatttaaagtgctcggaaacactgacaggcactctataaagaagaacctgatcggggcactgcttttcgactccggagagacggcggaggcgacgcgtctcaagcgtaccgcgcgccgcaggtacacaagaaggaagaataggatctgctacttgcaggaaatcttcagtaacgagatggcgaaggtcgacgatagtttctttcatcggttggaagaatcgttcctcgtagaggaggacaaaaagcacgagcgtcacccaatattcgggaatattgttgacgaggttgcctaccatgagaaatatcctacaatatatcacctccgtaagaagcttgtcgattcaactgataaggctgatctcagactcatctatcttgccctcgcacatatgattaagtttcgtggccacttcttgattgaaggcgacctcaacccggacaactcagatgttgacaagctttttatacagctcgtccagacatataaccagctgtttgaagagaatcccatcaatgcgagtggggttgatgctaaggccattttgtccgccaggttgtccaaatctcgcagactggaaaacctgatcgcacagcttcccggtgaaaagaaaaacgggctcttcggcaatctcatcgcactgtccctcggcctcaccccaaacttcaagtctaacttcgacctggccgaggatgcgaagctccagctgtcaaaagatacatacgacgacgatttggacaatctgcttgcgcaaataggcgaccagtatgcggacctgttcctggctgccaaaaatctgtcagatgcaatcctcctgtccgatatattgcgtgtgaacaccgaaatcacgaaggcaccgcttagcgcatccatgatcaagagatacgacgagcaccatcaggacctcacactcctcaaggcgcttgttcgtcagcagcttcccgagaaatataaggaaatttttttcgatcaaagcaagaatggatatgctggctatattgacggtggcgcttcgcaggaggagttctataaattcattaagccgattctggagaagatggacggaacggaggagctcctcgtcaagcttaaccgggaagacctgttgcggaagcagaggacttttgataacggctctattccgcaccaaatccatctgggtgagttgcacgcaatcttgagaagacaagaggatttctacccgttccttaaggataacagagagaagatagaaaaaatactgaccttcaggataccatactatgtgggcccactggcgcgcggaaatagtcgtttcgcatggatgactagaaagtccgaagaaacgatcacgccatggaattttgaggaagtggtcgacaagggcgcctctgcccagagcttcatcgaaaggatgaccaattttgacaaaaatctgcctaacgaaaaggtgcttccgaagcacagcctgttgtatgaatacttcacagtttataacgagctcactaaggtcaagtacgtcacggagggcatgcgtaagcctgctttcctgtctggtgaacaaaaaaaggcgattgtggacctccttttcaagacgaaccgtaaagttactgtgaagcaactgaaagaggattactttaagaaaattgagtgcttcgacagtgtggagatttccggtgtcgaggaccggtttaacgccagcctgggtacgtatcatgacctgcttaaaattatcaaggataaagatttcctggataatgaagagaacgaagatatactggaggacattgtgttgactttgaccctcttcgaggacagagagatgattgaggaaagactgaagacctacgcacacctttttgatgacaaggtcatgaaacaactcaagcgccggcgctatactggctggggccggctttctcgcaagctcatcaatgggattcgggataagcaatcaggcaagacaattttggacttcctcaaatccgacggattcgcaaataggaattttatgcagctgatacatgacgactctttgacattcaaagaagacatacagaaggctcaggtctccggccaaggagattctttgcacgagcatatcgctaacttggcaggtagccccgccataaaaaagggcattcttcaaacggtaaaagttgttgacgaactcgtgaaggttatgggccgtcataagccggaaaacattgttattgaaatggctagggaaaatcagacgacccagaagggacagaaaaatagcagggagcggatgaagagaattgaagagggaattaaggagcttggatctcagattcttaaggagcaccctgtggagaacacccaacttcagaatgaaaagctctacctttactaccttcaaaacggccgggatatgtacgtcgatcaggaacttgacattaaccggttgagcgattatgacgttgaccatattgtgccccaatctttccttaaagacgactctatcgacaataaagtgctgacgcgcagcgataaaaatcgcggtaagtcggataatgtcccgtcggaagaggtggttaaaaaaatgaagaactattggaggcaactcctgaatgccaagctgatcactcagaggaaattcgacaatctcaccaaggcagaaaggggtggacttagcgagctcgacaaggccggttttatcaaaagacagctggtggagacacgccaaatcaccaaacacgttgcccagatcctggattcgaggatgaacacgaagtatgacgagaacgacaagttgattagggaagtcaaggtcatcactttgaagtccaagctggtgagcgactttcgcaaagacttccagttttacaaagtcagggaaattaataactaccaccacgcccacgacgcctaccttaacgccgtggttggcacagcactcatcaagaaataccctaagctcgaatctgagttcgtctatggcgactataaggtctacgacgttagaaaaatgatcgcgaaatctgagcaggaaataggcaaggcaactgccaagtacttcttctattccaatatcatgaacttttttaagacggagattaccctggcgaatggtgagatccgcaagcgccctttgattgagacaaacggagaaacaggagagatcgtatgggacaaagggcgggactttgctactgttaggaaggtgctctctatgccacaagttaacattgtcaaaaaaactgaagtgcagacaggtgggtttagcaaggaatctatcctgccgaagaggaactctgacaagctgatcgcccgcaagaaagattgggatccgaaaaagtacggaggattcgactcccccacagttgcgtactccgtgcttgtcgtggccaaagtggagaagggcaagtctaagaagctcaagagcgtcaaagagttgttggggatcacgattatggagcggtcgtctttcgaaaagaatccgatagattttctcgaggccaagggttataaagaagtcaagaaggatcttatcatcaagctccctaagtactccctctttgagcttgaaaacggacggaaaagaatgctggcttcagcgggtgaacttcagaagggtaatgaactcgctctgccctcaaaatatgtgaatttcctttacctggcatcacactatgagaagcttaaggggtctccagaggacaacgagcagaagcaactgttcgttgaacaacacaagcactaccttgacgagattatcgagcaaatcagcgagtttagcaagcgcgttatactggcagacgcaaatcttgataaggtccttagcgcctacaacaagcatagagacaaacccatccgggagcaggccgagaacattattcatctcttcaccttgacgaatcttggggccccggccgcgttcaagtacttcgatactaccatagacagaaagcgctatacatcgacaaaggaagttcttgacgccacgctgatccaccaaagtataacaggcctctatgagacacgcatcgacctttcgcagttgggcggtgaccgccccaaaaagaagaggaaagttggcgggtgaatctcagctcggtatcaatagcctacatgagaagatgtaccacgtgccatacgctcctgaacctatgtgtgatcccaatggaccaacaataatagcagagtctgtcataaccctgccgttttaatcaattatcatgtgcttcccatgttgttgtttcaatctattaagaaaaacccaacctcaggatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaaatctcagctcggtatcaatagcctacatgagaagatgtaccacgtgccatacgctcctgaacctatgtgtgatcccaatggaccaacaataatagcagagtctgtcataaccctgccgttttaatcaattatcatgtgcttcccatgttgttgtttcaatctattaagaaaaacccaacctcaggatgaatcgttactcccgtcgttctagacatcccaaccctcctgtccccaatcaggaagaacccgagcgcgacccgaatcacatagatcaagacctcgcagacttggctcaacccttggtcctaaaagaaagacacgcagtaatggcaccaactcagcccagccttagcgatgtgatcaatgaggaaagacaggctcccatcacgtttggtaatcctccagaggtcatggcgaacgctaggctatcagcgctgggttatgacaatctcacggagagggagaagaggatccttgctgtaggaatcagatgcggagaggctgccaaagactatcattcgctcactaccaccaaaaagtggattgaggatgagctgaagtctcaaatggtggctctcgcatcatcaacaaggactctcactgaggctgcatcacttcacacgacctttgcaatgctgcattctccatccattaaaagaaaggcagaagcaatgtctcacatatctcaaggcgaagagagcattgacatttctaagctgaataagactggaatggaggacatctgggtggcaatggagagtgagtcaaaagaggatgcagtagacacgtatctgagaaacattctggaagtggacccgacacaattctatgcaatagacggctggggcctctacctggacttcattcccacttggcattatatagctgcagggaagaattcggcgcaatttaagacgagttatgctgacgagattgtagagcagcgtgctgcattcgagagggtcctctctaagcgccctagggttgagatctaagcctgtatgtcactccactccccaataatgtccacgtcagcctcatgacaccctgctattaagaaaaacccaacaatggagccctctgcagagatccccctcgggaaaggcaaggaaaccgggagaagccctctgatcgcaacaaggaggaagaccgggctggtcaaagccaagataaagaccaattcatccttctctatgttcaggagagcagatactatgcgaacccatgcacagttcaccaacattactgtggggtggaagtcgctttgtcctatcactgcagaaggacaggtcaatgtagccatataccatgagagcacccgagtccctgttctcaatctatggagtcctgtgtcctctagttggaagcacctggcaacaggcagtctaggctttgttagtctgaaccactgtccatatgtggttgaaggaagaatatcgggattcgagggagaagaagcagggttggtgacacttacacttcatcttgacactggattggagagtgatgacatccagaagtgtcgtttattttctgaacatccggagttagcgggtggatcactatatttatacacttcctacacaagtagccccataccggacagcaatctccctgccttaaggactgagatcgatgacactatcgctgcaatttggaggatgagtggagggaagatgtctatcaccacaagccatgctctagggctgattgccaccttcagggttaaaatagagtctttacttggggaatacccggatgatgcattctctgctcaggttgagaatcttgtgaaggattttctccttgtttcatcgacagctgagataaccagatcttacaggacctgccttgcaaaagtgctgtcgtcgatgaccacgtatgacaagaagtactgggacaccatcataacaggtccctcctccatatgacactcgccatcgcagcagctggagctcatattcccatcatcaagcacatctgcaagaataaaaatccgctcccacccttcattcatcctcacaaccaccctccgcccttgttaagaaaaacccaacatggatgctcctatcaatccttcctctcccacccggtcttcctatgagacagccctagagaggcaatggttgatcaaggatgaaccgtcacaccatgttgacttgggatgtcacttcacattagagatcaaagcattctcacccaaaatgattgagttgctggagactggtagcctgactctaggatccgtgtatcaagggctgtacaatttgatcaaggacaaagactgcaaggggctggagctagatcgggacaccacccatgggaacaaattgcatgtcttggcggcagctttcctgtgtgtgctcaggaagtatgagaaccaggtgcggttttacaatgtcaaaaccaaggactctctgacgggcgaaatccagtatatgtctagggtacacatcggagatgataacttggggctcttcgatccagacacctacaggatgataggagggtcatttccggaagggatctaccgactgaacctccgcacacaactcacacccaggggagaagatgacggtctggaaatcataatgggtaccattctaaccgtccccgtgcgcggtcctgctgaccgacagcctctcggagtattgatggacagacaactaagacagttcccggtagggaccaacccttggtcgtccttgggggaggcgaaacctaaagggagatcgttgtcactcctgcgtggaatccttatgtaattctgcatatcacctggttgatatccaataagaatgccactgcccagcattaccccacgattaagaaaaacccaacttacacagacgcaatcatgtctctcattcacctcatctacttccccaccatcctgtctctcatcctaggtacagagggcacaccagcattgacttatggcaacctggctgacaatcagcatggaaatgaggagcaaggggatgttgatatgtatcctctatatgagtgcagtaagaagggtgcaggagtctctagcaataactggtacggagcctgccgaggagcttgctctatgaccaaaaatactactgatgtccacatggagatatattacaggaatgacagtgtcggttggatagatgtattgtctatccagactacagaaatctggaagcactcacatgtaacctggtacggagagtgtgaacacaatacaaaatatgggagctcgaacacagcacctgtctccgtcattctggagcggttggagttccttcacaatgggatgtcatcatggccatacggtcatagcattcatgttcatgacactatctcaagccctgactgtagctattttggtgatgtgtccagatccggatggagattgataatatctaaacgtgctcttgaactgaaatcagatattgcaggagagggctatattgtagaccccgatgtggggtatgtatatcctgttgcggacggaattgggaaaggtaggacctggcatgtatggaaagctagctctgttccgtcttctggctgctacttcaaatcggcaggaattactaattgtaccctactaatggatacattcctctattcatgtccaagcctcaatatagccttctcagctcgggtcgggaaacacataaagagcacgtgcgtcggagatatgaatatatcaacagacggtgtgacttatcagactctcgggagcacggatcaagggtcgatatccacacagttggtatcattatggcatcaatcacaggaagctctcatacagcaacttattttgactatcaacgaggccctaggaaaaattgagacaagctactgtgagtccacttgtgatttgaccgagatgttaatctcccagcataccgagcacccgatggtaattgagacgcctgtcgggccatggctaccatacagcatagacggtaaagtttctgttctcccttgtcaaggtggtcaggatctggtggtgatcaagcctatagagatctgcgcttctccattcatgctcaaagttaaatcactcaagtctctagacacctactggtgggtccctacagatagccacgtggctccgttcacgcaatgcagcaacagggacgatgaagaaatgatcatcaagatacaacggaagaagccattgatatttgaattctggagaggagcctataagcttgattacccgtataactctagtggaatctggctggataaccctggaggtcatattcatagaagctctaaatggttccctacgttggatagtctatcctacagttctcctataaacttgccactgatcagcaaaggtataaggaaacatattcaagccatcgtcagtaccaccgagatcacatctggaggtaacgttacatcatggacccactcgatttcgactattacagaggccatgttcgactcagctgcgatagccgcgggtcgggtcgtgatgtggtggtacacactagaagaaagcgtcaaaaggtatctgactgtagcgttcggtctagtactaactgtcatcaccatttgggctatgtcaaaattattcttccgatcgtcgccaacatatgccagggcgcccacaaatatagaatgggtaaggtcatagacatcgcagtgtccacccacctcatctccatggagcccgatccacaataaaatgccatctatgtatcaaattaatcctatatgtatattaagaaaaacccaattccaatggatccagattatcctgatcttgatcccgagtctcttgatgttctcaacagcctacaagaagggtacgatgaggaggaagaagaggacagcggagtgtcactcaatggaatgggggactaccaccttaagtcagcactcaggtcgtacacagatatgatgaagcacccaatcttcaagaaggagttctccaaggcagtgattaactttggtatatctcacgactcgatgatgtcccagatagaaacaatgttctacatcctgaagagtactccattgtcaatgcaccttgggaccctttatggtgatctcttcactcgtcagagcacgctcggccacagcgaagatgtatttgacattatccaagcagagatacgaactcttcatcctcacctcctcctagagatgacacctgccaaggtcgaggagatgattctgattgctgacaggaagacatatgatgagagggcagcatctgcgttctggtccactctcataacaataaagaattacattccagcttggacagagaagggggcttgtactctggattggcctagtgtgtctctagacaagaagagtgggtacgtgaaagtgatggcaggtaaagatactaccatctacattgggactgatatttgtgtgatagagaagtatcctacagtaaaatgggctccactttcataccttttaaatggagctgacaaggtggcagaaaggctgaatgtgcgttactatagttgtctttgtgatcagcttgatataccagataggataacacttgatatgcttgatgaaatcatcaaagtgggggatgattgtcttcgagagatgggcaatgacggatacaatgtaattggatcctatgaggctttgcttgcaggaataatccaaaggagggacaaccagggactaataccggacagagatttgttgtggagaactactatggaagaatttacatccagccccggcaggaagtatctagaagagtggagcacaatgtttagttcattaagccctgaacagatagcctgtgctcatggactgtataggatctgggggcacccaatagttgatattcttggagggataaagaagatgcaagaggtggcatctgtgaagaagaatcctagacagagtgtactggatgaaataaggaggcaattcaaagaaatgttttttacctcatatcataggatacacaagcactaccctctacatacaatcttgaacagcctggataattcctatatactcaactgtctaaaggataacttgacgatcaacacatcagcaatctcctataacttccaggattgggactgtgtgatggtggagaaaaacttcgaagtgcctttctcttggaacctggttcacaatttgaaagataaagctatttccccgaacaggcaggagatttaccagacgatatcaactcgtggtacaatattcgggtctcagaaccgcagagggatactgaaatcactcacgatggagactgtccaactccgagactttcttcaaggcataaatgacaacgggttggatgacatagacaaaattattggggtctatcctaaagagcgtgagctgaagataaaggctagactgttctccctcatgtcattccggctcagattgtactgtgtgtcaacagaggcattattaggggacaaaatactcaagtatttcccgcaaatcacaatgtctcttgatatgctcacgatgataaagaagatgttcaaagtgtcctcccaaactactagagaggatgattcagtcacagtcatattcaatcttgacttcataaagtggaatctccagatgagacgtaatatatgtgagcctgtcttctcacaattaggtaaactctttggcatggagaatctgttcaataggactcatgagaccttccgagatagcctgatatacctctgttcgggggaaggagtactgtcagccgatccagtctacggagtattcccggacggtacatgggcatgggcgggtgatgagtcggggaaggaagggcttcgtcagaagggttggacaattctgactgtcgttacaatcatgttgattgcaaagcgacacaatgttgatgtatcactaatgggaggaggagacaatcaagtccttggtatcaccatatcaggagtgaccagagatcttcaaggagagctgattacagaatcggcctcactcgcccgaaggacaatcaagcaatttaccgaggacttattaaccactttctccgatctggggctccctttaaaagcatctgaaacatgggtcagtgactctctcttcatgtacaacaaacacatgttttacaagggaattcctctcagatcacctctgaaagccatctcaaggatcttcccgctggcaaacgacacaattatgaccatagacaacatgatcaataatatatcatcaggggtcaaagcagcctgtatgaaggagagacacggaattccacttgtattcatgaaaacactagcatataggaaggttgccgagggagcactgatcctacatccattgacgacttgcttcaggacaccaacattgcctgaaaatggaactgtgacgcgtgacggacggaagcgatcagtgaaggtgactaggaatcagctgagatcattctttacgttatgtatggtgggatcttccactatgggactacctggtactatacatctccctgatatgatcatgaggggattccctgatccattgaccagctacctgtcattcatttcagaattggttcccaagatcatggacccaagactagcgagcatcatcgcaacatcgggtcatatgagcatcaatagaatgacagagtatgcgaaacttgtggaggatcctgtatctataaatcacgacgcccccacacatggactgaatgagatccgacagttatctcgagacttcttgatgaacacgaccctggcagccaacacccatcttaagtcccttttcaccctcctagacagacagagagaaaaagaattctacgacgcactatgttctgctgaggaactggatgtgaaggtgctgcacgaagtagccggagcatcactattcggatatacaaacggcattgcctctcgtattgaccagacaagaacggtgagagcgttaaacgagacagtcaacgttatgaaaaggctagctgatgcagaggcaagatatattgcatatctgttcgctagggacatacataagcatgacctcatcccggatcagtgttccagagtgactgcggacaggtttgtttctgcttctacctttgatatatatataataattatcattaattagtagtaatataatatttcaaatatttttttcaaaataaaagaatgtagtatatagcaattgcttttctgtagtttataagtgtgtatattttaatttataacttttctaatatatgaccaaaacatggtgatgtttaggtatcgatattattcatggaagaagaaggttttaggggtaacagtacctcacccgattgagatgtgtcaagtcacatcggcgacccaaactgcattcgaggatgctgttatatgttggagtgataatctgagcggatcgagaatctacaccacaatgggtctagggaagatatatcaaggttcctacaccaaggaaaggttcaaggccacagacattgctgccgcgtatgggaacgaggacatcctgacaaaagcagtcaggctgcagaagctaattaactggagatatgatgaagggagcagctttgctaggataattcaacttgctctcagagccatcactgatgcaagcactgacggattccatagatctaaggaggagatcaaaggggaattcgatcatagaaggggcatatcaggagatatcagcggaggaattccaaatttccttgtcacacccacctcgcatttctcgtgtaccacatcctcttggatctcacatagcaggggagggaaaaacgagaatatccatttccagagtgtactgatcaacttattatacagagccatggtctatcgaggctcattgcccggtctaccagagatggtctggtattcaaaagagagatgcagccattgcattattgatattgtggaaccgtctccagaacgaacaaccccagctttgtcagagatgccatcagcacctaacaatccttttgcattcatagagtcagtaaatgtgaagttggattatcatcaccaggttgagatagaaaaaggaatggaagagaccaacctactaaattctgagtggggaggctcatctttaacaagtgtggatgaggctggactggtcttgtttctgatgctgattggatcccgtcaagtgtcagaatcgtttttgttattgatgagggagaagatagaacctcgaccgctactgcaagcagcattgaacagagtcatccttgtcaggaaactcggattggataggttgttccctataagaaacaccaaatgcatcaatcttatactggggtctgacagaggaatcactgcatgccatgacggctttggcctggaactgcacgggggatcctgggagaaagggatagagtgtgacacctcctttctttacaatgatactcacctcaagccctgtaaattgcatgttaaccttcatcttcaaaatgtgcctctccagctggccttggcgcatgcttcctataatcctgatgtttgtgagtgtcttgagtgcagagccatcattgaagatagagtctctaagaggatggtagggcaatataagagctggagatgccagtatcatgcggtcctgtccttctctccaatcatcatgagggtccacagtgagaggataatcaaaaatgagggagctatatatgaaggggatctgcatgtcccttatgtcgaggagatcattgctctggagaactcacaaaaattggcaatagatgtgacgtcatggagactgccgctgctgatgcacgcaacatgggagtctatcctgcctcagctgtatattactatgaaggcctgcctggtttgtcttcccttggagggtgtgattgtggatgatgacctggcactgatcaatctggtgtccaaggtgttggtcgatctgaggaggaccatccaggtctatatcagagcagggacgttctccgggtcggagatcaacaataagtatgacaacatcaggctgcttccagaggatcttagggacagcattcacatcctaacaaccacagacaaaccagatcctgctgcaactgtccaactggcagcggatgacacatcgcctcttgactgtgctgtgtaccatctcatctggggcgatacctggggcatgacgaggaaggtgcacggaagaattctcattccatcctctcgtgtgacaactggattcgcaggggtcatggtgacagatcactacgcaactgtggcagtattggagcttgctggtgctgttaacctgtgggagaaaaagctcattgatcttgaacatagaaacaacctcgatgttccgttgagcatgaatcgtgtgttggcaggttctagactcgggacccgggggataagatgggctcaatggacgtcgcctgcaggcctagagatcgttgtcaggaaagtgagggcaaagctgtcatcaatctcgggaaaccctggaaatgccgccaaatggaggaggaactgccagaaggtcctcaaggccttgatgatctccctgtatgctcattcgggcgaggacatgatgaggaatgcaagcggccttgtcaaggtgaacatacatgggaccctttcctctgtcaccgctgtgtgcgacccgtcggcggagagcagagtgcagagggcacagttcctgttcctaaaagaaaaggctcccaatgggcctttcattctaaggagcaggttggagaggcgcataaatctcctctcagttgtgtccgacctgctcgggtgatcaagaagtattaagaaaaaccctcatgttcacgtggttagaggggttacacccatactacatatcaaggtctttgacaatagtgtatttcgtgagaatgccatgtttttctgggtctctgggtcggcatggcatctccacctcctcgcggtccgacctgggcatccgaaggaggacgtccactcggatggctaagggagagctcgaattcggtacgctgaaatcaccagtctctctctacaaatctatctctctctattttctccataaataatgtgtgagtagtttcccgataagggaaattagggttcttatagggtttcgctcatgtgttgagcatataagaaacccttagtatgtatttgtatttgtaaaatacttctatcaataaaatttctaattcctaaaaccaaaatccagtactaaaatccagatccccaattcactggccgtcgttttacaacgtcgtgactgggaaaaccctggcgttacccaacttaatcgcctgtttaaactatcagtgtttgacagggacctgcagcccggccgggggatccactagcagattgtcgtttcccgccttcagtttaaactatcagtgtttgacaggatatattggcgggtaaacctaagagaaaagagcgtttattagaataatcggatatttaaaagggcgtgaaaaggtttatccgttcgtccatttgtatgtgcatgccaaccacaggagatctcagtaaagcgctggctgaacccccagccggaactgaccccacaaggccctagcgtttgcaatgcaccaggtcatcattgacccaggcgtgttccaccaggccgctgcctcgcaactcttcgcaggcttcgccgacctgctcgcgccacttcttcacgcgggtggaatccgatccgcacatgaggcggaaggtttccagcttgagcgggtacggctcccggtgcgagctgaaatagtcgaacatccgtcgggccgtcggcgacagcttgcggtacttctcccatatgaatttcgtgtagtggtcgccagcaaacagcacgacgatttcctcgtcgatcaggacctggcaacgggacgttttcttgccacggtccaggacgcggaagcggtgcagcagcgacaccgattccaggtgcccaacgcggtcggacgtgaagcccatcgccgtcgcctgtaggcgcgacaggcattcctcggccttcgtgtaataccggccattgatcgaccagcccaggtcctggcaaagctcgtagaacgtgaaggtgatcggctcgccgataggggtgcgcttcgcgtactccaacacctgctgccacaccagttcgtcatcgtcggcccgcagctcgacgccggtgtaggtgatcttcacgtccttgttgacgtggaaaatgaccttgttttgcagcgcctcgcgcgggattttcttgttgcgcgtggtgaacagggcagagcgggccgtgtcgtttggcatcgctcgcatcgtgtccggccacggcgcaatatcgaacaaggaaagctgcatttccttgatctgctgcttcgtgtgtttcagcaacgcggcctgcttggcctcgctgacctgttttgccaggtcctcgccggcggtttttcgcttcttggtcgtcatagttcctcgcgtgtcgatggtcatcgacttcgccaaacctgccgcctcctgttcgagacgacgcgaacgctccacggcggccgatggcgcgggcagggcagggggagccagttgcacgctgtcgcgctcgatcttggccgtagcttgctggaccatcgagccgacggactggaaggtttcgcggggcgcacgcatgacggtgcggcttgcgatggtttcggcatcctcggcggaaaaccccgcgtcgatcagttcttgcctgtatgccttccggtcaaacgtccgattcattcaccctccttgcgggattgccccgactcacgccggggcaatgtgcccttattcctgatttgacccgcctggtgccttggtgtccagataatccaccttatcggcaatgaagtcggtcccgtagaccgtctggccgtccttctcgtacttggtattccgaatcttgccctgcacgaataccagcgaccccttgcccaaatacttgccgtgggcctcggcctgagagccaaaacacttgatgcggaagaagtcggtgcgctcctgcttgtcgccggcatcgttgcgccacatctaggtactaaaacaattcatccagtaaaatataatattttattttctcccaatcaggcttgatccccagtaagtcaaaaaatagctcgacatactgttcttccccgatatcctccctgatcgaccggacgcagaaggcaatgtcataccacttgtccgccctgccgcttctcccaagatcaataaagccacttactttgccatctttcacaaagatgttgctgtctcccaggtcgccgtgggaaaagacaagttcctcttcgggcttttccgtctttaaaaaatcatacagctcgcgcggatctttaaatggagtgtcttcttcccagttttcgcaatccacatcggccagatcgttattcagtaagtaatccaattcggctaagcggctgtctaagctattcgtatagggacaatccgatatgtcgatggagtgaaagagcctgatgcactccgcatacagctcgataatcttttcagggctttgttcatcttcatactcttccgagcaaaggacgccatcggcctcactcatgagcagattgctccagccatcatgccgttcaaagtgcaggacctttggaacaggcagctttccttccagccatagcatcatgtccttttcccgttccacatcataggtggtccctttataccggctgtccgtcatttttaaatataggttttcattttctcccaccagcttatataccttagcaggagacattccttccgtatcttttacgcagcggtatttttcgatcagttttttcaattccggtgatattctcattttagccatttattatttccttcctcttttctacagtatttaaagataccccaagaagctaattataacaagacgaactccaattcactgttccttgcattctaaaaccttaaataccagaaaacagctttttcaaagttgttttcaaagttggcgtataacatagtatcgacggagccgattttgaaaccacaattatgggtgatgctgccaactcgagagcgggccgggagggttcgagaagggggggcaccccccttcggcgtgcgcggtcacgcgcacagggcgcagccctggttaaaaacaaggtttataaatattggtttaaaagcaggttaaaagacaggttagcggtggccgaaaaacgggcggaaacccttgcaaatgctggattttctgcctgtggacagcccctcaaatgtcaataggtgcgcccctcatctgtcagcactctgcccctcaagtgtcaaggatcgcgcccctcatctgtcagtagtcgcgcccctcaagtgtcaataccgcagggcacttatccccaggcttgtccacatcatctgtgggaaactcgcgtaaaatcaggcgttttcgccgatttgcgaggctggccagctccacgtcgccggccgaaatcgagcctgcccctcatctgtcaacgccgcgccgggtgagtcggcccctcaagtgtcaacgtccgcccctcatctgtcagtgagggccaagttttccgcgaggtatccacaacgccggcggccggccgcggtgtctcgcacacggcttcgacggcgtttctggcgcgtttgcagggccatagacggccgccagcccagcggcgagggcaaccagcccggtgagctctagtggactgatgggctgcctgtatcgagtggtgattttgtgccgagctgccggtcggggagctgttggctggct

SEQ ID NO: 10 - DNA, 22724 bp, Artificial sequence
  Synthetic nucleotide
ggtggcaggatatattgtggtgtaaacaaattgacgcttagacaacttaataacacattgcggacgtttttaatgtactggggtggttttggtaccgggccccccctcgaggtcgacggtatcgataagcttgatatcgaattcctgcaggcctcctaggactagttcccaggtcaacatggtggagcacgacactctcgtctactccaagaatatcaaagatacagtctcagaagaccagagggctattgagacttttcaacaaagggtaatatcgggaaacctcctcggattccattgcccagctatctgtcacttcatcgaaaggacagtagaaaaggaagatggcttctacaaatgccatcattgcgataaaggaaaggctatcgttcaagatgcctctaccgacagtggtcccaaagatgagacttttcaacaaagggtaatatcgggaaacctcctcggattccattgcccagctatctgtcacttcatcgaaaggacagtagaaaaggaagatggcttctacaaatgccatcattgcgataaaggaaaggctatcgttcaagatgcctctaccgacagtggtcccaaagatggacccccacccacgaggaacatcgtggaaaaagaagacgttccaaccacgtcttcaaagcaagtggattgatgtgatatctccactgacgtaagggatgacgcacaatcccactatccttcgcaagacccttcctctatataaggaagttcatttcatttggagaggagagacccatatatttcataaagggtcatctggtctgtatcacatagctgattagtcctcatgatgctgggcaagaatgcttgtgggaagatccgcccgcaacctgctcaataacacatgatcaggaagtgacacaatctgcgactattaacaaaaacccaaccacctctaaccttcttcatctcatccttcataatggcaaacatcaacatcccggatgacctggtatccaagtactcggaagatgtaaaggatctcacacaaaaggcaggtagcattccaagttctaaatcattgatcccccaaaccgcattcacccctgatgaattgaggagatctttgaagttctggcaagtgaccaccaaagtggcagcaactgtagcatccgactggactaatttggcgactgcattaaccaacgggacattcagtgcgacgcatctcaggacactgtgtgagctggcattcaacctcaaggccccaactggaacaggcacaatcttcattcatgagattgctgacccgtggaaagggtgcattgatgctaatgctcccgacactgtagcgatacctgcgactgacagtgatgttaatatgtctactgtcagctcaggttctaccgcctctgggacggaagatgcagaggtcaaaatgaaggcgatctcattcctatgctgcacattgctcaggctgtctgtgaaagagccgtcacacatcatgcaagcgatcaacagcatacgacagaggttcggctccctgtacggtgtctcctcccctaccttgaatagtgttacattcaacaggagccaactttctcgcatcaagcaaggcattgaaacatatccctctgctcggggtacagtattctattacaccaggtatgccgatgtcacgcatgggtatactaccaaagagtatgggatatgcaggttcctactgttccaacaccttgaacttgaggggatgcacatctataagatgctcttagctctacttggcgagtggtcaacagttccaattgggctcttgctcacctggatcaggaacccgaggtctaagttggccataaatgagattgtcaagatagtcaaggagcttgataagccggaagtggacaagggctggaaatatgctcggatggtcaataacacattcttccttgatctatcctctagaaggaacacttacctatgtgctgtgttggcctccctgaacaagaagaacgttccgcaaggatcaggggaatatgctgaccccactaacatagctgtgatcaagaatatggaccaatctgtgaaatcacaggttgccacagatgtgaccctgattgagagaatttatgagaaatatctggtgtctgcaggtggagacgaggcaggaacagcatatgctctgtctcgcggagttaagagatcctcgcctcaaagtcaggaaggcggtcaatccggccagtctggaggaacacccatggaagtcgacggagcatctggtagaggagcagcaggtccggccccgaagaagacacgatcaggcttatagatctcagctcggtatcaatagcctacatgagaagatgtaccacgtgccatacgctcctgaacctatgtgtgatcccaatggaccaacaataatagcagagtctgtcataaccctgccgttttaatcaattatcatgtgcttcccatgttgttgtttcaatctattaagaaaaacccaacctcaggttcagagcaccagtggtctagtggtagaatagtaccctgccacggtacagacccgggttcgattcccggctggtgcaccgttcgtcgtgatagatgggttttagagctagaaatagcaagttaaaataaggctagtccgttatcaacttgaaaaagtggcaccgagtcggtgcttcagagcaccagtggtctagtggtagaatagtaccctgccacggtacagacccgggttcgattcccggctggtgcaatctcagctcggtatcaatagcctacatgagaagatgtaccacgtgccatacgctcctgaacctatgtgtgatcccaatggaccaacaataatagcagagtctgtcataaccctgccgttttaatcaattatcatgtgcttcccatgttgttgtttcaatctattaagaaaaacccaacctcaggatggactataaggatcacgatggcgactacaaggatcatgacattgactataaggatgacgacgataagatggcacctaagaagaaaaggaaagtcggcattcatggcgttccggcagccgacaaaaagtatagcatcggcctcgatattgggacaaactctgtgggctgggcggtaattaccgacgagtacaaggtgcctagtaagaaatttaaagtgctcggaaacactgacaggcactctataaagaagaacctgatcggggcactgcttttcgactccggagagacggcggaggcgacgcgtctcaagcgtaccgcgcgccgcaggtacacaagaaggaagaataggatctgctacttgcaggaaatcttcagtaacgagatggcgaaggtcgacgatagtttctttcatcggttggaagaatcgttcctcgtagaggaggacaaaaagcacgagcgtcacccaatattcgggaatattgttgacgaggttgcctaccatgagaaatatcctacaatatatcacctccgtaagaagcttgtcgattcaactgataaggctgatctcagactcatctatcttgccctcgcacatatgattaagtttcgtggccacttcttgattgaaggcgacctcaacccggacaactcagatgttgacaagctttttatacagctcgtccagacatataaccagctgtttgaagagaatcccatcaatgcgagtggggttgatgctaaggccattttgtccgccaggttgtccaaatctcgcagactggaaaacctgatcgcacagcttcccggtgaaaagaaaaacgggctcttcggcaatctcatcgcactgtccctcggcctcaccccaaacttcaagtctaacttcgacctggccgaggatgcgaagctccagctgtcaaaagatacatacgacgacgatttggacaatctgcttgcgcaaataggcgaccagtatgcggacctgttcctggctgccaaaaatctgtcagatgcaatcctcctgtccgatatattgcgtgtgaacaccgaaatcacgaaggcaccgcttagcgcatccatgatcaagagatacgacgagcaccatcaggacctcacactcctcaaggcgcttgttcgtcagcagcttcccgagaaatataaggaaatttttttcgatcaaagcaagaatggatatgctggctatattgacggtggcgcttcgcaggaggagttctataaattcattaagccgattctggagaagatggacggaacggaggagctcctcgtcaagcttaaccgggaagacctgttgcggaagcagaggacttttgataacggctctattccgcaccaaatccatctgggtgagttgcacgcaatcttgagaagacaagaggatttctacccgttccttaaggataacagagagaagatagaaaaaatactgaccttcaggataccatactatgtgggcccactggcgcgcggaaatagtcgtttcgcatggatgactagaaagtccgaagaaacgatcacgccatggaattttgaggaagtggtcgacaagggcgcctctgcccagagcttcatcgaaaggatgaccaattttgacaaaaatctgcctaacgaaaaggtgcttccgaagcacagcctgttgtatgaatacttcacagtttataacgagctcactaaggtcaagtacgtcacggagggcatgcgtaagcctgctttcctgtctggtgaacaaaaaaaggcgattgtggacctccttttcaagacgaaccgtaaagttactgtgaagcaactgaaagaggattactttaagaaaattgagtgcttcgacagtgtggagatttccggtgtcgaggaccggtttaacgccagcctgggtacgtatcatgacctgcttaaaattatcaaggataaagatttcctggataatgaagagaacgaagatatactggaggacattgtgttgactttgaccctcttcgaggacagagagatgattgaggaaagactgaagacctacgcacacctttttgatgacaaggtcatgaaacaactcaagcgccggcgctatactggctggggccggctttctcgcaagctcatcaatgggattcgggataagcaatcaggcaagacaattttggacttcctcaaatccgacggattcgcaaataggaattttatgcagctgatacatgacgactctttgacattcaaagaagacatacagaaggctcaggtctccggccaaggagattctttgcacgagcatatcgctaacttggcaggtagccccgccataaaaaagggcattcttcaaacggtaaaagttgttgacgaactcgtgaaggttatgggccgtcataagccggaaaacattgttattgaaatggctagggaaaatcagacgacccagaagggacagaaaaatagcagggagcggatgaagagaattgaagagggaattaaggagcttggatctcagattcttaaggagcaccctgtggagaacacccaacttcagaatgaaaagctctacctttactaccttcaaaacggccgggatatgtacgtcgatcaggaacttgacattaaccggttgagcgattatgacgttgaccatattgtgccccaatctttccttaaagacgactctatcgacaataaagtgctgacgcgcagcgataaaaatcgcggtaagtcggataatgtcccgtcggaagaggtggttaaaaaaatgaagaactattggaggcaactcctgaatgccaagctgatcactcagaggaaattcgacaatctcaccaaggcagaaaggggtggacttagcgagctcgacaaggccggttttatcaaaagacagctggtggagacacgccaaatcaccaaacacgttgcccagatcctggattcgaggatgaacacgaagtatgacgagaacgacaagttgattagggaagtcaaggtcatcactttgaagtccaagctggtgagcgactttcgcaaagacttccagttttacaaagtcagggaaattaataactaccaccacgcccacgacgcctaccttaacgccgtggttggcacagcactcatcaagaaataccctaagctcgaatctgagttcgtctatggcgactataaggtctacgacgttagaaaaatgatcgcgaaatctgagcaggaaataggcaaggcaactgccaagtacttcttctattccaatatcatgaacttttttaagacggagattaccctggcgaatggtgagatccgcaagcgccctttgattgagacaaacggagaaacaggagagatcgtatgggacaaagggcgggactttgctactgttaggaaggtgctctctatgccacaagttaacattgtcaaaaaaactgaagtgcagacaggtgggtttagcaaggaatctatcctgccgaagaggaactctgacaagctgatcgcccgcaagaaagattgggatccgaaaaagtacggaggattcgactcccccacagttgcgtactccgtgcttgtcgtggccaaagtggagaagggcaagtctaagaagctcaagagcgtcaaagagttgttggggatcacgattatggagcggtcgtctttcgaaaagaatccgatagattttctcgaggccaagggttataaagaagtcaagaaggatcttatcatcaagctccctaagtactccctctttgagcttgaaaacggacggaaaagaatgctggcttcagcgggtgaacttcagaagggtaatgaactcgctctgccctcaaaatatgtgaatttcctttacctggcatcacactatgagaagcttaaggggtctccagaggacaacgagcagaagcaactgttcgttgaacaacacaagcactaccttgacgagattatcgagcaaatcagcgagtttagcaagcgcgttatactggcagacgcaaatcttgataaggtccttagcgcctacaacaagcatagagacaaacccatccgggagcaggccgagaacattattcatctcttcaccttgacgaatcttggggccccggccgcgttcaagtacttcgatactaccatagacagaaagcgctatacatcgacaaaggaagttcttgacgccacgctgatccaccaaagtataacaggcctctatgagacacgcatcgacctttcgcagttgggcggtgaccgccccaaaaagaagaggaaagttggcgggtgaatctcagctcggtatcaatagcctacatgagaagatgtaccacgtgccatacgctcctgaacctatgtgtgatcccaatggaccaacaataatagcagagtctgtcataaccctgccgttttaatcaattatcatgtgcttcccatgttgttgtttcaatctattaagaaaaacccaacctcaggatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaaatctcagctcggtatcaatagcctacatgagaagatgtaccacgtgccatacgctcctgaacctatgtgtgatcccaatggaccaacaataatagcagagtctgtcataaccctgccgttttaatcaattatcatgtgcttcccatgttgttgtttcaatctattaagaaaaacccaacctcaggatgaatcgttactcccgtcgttctagacatcccaaccctcctgtccccaatcaggaagaacccgagcgcgacccgaatcacatagatcaagacctcgcagacttggctcaacccttggtcctaaaagaaagacacgcagtaatggcaccaactcagcccagccttagcgatgtgatcaatgaggaaagacaggctcccatcacgtttggtaatcctccagaggtcatggcgaacgctaggctatcagcgctgggttatgacaatctcacggagagggagaagaggatccttgctgtaggaatcagatgcggagaggctgccaaagactatcattcgctcactaccaccaaaaagtggattgaggatgagctgaagtctcaaatggtggctctcgcatcatcaacaaggactctcactgaggctgcatcacttcacacgacctttgcaatgctgcattctccatccattaaaagaaaggcagaagcaatgtctcacatatctcaaggcgaagagagcattgacatttctaagctgaataagactggaatggaggacatctgggtggcaatggagagtgagtcaaaagaggatgcagtagacacgtatctgagaaacattctggaagtggacccgacacaattctatgcaatagacggctggggcctctacctggacttcattcccacttggcattatatagctgcagggaagaattcggcgcaatttaagacgagttatgctgacgagattgtagagcagcgtgctgcattcgagagggtcctctctaagcgccctagggttgagatctaagcctgtatgtcactccactccccaataatgtccacgtcagcctcatgacaccctgctattaagaaaaacccaacaatggagccctctgcagagatccccctcgggaaaggcaaggaaaccgggagaagccctctgatcgcaacaaggaggaagaccgggctggtcaaagccaagataaagaccaattcatccttctctatgttcaggagagcagatactatgcgaacccatgcacagttcaccaacattactgtggggtggaagtcgctttgtcctatcactgcagaaggacaggtcaatgtagccatataccatgagagcacccgagtccctgttctcaatctatggagtcctgtgtcctctagttggaagcacctggcaacaggcagtctaggctttgttagtctgaaccactgtccatatgtggttgaaggaagaatatcgggattcgagggagaagaagcagggttggtgacacttacacttcatcttgacactggattggagagtgatgacatccagaagtgtcgtttattttctgaacatccggagttagcgggtggatcactatatttatacacttcctacacaagtagccccataccggacagcaatctccctgccttaaggactgagatcgatgacactatcgctgcaatttggaggatgagtggagggaagatgtctatcaccacaagccatgctctagggctgattgccaccttcagggttaaaatagagtctttacttggggaatacccggatgatgcattctctgctcaggttgagaatcttgtgaaggattttctccttgtttcatcgacagctgagataaccagatcttacaggacctgccttgcaaaagtgctgtcgtcgatgaccacgtatgacaagaagtactgggacaccatcataacaggtccctcctccatatgacactcgccatcgcagcagctggagctcatattcccatcatcaagcacatctgcaagaataaaaatccgctcccacccttcattcatcctcacaaccaccctccgcccttgttaagaaaaacccaacatggatgctcctatcaatccttcctctcccacccggtcttcctatgagacagccctagagaggcaatggttgatcaaggatgaaccgtcacaccatgttgacttgggatgtcacttcacattagagatcaaagcattctcacccaaaatgattgagttgctggagactggtagcctgactctaggatccgtgtatcaagggctgtacaatttgatcaaggacaaagactgcaaggggctggagctagatcgggacaccacccatgggaacaaattgcatgtcttggcggcagctttcctgtgtgtgctcaggaagtatgagaaccaggtgcggttttacaatgtcaaaaccaaggactctctgacgggcgaaatccagtatatgtctagggtacacatcggagatgataacttggggctcttcgatccagacacctacaggatgataggagggtcatttccggaagggatctaccgactgaacctccgcacacaactcacacccaggggagaagatgacggtctggaaatcataatgggtaccattctaaccgtccccgtgcgcggtcctgctgaccgacagcctctcggagtattgatggacagacaactaagacagttcccggtagggaccaacccttggtcgtccttgggggaggcgaaacctaaagggagatcgttgtcactcctgcgtggaatccttatgtaattctgcatatcacctggttgatatccaataagaatgccactgcccagcattaccccacgattaagaaaaacccaacttacacagacgcaatcatgtctctcattcacctcatctacttccccaccatcctgtctctcatcctaggtacagagggcacaccagcattgacttatggcaacctggctgacaatcagcatggaaatgaggagcaaggggatgttgatatgtatcctctatatgagtgcagtaagaagggtgcaggagtctctagcaataactggtacggagcctgccgaggagcttgctctatgaccaaaaatactactgatgtccacatggagatatattacaggaatgacagtgtcggttggatagatgtattgtctatccagactacagaaatctggaagcactcacatgtaacctggtacggagagtgtgaacacaatacaaaatatgggagctcgaacacagcacctgtctccgtcattctggagcggttggagttccttcacaatgggatgtcatcatggccatacggtcatagcattcatgttcatgacactatctcaagccctgactgtagctattttggtgatgtgtccagatccggatggagattgataatatctaaacgtgctcttgaactgaaatcagatattgcaggagagggctatattgtagaccccgatgtggggtatgtatatcctgttgcggacggaattgggaaaggtaggacctggcatgtatggaaagctagctctgttccgtcttctggctgctacttcaaatcggcaggaattactaattgtaccctactaatggatacattcctctattcatgtccaagcctcaatatagccttctcagctcgggtcgggaaacacataaagagcacgtgcgtcggagatatgaatatatcaacagacggtgtgacttatcagactctcgggagcacggatcaagggtcgatatccacacagttggtatcattatggcatcaatcacaggaagctctcatacagcaacttattttgactatcaacgaggccctaggaaaaattgagacaagctactgtgagtccacttgtgatttgaccgagatgttaatctcccagcataccgagcacccgatggtaattgagacgcctgtcgggccatggctaccatacagcatagacggtaaagtttctgttctcccttgtcaaggtggtcaggatctggtggtgatcaagcctatagagatctgcgcttctccattcatgctcaaagttaaatcactcaagtctctagacacctactggtgggtccctacagatagccacgtggctccgttcacgcaatgcagcaacagggacgatgaagaaatgatcatcaagatacaacggaagaagccattgatatttgaattctggagaggagcctataagcttgattacccgtataactctagtggaatctggctggataaccctggaggtcatattcatagaagctctaaatggttccctacgttggatagtctatcctacagttctcctataaacttgccactgatcagcaaaggtataaggaaacatattcaagccatcgtcagtaccaccgagatcacatctggaggtaacgttacatcatggacccactcgatttcgactattacagaggccatgttcgactcagctgcgatagccgcgggtcgggtcgtgatgtggtggtacacactagaagaaagcgtcaaaaggtatctgactgtagcgttcggtctagtactaactgtcatcaccatttgggctatgtcaaaattattcttccgatcgtcgccaacatatgccagggcgcccacaaatatagaatgggtaaggtcatagacatcgcagtgtccacccacctcatctccatggagcccgatccacaataaaatgccatctatgtatcaaattaatcctatatgtatattaagaaaaacccaattccaatggatccagattatcctgatcttgatcccgagtctcttgatgttctcaacagcctacaagaagggtacgatgaggaggaagaagaggacagcggagtgtcactcaatggaatgggggactaccaccttaagtcagcactcaggtcgtacacagatatgatgaagcacccaatcttcaagaaggagttctccaaggcagtgattaactttggtatatctcacgactcgatgatgtcccagatagaaacaatgttctacatcctgaagagtactccattgtcaatgcaccttgggaccctttatggtgatctcttcactcgtcagagcacgctcggccacagcgaagatgtatttgacattatccaagcagagatacgaactcttcatcctcacctcctcctagagatgacacctgccaaggtcgaggagatgattctgattgctgacaggaagacatatgatgagagggcagcatctgcgttctggtccactctcataacaataaagaattacattccagcttggacagagaagggggcttgtactctggattggcctagtgtgtctctagacaagaagagtgggtacgtgaaagtgatggcaggtaaagatactaccatctacattgggactgatatttgtgtgatagagaagtatcctacagtaaaatgggctccactttcataccttttaaatggagctgacaaggtggcagaaaggctgaatgtgcgttactatagttgtctttgtgatcagcttgatataccagataggataacacttgatatgcttgatgaaatcatcaaagtgggggatgattgtcttcgagagatgggcaatgacggatacaatgtaattggatcctatgaggctttgcttgcaggaataatccaaaggagggacaaccagggactaataccggacagagatttgttgtggagaactactatggaagaatttacatccagccccggcaggaagtatctagaagagtggagcacaatgtttagttcattaagccctgaacagatagcctgtgctcatggactgtataggatctgggggcacccaatagttgatattcttggagggataaagaagatgcaagaggtggcatctgtgaagaagaatcctagacagagtgtactggatgaaataaggaggcaattcaaagaaatgttttttacctcatatcataggatacacaagcactaccctctacatacaatcttgaacagcctggataattcctatatactcaactgtctaaaggataacttgacgatcaacacatcagcaatctcctataacttccaggattgggactgtgtgatggtggagaaaaacttcgaagtgcctttctcttggaacctggttcacaatttgaaagataaagctatttccccgaacaggcaggagatttaccagacgatatcaactcgtggtacaatattcgggtctcagaaccgcagagggatactgaaatcactcacgatggagactgtccaactccgagactttcttcaaggcataaatgacaacgggttggatgacatagacaaaattattggggtctatcctaaagagcgtgagctgaagataaaggctagactgttctccctcatgtcattccggctcagattgtactgtgtgtcaacagaggcattattaggggacaaaatactcaagtatttcccgcaaatcacaatgtctcttgatatgctcacgatgataaagaagatgttcaaagtgtcctcccaaactactagagaggatgattcagtcacagtcatattcaatcttgacttcataaagtggaatctccagatgagacgtaatatatgtgagcctgtcttctcacaattaggtaaactctttggcatggagaatctgttcaataggactcatgagaccttccgagatagcctgatatacctctgttcgggggaaggagtactgtcagccgatccagtctacggagtattcccggacggtacatgggcatgggcgggtgatgagtcggggaaggaagggcttcgtcagaagggttggacaattctgactgtcgttacaatcatgttgattgcaaagcgacacaatgttgatgtatcactaatgggaggaggagacaatcaagtccttggtatcaccatatcaggagtgaccagagatcttcaaggagagctgattacagaatcggcctcactcgcccgaaggacaatcaagcaatttaccgaggacttattaaccactttctccgatctggggctccctttaaaagcatctgaaacatgggtcagtgactctctcttcatgtacaacaaacacatgttttacaagggaattcctctcagatcacctctgaaagccatctcaaggatcttcccgctggcaaacgacacaattatgaccatagacaacatgatcaataatatatcatcaggggtcaaagcagcctgtatgaaggagagacacggaattccacttgtattcatgaaaacactagcatataggaaggttgccgagggagcactgatcctacatccattgacgacttgcttcaggacaccaacattgcctgaaaatggaactgtgacgcgtgacggacggaagcgatcagtgaaggtgactaggaatcagctgagatcattctttacgttatgtatggtgggatcttccactatgggactacctggtactatacatctccctgatatgatcatgaggggattccctgatccattgaccagctacctgtcattcatttcagaattggttcccaagatcatggacccaagactagcgagcatcatcgcaacatcgggtcatatgagcatcaatagaatgacagagtatgcgaaacttgtggaggatcctgtatctataaatcacgacgcccccacacatggactgaatgagatccgacagttatctcgagacttcttgatgaacacgaccctggcagccaacacccatcttaagtcccttttcaccctcctagacagacagagagaaaaagaattctacgacgcactatgttctgctgaggaactggatgtgaaggtgctgcacgaagtagccggagcatcactattcggatatacaaacggcattgcctctcgtattgaccagacaagaacggtgagagcgttaaacgagacagtcaacgttatgaaaaggctagctgatgcagaggcaagatatattgcatatctgttcgctagggacatacataagcatgacctcatcccggatcagtgttccagagtgactgcggacaggtttgtttctgcttctacctttgatatatatataataattatcattaattagtagtaatataatatttcaaatatttttttcaaaataaaagaatgtagtatatagcaattgcttttctgtagtttataagtgtgtatattttaatttataacttttctaatatatgaccaaaacatggtgatgtttaggtatcgatattattcatggaagaagaaggttttaggggtaacagtacctcacccgattgagatgtgtcaagtcacatcggcgacccaaactgcattcgaggatgctgttatatgttggagtgataatctgagcggatcgagaatctacaccacaatgggtctagggaagatatatcaaggttcctacaccaaggaaaggttcaaggccacagacattgctgccgcgtatgggaacgaggacatcctgacaaaagcagtcaggctgcagaagctaattaactggagatatgatgaagggagcagctttgctaggataattcaacttgctctcagagccatcactgatgcaagcactgacggattccatagatctaaggaggagatcaaaggggaattcgatcatagaaggggcatatcaggagatatcagcggaggaattccaaatttccttgtcacacccacctcgcatttctcgtgtaccacatcctcttggatctcacatagcaggggagggaaaaacgagaatatccatttccagagtgtactgatcaacttattatacagagccatggtctatcgaggctcattgcccggtctaccagagatggtctggtattcaaaagagagatgcagccattgcattattgatattgtggaaccgtctccagaacgaacaaccccagctttgtcagagatgccatcagcacctaacaatccttttgcattcatagagtcagtaaatgtgaagttggattatcatcaccaggttgagatagaaaaaggaatggaagagaccaacctactaaattctgagtggggaggctcatctttaacaagtgtggatgaggctggactggtcttgtttctgatgctgattggatcccgtcaagtgtcagaatcgtttttgttattgatgagggagaagatagaacctcgaccgctactgcaagcagcattgaacagagtcatccttgtcaggaaactcggattggataggttgttccctataagaaacaccaaatgcatcaatcttatactggggtctgacagaggaatcactgcatgccatgacggctttggcctggaactgcacgggggatcctgggagaaagggatagagtgtgacacctcctttctttacaatgatactcacctcaagccctgtaaattgcatgttaaccttcatcttcaaaatgtgcctctccagctggccttggcgcatgcttcctataatcctgatgtttgtgagtgtcttgagtgcagagccatcattgaagatagagtctctaagaggatggtagggcaatataagagctggagatgccagtatcatgcggtcctgtccttctctccaatcatcatgagggtccacagtgagaggataatcaaaaatgagggagctatatatgaaggggatctgcatgtcccttatgtcgaggagatcattgctctggagaactcacaaaaattggcaatagatgtgacgtcatggagactgccgctgctgatgcacgcaacatgggagtctatcctgcctcagctgtatattactatgaaggcctgcctggtttgtcttcccttggagggtgtgattgtggatgatgacctggcactgatcaatctggtgtccaaggtgttggtcgatctgaggaggaccatccaggtctatatcagagcagggacgttctccgggtcggagatcaacaataagtatgacaacatcaggctgcttccagaggatcttagggacagcattcacatcctaacaaccacagacaaaccagatcctgctgcaactgtccaactggcagcggatgacacatcgcctcttgactgtgctgtgtaccatctcatctggggcgatacctggggcatgacgaggaaggtgcacggaagaattctcattccatcctctcgtgtgacaactggattcgcaggggtcatggtgacagatcactacgcaactgtggcagtattggagcttgctggtgctgttaacctgtgggagaaaaagctcattgatcttgaacatagaaacaacctcgatgttccgttgagcatgaatcgtgtgttggcaggttctagactcgggacccgggggataagatgggctcaatggacgtcgcctgcaggcctagagatcgttgtcaggaaagtgagggcaaagctgtcatcaatctcgggaaaccctggaaatgccgccaaatggaggaggaactgccagaaggtcctcaaggccttgatgatctccctgtatgctcattcgggcgaggacatgatgaggaatgcaagcggccttgtcaaggtgaacatacatgggaccctttcctctgtcaccgctgtgtgcgacccgtcggcggagagcagagtgcagagggcacagttcctgttcctaaaagaaaaggctcccaatgggcctttcattctaaggagcaggttggagaggcgcataaatctcctctcagttgtgtccgacctgctcgggtgatcaagaagtattaagaaaaaccctcatgttcacgtggttagaggggttacacccatactacatatcaaggtctttgacaatagtgtatttcgtgagaatgccatgtttttctgggtctctgggtcggcatggcatctccacctcctcgcggtccgacctgggcatccgaaggaggacgtccactcggatggctaagggagagctcgaattcggtacgctgaaatcaccagtctctctctacaaatctatctctctctattttctccataaataatgtgtgagtagtttcccgataagggaaattagggttcttatagggtttcgctcatgtgttgagcatataagaaacccttagtatgtatttgtatttgtaaaatacttctatcaataaaatttctaattcctaaaaccaaaatccagtactaaaatccagatccccaattcactggccgtcgttttacaacgtcgtgactgggaaaaccctggcgttacccaacttaatcgcctgtttaaactatcagtgtttgacagggacctgcagcccggccgggggatccactagcagattgtcgtttcccgccttcagtttaaactatcagtgtttgacaggatatattggcgggtaaacctaagagaaaagagcgtttattagaataatcggatatttaaaagggcgtgaaaaggtttatccgttcgtccatttgtatgtgcatgccaaccacaggagatctcagtaaagcgctggctgaacccccagccggaactgaccccacaaggccctagcgtttgcaatgcaccaggtcatcattgacccaggcgtgttccaccaggccgctgcctcgcaactcttcgcaggcttcgccgacctgctcgcgccacttcttcacgcgggtggaatccgatccgcacatgaggcggaaggtttccagcttgagcgggtacggctcccggtgcgagctgaaatagtcgaacatccgtcgggccgtcggcgacagcttgcggtacttctcccatatgaatttcgtgtagtggtcgccagcaaacagcacgacgatttcctcgtcgatcaggacctggcaacgggacgttttcttgccacggtccaggacgcggaagcggtgcagcagcgacaccgattccaggtgcccaacgcggtcggacgtgaagcccatcgccgtcgcctgtaggcgcgacaggcattcctcggccttcgtgtaataccggccattgatcgaccagcccaggtcctggcaaagctcgtagaacgtgaaggtgatcggctcgccgataggggtgcgcttcgcgtactccaacacctgctgccacaccagttcgtcatcgtcggcccgcagctcgacgccggtgtaggtgatcttcacgtccttgttgacgtggaaaatgaccttgttttgcagcgcctcgcgcgggattttcttgttgcgcgtggtgaacagggcagagcgggccgtgtcgtttggcatcgctcgcatcgtgtccggccacggcgcaatatcgaacaaggaaagctgcatttccttgatctgctgcttcgtgtgtttcagcaacgcggcctgcttggcctcgctgacctgttttgccaggtcctcgccggcggtttttcgcttcttggtcgtcatagttcctcgcgtgtcgatggtcatcgacttcgccaaacctgccgcctcctgttcgagacgacgcgaacgctccacggcggccgatggcgcgggcagggcagggggagccagttgcacgctgtcgcgctcgatcttggccgtagcttgctggaccatcgagccgacggactggaaggtttcgcggggcgcacgcatgacggtgcggcttgcgatggtttcggcatcctcggcggaaaaccccgcgtcgatcagttcttgcctgtatgccttccggtcaaacgtccgattcattcaccctccttgcgggattgccccgactcacgccggggcaatgtgcccttattcctgatttgacccgcctggtgccttggtgtccagataatccaccttatcggcaatgaagtcggtcccgtagaccgtctggccgtccttctcgtacttggtattccgaatcttgccctgcacgaataccagcgaccccttgcccaaatacttgccgtgggcctcggcctgagagccaaaacacttgatgcggaagaagtcggtgcgctcctgcttgtcgccggcatcgttgcgccacatctaggtactaaaacaattcatccagtaaaatataatattttattttctcccaatcaggcttgatccccagtaagtcaaaaaatagctcgacatactgttcttccccgatatcctccctgatcgaccggacgcagaaggcaatgtcataccacttgtccgccctgccgcttctcccaagatcaataaagccacttactttgccatctttcacaaagatgttgctgtctcccaggtcgccgtgggaaaagacaagttcctcttcgggcttttccgtctttaaaaaatcatacagctcgcgcggatctttaaatggagtgtcttcttcccagttttcgcaatccacatcggccagatcgttattcagtaagtaatccaattcggctaagcggctgtctaagctattcgtatagggacaatccgatatgtcgatggagtgaaagagcctgatgcactccgcatacagctcgataatcttttcagggctttgttcatcttcatactcttccgagcaaaggacgccatcggcctcactcatgagcagattgctccagccatcatgccgttcaaagtgcaggacctttggaacaggcagctttccttccagccatagcatcatgtccttttcccgttccacatcataggtggtccctttataccggctgtccgtcatttttaaatataggttttcattttctcccaccagcttatataccttagcaggagacattccttccgtatcttttacgcagcggtatttttcgatcagttttttcaattccggtgatattctcattttagccatttattatttccttcctcttttctacagtatttaaagataccccaagaagctaattataacaagacgaactccaattcactgttccttgcattctaaaaccttaaataccagaaaacagctttttcaaagttgttttcaaagttggcgtataacatagtatcgacggagccgattttgaaaccacaattatgggtgatgctgccaactcgagagcgggccgggagggttcgagaagggggggcaccccccttcggcgtgcgcggtcacgcgcacagggcgcagccctggttaaaaacaaggtttataaatattggtttaaaagcaggttaaaagacaggttagcggtggccgaaaaacgggcggaaacccttgcaaatgctggattttctgcctgtggacagcccctcaaatgtcaataggtgcgcccctcatctgtcagcactctgcccctcaagtgtcaaggatcgcgcccctcatctgtcagtagtcgcgcccctcaagtgtcaataccgcagggcacttatccccaggcttgtccacatcatctgtgggaaactcgcgtaaaatcaggcgttttcgccgatttgcgaggctggccagctccacgtcgccggccgaaatcgagcctgcccctcatctgtcaacgccgcgccgggtgagtcggcccctcaagtgtcaacgtccgcccctcatctgtcagtgagggccaagttttccgcgaggtatccacaacgccggcggccggccgcggtgtctcgcacacggcttcgacggcgtttctggcgcgtttgcagggccatagacggccgccagcccagcggcgagggcaaccagcccggtgagctctagtggactgatgggctgcctgtatcgagtggtgattttgtgccgagctgccggtcggggagctgttggctggct

SEQ ID NO: 11 - DNA, 20 bp, Artificial sequence
  Synthetic nucleotide
ctgcggacaggtatcgatat

SEQ ID NO: 12 - DNA, 20 bp, Artificial sequence
  Synthetic nucleotide
ctgcggacaggtatcgatat

SEQ ID NO: 13 - DNA, 18 bp, Artificial sequence
  Synthetic nucleotide
gggtcggcatggcatctc

SEQ ID NO: 14 - DNA, 24 bp, Artificial sequence
  Synthetic nucleotide
cctctccaaatgaaatgaacttcc

SEQ ID NO: 15 - DNA, 39 bp, Artificial sequence
  Synthetic nucleotide
catttcatttggagaggagagacccatatatttcataaa

SEQ ID NO: 16 - DNA, 39 bp, Artificial sequence
  Synthetic nucleotide
gagatgccatgccgacccagagacccagaaaaacatggc

SEQ ID NO: 17 - DNA, 19 bp, Artificial sequence
  Synthetic nucleotide
atggcaaacatcaacatcc

SEQ ID NO: 18 - DNA, 22 bp, Artificial sequence
  Synthetic nucleotide
ctataagcctgatcgtgtcttc

SEQ ID NO: 19 - DNA, 20 bp, Artificial sequence
  Synthetic nucleotide
atgaatcgttactcccgtcg

SEQ ID NO: 20 - DNA, 20 bp, Artificial sequence
  Synthetic nucleotide
ttagatctcaaccctagggc

SEQ ID NO: 21 - DNA, 39 bp, Artificial sequence
  Synthetic nucleotide
catttcatttggagaggatggatccagattatcctgatc

SEQ ID NO: 22 - DNA, 23 bp, Artificial sequence
  Synthetic nucleotide
aattcctaaaaccaaaatccagt

SEQ ID NO: 23 - DNA, 39 bp, Artificial sequence
  Synthetic nucleotide
gagatgccatgccgaccctcacccgagcaggtcggacac

SEQ ID NO: 24 - DNA, 34 bp, Artificial sequence
  Synthetic nucleotide
gtcggatctcattcagtccatgtgtgggggcgtc

SEQ ID NO: 25 - DNA, 34 bp, Artificial sequence
  Synthetic nucleotide
gacgcccccacacatggactgaatgagatccgac

SEQ ID NO: 26 - DNA, 23 bp, Artificial sequence
  Synthetic nucleotide
tgtctgcaggtggagacgaggca

SEQ ID NO: 27 - DNA, 22 bp, Artificial sequence
  Synthetic nucleotide
cattcagtgcgacgcatctcag

SEQ ID NO: 28 - DNA, 18 bp, Artificial sequence
  Synthetic nucleotide
agaccgtcatcttctccc

SEQ ID NO: 29 - DNA, 20 bp, Artificial sequence
  Synthetic nucleotide
atctcagctcggtatcaata

SEQ ID NO: 30 - DNA, 19 bp, Artificial sequence
  Synthetic nucleotide
ctataagcctgatcgtgtc

SEQ ID NO: 31 - DNA, 39 bp, Artificial sequence
  Synthetic nucleotide
gacacgatcaggcttatagatggactataaggatcacga

SEQ ID NO: 32 - DNA, 39 bp, Artificial sequence
  Synthetic nucleotide
tattgataccgagctgagattcacccgccaactttcctc

SEQ ID NO: 33 - DNA, 25 bp, Artificial sequence
  Synthetic nucleotide
atggagccctctgcagagatccccc

SEQ ID NO: 34 - DNA, 28 bp, Artificial sequence
  Synthetic nucleotide
tatggaggagggacctgttatgatggtg

SEQ ID NO: 35 - DNA, 19 bp, Artificial sequence
  Synthetic nucleotide
atggcaaacatcaacatcc

SEQ ID NO: 36 - DNA, 22 bp, Artificial sequence
  Synthetic nucleotide
ctataagcctgatcgtgtcttc

SEQ ID NO: 37 - DNA, 18 bp, Artificial sequence
  Synthetic nucleotide
atggtgagcaagggcgag

SEQ ID NO: 38 - DNA, 16 bp, Artificial sequence
  Synthetic nucleotide
cttgtacagctcgtcc

(19) United States
(12) Patent Application Publication
(10) Pub. No.: US 2024/0182916 A1
(43) Pub. Date: Jun. 6, 2024

(54) RECOMBINANT VIRAL EXPRESSION VECTORS AND METHODS OF USE

(71) Applicant: North Carolina State University, Raleigh, NC (US)

(72) Inventors: Anna E. Whitfield, Raleigh, NC (US); Surapathrudu Kanakala, Raleigh, NC (US); Kathleen M. Martin, Raleigh, NC (US)

(21) Appl. No.: 18/551,562

(22) PCT Filed: Mar. 25, 2022
(86) PCT No.: PCT/US22/21944

§ 371 Date: Sep. 20, 2023

Related U.S. Application Data
(60) Provisional application No. 63/166,608, filed on Mar. 26, 2021.

(51) Int. Cl. C12N 15/82; C07K 14/005; C12N 9/22; C12N 15/86

(52) U.S. Cl. CPC C12N 15/8203; C07K 14/005; C12N 9/22; C12N 15/86; C12N 2310/20; C12N 2760/20022; C12N 2760/20043

(57) ABSTRACT

The present disclosure provides materials and methods relating to recombinant viral expression vectors. In particular, the present disclosure provides novel recombinant viral expression vectors comprising an RNA virus backbone that encodes a virus capable of infecting a target organism and expressing a polynucleotide-of-interest in that target organism. The novel viral vector constructs provided herein are a versatile expression tool for interrogating gene function and an efficient delivery platform for gene editing technology.

### CROSS REFERENCE TO RELATED APPLICATIONS

This application claims priority to and the benefit of U.S. Provisional Patent Application No. 63/166,608 filed Mar. 26, 2021, which is incorporated herein by reference in its entirety for all purposes.

### GOVERNMENT SUPPORT

This invention was made with government support under HR0011-17-2-0054 awarded by the Defense Advanced Research Projects Agency. The government has certain rights in the invention.

### SEQUENCE LISTING

The text of the computer readable sequence listing filed herewith, titled “39355-601_SEQUENCE_LISTING_ST25”, created Mar. 23, 2022, having a file size of 238,574 bytes, is hereby incorporated by reference in its entirety

### FIELD

The present disclosure provides materials and methods relating to recombinant viral expression vectors. In particular, the present disclosure provides novel recombinant viral expression vectors comprising an RNA virus backbone that encodes a virus capable of infecting a target organism and expressing a polynucleotide-of-interest in that target organism. The novel viral vector constructs provided herein are a versatile expression tool for interrogating gene function and an efficient delivery platform for gene editing technology.

### BACKGROUND

Plant viruses have been demonstrated as multifaceted recombinant vectors to carry sequences for virus induced gene silencing, heterologous protein expression and genome editing in both dicotyledonous and monocotyledonous plants. However, efficient, stable, and systemic expression of large foreign proteins in monocot plants and insect pests is challenging due to genetic instability. Rhabdoviruses are a diverse family of negative strand RNA viruses (NSVs) that infect vertebrates, invertebrates, and plants. Plant NSVs can be segmented and unsegmented viruses and cause significant crop yield loss. Plant unsegmented viruses are currently classified into four genera, Cytorhabdoviruses that replicate in the cytoplasm and three nuclear replicating genera Alphanucleorhabdovirus, Betaucleorhabdovirus, and Gammanucleorhabdovirus. One genus of plant rhabdoviruses, Dichorhavirus, is bipartite with the genome divided into two RNAs. All rhabdoviruses antigenomic RNA (agRNA) genomes consists of 5′ leader and 3′ trailer sequences encoding five viral structural protein genes organised in the order and separated by conserved gene junctions [5″-nucleoprotein (N), phosphoprotein (P), matrix protein (M), glycoprotein (G), and viral polymerase (L)-3′] (Jackson and Li, 2016, Walker et al., 2011). The rhabdovirus genomes are complementary to their mRNA and require the minimal infection unit (genomic RNA , N, P and L proteins) for initiation of virus transcription and replication. Unlike animal rhabdoviruses, plant adapted viruses have at least one additional protein that facilitates virus movement from cell to cell.

For positive strand RNA viruses, the encapsidated viral genome can function both as a template for genome replication and serves as RNA (mRNA) and can deliver their genomics RNAs directly to cellular ribosomes for translation. Viral proteins expressed after infection can bind selectively to viral (+) RNA, which probably leads to the recruitment of the viral RNA from translation to replication. In the past few decades, virologists have developed many infectious clones for positive-stranded RNA viruses. This strategy involved generation of in vitro RNA transcripts using promoters (bacteriophage T7 or SP6, cytomegalovirus promoter for animal viruses and modified 35S or duplicated 35S for plant viruses). Constructing NSR virus infectious clones has not become routine due to several factors, including a large genome size about 11-15 kb in size; the characteristic that NSR virus genome are not infectious after introduction into susceptible cells because neither genomic or antigenomic RNAs serve as an mRNA; they generally require ribonucleoproteins (RNP's) and functional polymerase (L) in the same cell for both transcription and replication; and they generally require plasmids encoding the N, P, and L proteins, full length viral genome, and viral suppressors of silencing.

The first infectious clones for NSRs were developed for the animal rhabdoviruses Rabies virus and Vesicular stomatitis virus (VSV). Cells were cotransfected with positive strand or antigenomic RNA (agRNA) and viral replication proteins (N, P and L) transcription was facilitated by bacteriophage T7 polymerase. In case of plant negative strand RNA viruses, the path for generating infectious virus from cDNA clones required overcoming additional difficulties. First, the lack of continuous cell cultures for plant NSVs made this technology more challenging. Second, the relative rigidity of the plant cell wall hinders the entry of multiple plasmids into a single cell, a requirement for launching plant NSVs that requires the full-length genome and replication proteins (N, P, and L). Third, all plant NSV infectious clones to date require co-expressing viral RNA silencing suppressors (VSRs: tomato bushy stunt virus p19, tobacco etch virus HC-Pro and barley stripe mosaic virus γb proteins) that interfere with host RNA silencing to enhance RNPs in trans. Fourth, the viral sequences are often unstable in Escherichia coli and Agrobacterium tumefaciens either due to spontaneous insertions or deletions or prokaryotic promoter-like elements in the viral genomes. More recently, rescue of first plant rhabdoviruses has been achieved for, namely Sonchus yellow net virus (SYNV) in Nicotiana benthamiana and barley yellow striate mosaic virus (BYSMV) in barley by co-expressing RNPs and viral suppressors. Additionally, infectious clones of segmented RNA viruses in the order Bunyavirales, tomato spotted wilt virus (TSWV) and Rose rosette virus (RRV), have been established.

### SUMMARY

Embodiments of the present disclosure include a recombinant viral expression vector. In accordance with these embodiments, the vector includes a negative strand RNA virus (NSV) backbone comprising polynucleotide sequences encoding core proteins, and at least one expression cassette comprising an exogenous polynucleotide sequence flanked by viral gene junctions from the NSV backbone. In some embodiments, the NSV backbone encodes a virus capable of infecting a target organism and expressing the exogenous polynucleotide sequence.

In some embodiments, the core proteins comprise a nucleoprotein (N), a phosphoprotein (P), a matrix protein (M), a glycoprotein (G), and a polymerase protein (L). In some embodiments, the N protein is upstream of the other core proteins. In some embodiments, the L protein is downstream of the other core proteins.

In some embodiments, the L protein comprises at least one intronic sequence. In some embodiments, the at least one intronic sequence enhances stability of the vector. In some embodiments, the at least one intronic sequence is inserted into the L protein at a splice site or a predicted splice site. In some embodiments, the at least one intronic sequence is heterologous.

In some embodiments, the at least one expression cassette is positioned between the N protein and the P protein. In some embodiments, the viral gene junctions flanking the at least one expression cassette are N/P junctions.

In some embodiments, the vector comprises at least a second expression cassette comprising a second exogenous polynucleotide sequence. In some embodiments, the second expression cassette is flanked by N/P junctions.

In some embodiments, the first exogenous polynucleotide sequence encodes a first gene-of-interest. In some embodiments, the second exogenous polynucleotide sequence encodes a second gene-of-interest. In some embodiments, the first or the second exogenous polynucleotide encodes a gene editing protein. In some embodiments, the gene editing protein is selected from the group consisting of: Cas1, Cas2, Cas3, Cas4, Cas5, Cas6, Cas7, Cas8, Cas9, Cas10, Cas12, and Cas13. In some embodiments, the first or the second exogenous polynucleotide encodes at least one guide RNA (gRNA). In some embodiments, the first or the second exogenous polynucleotide encodes a fluorescent protein.

In some embodiments, the first or the second exogenous polynucleotide encodes at least one component of a CRISPR-Cas system. In some embodiment, the CRISPR-Cas system is a genome engineering system, a CRISPRa system, a CRISPRi system, a base editing system, a prime editing system, or a gap editing system. In some embodiments, the CRISPR-Cas system is selected from the group consisting of a Type I CRISPR-Cas system, a Type II CRISPR-Cas system, a Type III CRISPR-Cas system, and a Type V CRISPR-Cas system. In some embodiments, the system is a Type I CRISPR-Cas system, and the at least one component is Cas3. In some embodiments, the system is a Type II CRISPR-Cas system, and the at least one component is Cas9. In some embodiments, the system is a Type III CRISPR-Cas system, and the at least one component is Csm (III-A) or Cmr (III-B). In some embodiments, the system is a Type V CRISPR-Cas system, and the at least one component is Cas12a.

In some embodiments, the vector further comprises a promoter and/or a terminator.

In some embodiments, the NSV backbone is derived from a virus from the genus Alphanucleorhabdovirus.

In some embodiments, target organism is a plant. In some embodiments, the plant is selected from the group consisting of maize, wheat, potato, caneberry, eggplant, pepper, tomato, potato, sorghum, rice, taro, alfalfa, peanut, black currant, barley, oat, broccoli, cabbage, kale, lettuce, papaya, strawberry, coffee, citrus, orchid, and pulses (beans and lentils), and variants thereof. In some embodiments, target organism is an insect. In some embodiments, the vector is transmissible to insects.

Embodiments of the present disclosure also include a cell comprising any of the vectors described herein. In some embodiments, the cell is selected from the group consisting of a plant cell, an insect cell, a yeast cell, and a bacterial cell.

### BRIEF DESCRIPTION OF THE DRAWINGS

FIGS. 1A-1E: Rescue of recombinant MMV-GFP virus in maize and leafhopper. (A-D) Schematic diagrams of the pJL-MMV, pTF-N and P, pJL-L-intron plasmids. A) The full length MMV-plasmid designed for transcription to yield the MMV antigenome (ag) RNA and contains the full-length MMV cDNA position between a truncated CaMV double 35S promoter (2X35S) and the hepatitis delta (HDV) ribozyme sequence in pJL89 binary plasmid. B) The full length ORF of N and P were inserted between the 2X35S promoter and 35S terminator sequences in pTF binary plasmid. C) The full length ORF of L with plant intron ST-LS1 inserted in between 2X35S and 35S terminator sequences in pJL89 binary plasmid. D) The full length pJL-MMV-GFP contains duplicate N/P gene junctions flanking the green fluorescent protein (GFP) between the N and P genes of maize mosaic virus (MMV) antigenome cDNA. Note that the sequences are shown in antigenomic (mRNA) sense. E) Illustration of the MMV rescue procedure in maize and planthoppers.

FIGS. 2A-2B: Schematic diagrams of MMV-based expression vectors. A) The full length pJL-MMV-Cry51Aa2-GFP contains duplicate N/P gene junctions flanking the Bacillus thuringiensis crystal protein (Cry51Aa2) between the N and P genes of maize mosaic virus (MMV) antigenome cDNA. B) The full length pJL-MMV-Hvt-ACA-GFP contains duplicate N/P gene junctions flanking the onion lectin (Allium cepa agglutinin; ACA) and Hadronyche versuta (Hvt) coding sequences between the N and P genes of maize mosaic virus (MMV) antigenome cDNA. Note that the sequences are shown in antigenomic (mRNA) sense.

FIGS. 3A-3D: Engineering MMV vectors for delivery of CRISPR-Cas9 for maize and planthopper-targeted gene mutagenesis. A-D) schematic representation of MMV genome with gRNA and Cas9 expression strategies. A) pJL-MMV-tRNA-g[ZmTrxH-ZmVPP1]-GFP contain sequence elements in the order “N/P gene junction-tRNA^{Gly}-Zea mays Thioredoxin H (ZmTrxH) gRNA-tRNA^{Gly}-Zea mays Thioredoxin H (ZmTrxH) gRNA- tRNA^{Gly}- N/P gene junction-green fluorescent protein (GFP)-N/P gene junction” inserted in between the N and P genes of maize mosaic virus (MMV) antigenome cDNA. B) pJL-MMV-tRNA-g[Pmcn]-GFP contain sequence elements in the order “N/P gene junction-tRNA^{Gly}-P. maidis cinnabar (Pmcn) gRNA1-tRNA^{Gly}-P. maidis cinnabar (Pmcn) gRNA3-tRNA^{Gly}-N/P gene junction-green fluorescent protein (GFP)-N/P gene junction” inserted in between the N and P genes of maize mosaic virus (MMV) antigenome cDNA. C) pJL-MMV-tRNA-g[ZmPDS]-Cas9-GFP contain sequence elements in the order “N/P gene junction-tRNA^{Gly}-Zea mays phytoene desaturase (ZmPDS) gRNA-tRNA^{Gly}-N/P gene junction-Cas9-N/P gene junction-green fluorescent protein (GFP)-N/P gene junction” inserted in between the N and P genes of maize mosaic virus (MMV) antigenome cDNA. D) pJL-MMV-tRNA-g[ZmLHT1]-Cas9-GFP contain sequence elements in the order “N/P gene junction-tRNA^{Gly}-Zea mays phytoene desaturase (ZmLHT1) gRNA-tRNA^{Gly}-N/P gene junction-Cas9-N/P gene junction-green fluorescent protein (GFP)-N/P gene junction” inserted in between the N and P genes of maize mosaic virus (MMV) antigenome cDNA. Note that the sequences are shown in antigenomic (mRNA) sense.

FIGS. 4A-4B: Expression of GFP engineered into MMV. A) Foci of GFP in N. benthamiana leaves after agroinfiltration with Agrobacterium strains containing NPL alone, VSRs alone, MMV alone, MMV-GFP alone and MMV-GFP+NPL+VSRs. GFP foci were photographed at 14 days post infiltration (dpi) with a fluorescence microscope (Scale bar: 1000 um). B) Immunoblot analysis of the expression of MMV N (48.67 kDa) and P (30.4 kDa) in agroinfiltrated leaves using N, P specific antibodies. Lanes: M: Mock, MMV: pJL-MMV+NPL+VSR and MMV-GFP: pJL-MMV-GFP+NPL+VSR. Numbers from on the top panel represent samples from different plants (M=Mock, MMV (lanes 1-3) and MMV-GFP (lanes 4-8)). C) Immunoblot analysis of the expression of GFP in pJL-MMV-GFP+pTFN&P+pJL-L and VSRs agroinfiltrated leaves using GFP specific antibodies at 25 dpi. Lanes: M: Mock, MMV-GFP: pJL-MMV-GFP+NPL+VSR and MMV: pJL-MMV+NPL+VSR. Numbers from on the top panel represent samples from different plants (M=Mock and MMV-GFP (lanes 1-4)).

FIGS. 5A-5C: Maize diseased symptoms vascular puncture inoculated with MMV-GFP virions. A) Maize systemic leaves showing mosaic symptoms after 24 days post inoculation (dpi) inoculated with MMV-GFP infected N. benthamiana sap by vascular puncture method. Maize seeds were inoculated with crude extracts of the agroinfiltrated N. benthamiana leaves by vascular puncture inoculation (VPI). Foci of the maize leaves expressing GFP in the infected plants. GFP foci were photographed at 24 dpi with a fluorescence microscope (Scale bar: 1000 μm). Mock plants inoculated with pJL-MMV-GFP alone did not show any virus symptoms or GFP. B) Immunoblot analysis of the expression of GFP (32.5 kDa) in symptomatic systemic maize leaves using GFP specific antibodies. Lanes: M: Mock, MMV: pJL-MMV+NPL+VSR and MMV-GFP: pJL-MMV-GFP+NPL+VSR and RubL: Coomassie blue stained Rubisco large subunit (RubL) was used a loading control. Numbers from 1 to 8 on the top panel represent samples from different plants (M=Mock, MMV (lanes 1-3) and MMV-GFP (lanes 4-8)). C) Transmission electron micrographs of thin sections of MMV-GFP infected maize showed abundant bacilliform particles in the nucleus and cytoplasm. The black arrows show the virions in the cytoplasm. Scale bars, 2 μm and 1 μm.

FIG. 6: GFP expressed from MMV-GFP infections in the progeny nymphs after acquisition access feeding on healthy and infected plants. P. maidis nymphs feeding on MMV-GFP infected maize plants at 14 dpi were photographed with a fluorescence microscope. Bars, 1000 μm.

FIGS. 7A-7B: Time course observation of GFP expression in MMV-GFP infected systemic maize systemic leaves. A) GFP foci were photographed at 1 week to 8th week post inoculation with a fluorescence microscope (Scale bar: 1000 μm). Maize plants inoculated with MMV alone did not show green florescence. B) Stability of GFP sequence carried by MMV vectors. RT-PCR analysis of plants inoculated with MMV-GFP. MMV N and GFP primers to detect the virus presence and stability of the GFP insertion and Zmactin was used as internal control.

FIG. 8: GFP foci were photographed at 24 dpi with a fluorescence microscope (Scale bar: 1000 μm). Mock plants inoculated with pJL-MMV-GFP/pJL-MMV-Pmcn-GFP/pJL-MMV-TrxH1-VPP1-GFP +NPL+VSRs.

### DETAILED DESCRIPTION

Newly enabled reverse genetics technologies allow researchers to genetically manipulate the genomes of plant negative strand RNA viruses (NSVs) to elucidate gene function. Reverse genetics systems for plant NSVs have had technical difficulties due to lack of appropriate systems for encapsidation of genomic RNA, e.g., assembling infectious nucleocapsids in vitro from multiple components and their genetic instability. Embodiments of the present disclosure include a construction of a plant negative RNA virus infectious clone. The full-length infectious clone was established based on agrobacterium-mediated delivery of full-length maize mosaic virus (MMV) antigenomic RNA and co-expression of the core proteins required for viral transcription and replication (nucleoprotein (N), phosphoprotein (P), large polymerase (L)) and viral suppressors of RNA silencing in N. benthamiana. Insertion of intron 2 ST-SL1 into the polymerase gene, L, increased the stability of the infectious clone in Escherichia coli and Agrobacterium tumefaciens. To monitor virus infection in vivo, a GFP gene was inserted in between the N and P gene junctions to generate recombinant MMV-GFP. Agroinfiltrated cDNA clones of MMV and MMV-GFP were able to replicate in single cells of N. benthamiana and could then be inoculated into maize and insects, robust virus replication occurred in both hosts. Additionally, uniform systemic infection of maize and high levels of reporter gene expression was observed. The insect vectors were able to support virus infection and transmission when virus was injected into their bodies or orally delivered by feeding on infected plants. The reporter gene showed high levels of expression and stability over three cycles of transmission in plants and insects. Taken together, the embodiments of the present disclosure represent the first infectious clone of an alphanucleorhabdovirus, which will be a versatile expression tool for expression of foreign proteins in maize and planthoppers.

Section headings as used in this section and the entire disclosure herein are merely for organizational purposes and are not intended to be limiting.

### 1. DEFINITIONS

Unless otherwise defined, all technical and scientific terms used herein have the same meaning as commonly understood by one of ordinary skill in the art. In case of conflict, the present document, including definitions, will control. Preferred methods and materials are described below, although methods and materials similar or equivalent to those described herein can be used in practice or testing of the present disclosure. All publications, patent applications, patents and other references mentioned herein are incorporated by reference in their entirety. The materials, methods, and examples disclosed herein are illustrative only and not intended to be limiting.

The terms “comprise(s),” “include(s),” “having,” “has,” “can,” “contain(s),” and variants thereof, as used herein, are intended to be open-ended transitional phrases, terms, or words that do not preclude the possibility of additional acts or structures. The singular forms “a,” “and” and “the” include plural references unless the context clearly dictates otherwise. The present disclosure also contemplates other embodiments “comprising,” “consisting of” and “consisting essentially of,” the embodiments or elements presented herein, whether explicitly set forth or not.

For the recitation of numeric ranges herein, each intervening number there between with the same degree of precision is explicitly contemplated. For example, for the range of 6-9, the numbers 7 and 8 are contemplated in addition to 6 and 9, and for the range 6.0-7.0, the number 6.0, 6.1, 6.2, 6.3, 6.4, 6.5, 6.6, 6.7, 6.8, 6.9, and 7.0 are explicitly contemplated.

“Correlated to” as used herein refers to compared to.

As used herein, the term “nucleic acid molecule” refers to any nucleic acid containing molecule, including but not limited to, DNA or RNA. The term encompasses sequences that include any of the known base analogs of DNA and RNA including, but not limited to, 4-acetylcytosine, 8-hydroxy-N6-methyladenosine, aziridinylcytosine, pseudoisocytosine, 5-(carboxyhydroxylmethyl) uracil, 5-fluorouracil, 5-bromouracil, 5-carboxymethylaminomethyl-2-thiouracil, 5-carboxymethylaminomethyluracil, dihydrouracil, inosine, N6-isopentenyladenine, 1-methyladenine, 1-methylpseudouracil, 1-methylguanine, 1-methylinosine, 2,2-dimethylguanine, 2-methyladenine, 2-methylguanine, 3-methylcytosine, 5-methylcytosine, N6-methyladenine, 7-methylguanine, 5-methylaminomethyluracil, 5-methoxyaminomethyl-2-thiouracil, beta-D-mannosylqueosine, 5′-methoxycarbonylmethyluracil, 5-methoxyuracil, 2-methylthio-N6-isopentenyladenine, uracil-5-oxyacetic acid methylester, uracil-5-oxyacetic acid, oxy butoxosine, pseudouracil, queosine, 2-thiocytosine, 5-methyl-2-thiouracil, 2-thiouracil, 4-thiouracil, 5-methyluracil, N-uracil-5-oxyacetic acid methylester, uracil-5-oxyacetic acid, pseudouracil, queosine, 2-thiocytosine, and 2,6-diaminopurine.

The term “gene” refers to a nucleic acid (e.g., DNA) sequence that comprises coding sequences for the production of a polypeptide, precursor, or RNA (e.g., rRNA, tNA, sRNA, microRNA, lincRNA). The polypeptide can be encoded by a full-length coding sequence or by any portion of the coding sequence so long as the desired activity or functional properties (e.g., enzymatic activity, ligand binding, signal transduction, immunogenicity, etc.) of the full-length or fragment are retained. The term also encompasses the coding region of a structural gene and the sequences located adjacent to the coding region on both the 5′ and 3′ ends for a distance of about 1 kb or more on either end such that the gene corresponds to the length of the full-length mRNA. Sequences located 5′ of the coding region and present on the mRNA are referred to as 5′ non-translated sequences. Sequences located 3′ or downstream of the coding region and present on the mRNA are referred to as 3′ non-translated sequences. The term “gene” encompasses both cDNA and genomic forms of a gene. A genomic form or clone of a gene contains the coding region interrupted with non-coding sequences termed “introns” or “intervening regions” or “intervening sequences.” Introns are segments of a gene that are transcribed into nuclear RNA (hnRNA); introns may contain regulatory elements such as enhancers. Introns are removed or “spliced out” from the nuclear or primary transcript; introns therefore are absent in the messenger RNA (mRNA) transcript. The mRNA functions during translation to specify the sequence or order of amino acids in a nascent polypeptide.

As used herein, the term “heterologous gene” refers to a gene that is not in its natural environment. For example, a heterologous gene includes a gene from one species introduced into another species. A heterologous gene also includes a gene native to an organism that has been altered in some way (e.g., mutated, added in multiple copies, linked to non-native regulatory sequences, etc.). Heterologous genes are distinguished from endogenous genes in that the heterologous gene sequences are typically joined to DNA sequences that are not found naturally associated with the gene sequences in the chromosome or are associated with portions of the chromosome not found in nature (e.g., genes expressed in loci where the gene is not normally expressed).

As used herein, a “double-stranded nucleic acid” may be a portion of a nucleic acid, a region of a longer nucleic acid, or an entire nucleic acid. A “double-stranded nucleic acid” may be, e.g., without limitation, a double-stranded DNA, a double-stranded RNA, a double-stranded DNA/RNA hybrid, etc. A single-stranded nucleic acid having secondary structure (e.g., base-paired secondary structure) and/or higher order structure comprises a “double-stranded nucleic acid”. For example, triplex structures are considered to be “double-stranded”. In some embodiments, any base-paired nucleic acid is a “double-stranded nucleic acid”

The term “single-stranded” oligonucleotides generally refers to those oligonucleotides that contain a single covalently linked series of nucleotide residues.

The terms “oligomers” or “oligonucleotides” include RNA or DNA sequences of more than one nucleotide in either single chain or duplex form and specifically includes short sequences such as dimers and trimers, in either single chain or duplex form, which can be intermediates in the production of the specifically binding oligonucleotides. “Modified” forms used in candidate pools contain at least one non-native residue. “Oligonucleotide” or “oligomer” is generic to polydeoxyribonucleotides (containing 2′-deoxy-D-ribose or modified forms thereof), such as DNA, to polyribonucleotides (containing D-ribose or modified forms thereof), such as RNA, and to any other type of polynucleotide which is an N-glycoside or C-glycoside of a purine or pyrimidine base, or modified purine or pyrimidine base or abasic nucleotides. Oligonucleotide” or “oligomer” can also be used to describe artificially synthesized polymers that are similar to RNA and DNA, including, but not limited to, oligos of peptide nucleic acids (PNA).

As used herein, a “non-native” nucleic acid sequence refers to a nucleic acid sequence not normally present in a bacterium, e.g., an extra copy of an endogenous sequence. or a heterologous sequence such as a sequence from a different species, strain, or substrain of bacteria, or a sequence that is modified and/or mutated as compared to the unmodified sequence from bacteria of the same subtype. In some embodiments, the non-native nucleic acid sequence is a synthetic, non-naturally occurring sequence. The non-native nucleic acid sequence may be a regulatory region, a promoter, a gene, and/or one or more genes in a gene cassette. In some embodiments, “non-native” refers to two or more nucleic acid sequences that are not found in the same relationship to each other in nature. The non-native nucleic acid sequence may be present on a plasmid or chromosome. In addition, multiple copies of any regulatory region, promoter, gene, and/or gene cassette may be present in the bacterium, wherein one or more copies of the regulatory region, promoter, gene, and/or gene cassette may be mutated or otherwise altered as described herein. In some embodiments, the genetically engineered bacteria are engineered to comprise multiple copies of the same regulatory region, promoter, gene, and/or gene cassette in order to enhance copy number or to comprise multiple different components of a gene cassette performing multiple different functions.

As used herein, “promoter” refers to a nucleotide sequence that is capable of controlling the expression of a coding sequence or gene. Promoters are generally located 5′ of the sequence that they regulate. Promoters may be derived in their entirety from a native gene, or be composed of different elements derived from promoters found in nature, and/or comprise synthetic nucleotide segments. Those skilled in the art will readily ascertain that different promoters may regulate expression of a coding sequence or gene in response to a particular stimulus, e.g., in a cell- or tissue- specific manner, in response to different environmental or physiological conditions, or in response to specific compounds. Prokaryotic promoters are typically classified into two classes: inducible and constitutive.

The term “isolated” when used in relation to a nucleic acid, as in “an isolated oligonucleotide” or “isolated polynucleotide” refers to a nucleic acid sequence that is identified and separated from at least one component or contaminant with which it is ordinarily associated in its natural source. Isolated nucleic acid is such present in a form or setting that is different from that in which it is found in nature. In contrast, non-isolated nucleic acids as nucleic acids such as DNA and RNA found in the state they exist in nature. For example, a given DNA sequence (e.g., a gene) is found on the host cell chromosome in proximity to neighboring genes; RNA sequences, such as a specific mRNA sequence encoding a specific protein, are found in the cell as a mixture with numerous other mRNAs that encode a multitude of proteins. However, isolated nucleic acid encoding a given protein includes, by way of example, such nucleic acid in cells ordinarily expressing the given protein where the nucleic acid is in a chromosomal location different from that of natural cells, or is otherwise flanked by a different nucleic acid sequence than that found in nature. The isolated nucleic acid, oligonucleotide, or polynucleotide may be present in single-stranded or double-stranded form. When an isolated nucleic acid, oligonucleotide or polynucleotide is to be utilized to express a protein, the oligonucleotide or polynucleotide will contain at a minimum the sense or coding strand (the oligonucleotide or polynucleotide may be single-stranded), but may contain both the sense and anti-sense strands (the oligonucleotide or polynucleotide may be double-stranded).

As used herein, the term “purified” or “to purify” refers to the removal of components (e.g., contaminants) from a sample. For example, antibodies are purified by removal of contaminating non-immunoglobulin proteins; they are also purified by the removal of immunoglobulin that does not bind to the target molecule. The removal of non-immunoglobulin proteins and/or the removal of immunoglobulins that do not bind to the target molecule results in an increase in the percent of target-reactive immunoglobulins in the sample. In another example, recombinant polypeptides are expressed in bacterial host cells and the polypeptides are purified by the removal of host cell proteins; the percent of recombinant polypeptides is thereby increased in the sample. The term “substantially purified” as used herein refers to a molecule such as a polypeptide, carbohydrate, nucleic acid etc. which is substantially free of other proteins, lipids, carbohydrates or other materials with which it is naturally associated. One skilled in the art can purify viral or bacterial polypeptides using standard techniques for protein purification. The substantially pure polypeptide will often yield a single major band on a non-reducing polyacrylamide gel. In the case of partially glycosylated polypeptides or those that have several start codons, there may be several bands on a non-reducing polyacrylamide gel, but these will form a distinctive pattern for that polypeptide. The purity of the viral or bacterial polypeptide can also be determined by amino-terminal amino acid sequence analysis. Other types of antigens such as polysaccharides, small molecule, mimics etc. are included within the present disclosure.

“Peptide” and “polypeptide” as used herein, and unless otherwise specified, refer to polymer compounds of two or more amino acids joined through the main chain by peptide amide bonds (—C(O)NH—). The term “peptide” typically refers to short amino acid polymers (e.g., chains having fewer than 25 amino acids), whereas the term “polypeptide” typically refers to longer amino acid polymers (e.g., chains having more than 25 amino acids).

As used herein, the term “fragment” refers to a peptide or polypeptide that results from dissection or “fragmentation” of a larger whole entity (e.g., protein, polypeptide, enzyme, etc.), or a peptide or polypeptide prepared to have the same sequence as such. Therefore, a fragment is a subsequence of the whole entity (e.g., protein. polypeptide, enzyme, etc.) from which it is made and/or designed. A peptide or polypeptide that is not a subsequence of a preexisting whole protein is not a fragment (e.g., not a fragment of a preexisting protein).

As used herein, the term “sequence identity” refers to the degree two polymer sequences (e.g., peptide, polypeptide, nucleic acid, etc.) have the same sequential composition of monomer subunits. The term “sequence similarity” refers to the degree with which two polymer sequences (e.g., peptide, polypeptide, nucleic acid, etc.) have similar polymer sequences. For example, similar amino acids are those that share the same biophysical characteristics and can be grouped into the families, e.g., acidic (e.g., aspartate, glutamate), basic (e.g., lysine, arginine, histidine), non-polar (e.g., alanine, valine, leucine, isoleucine, proline, phenylalanine, methionine, tryptophan) and uncharged polar (e.g., glycine, asparagine, glutamine, cysteine, serine, threonine, tyrosine). The “percent sequence identity” (or “percent sequence similarity”) is calculated by: (1) comparing two optimally aligned sequences over a window of comparison (e.g., the length of the longer sequence, the length of the shorter sequence, a specified window), (2) determining the number of positions containing identical (or similar) monomers (e.g., same amino acids occurs in both sequences, similar amino acid occurs in both sequences) to yield the number of matched positions, (3) dividing the number of matched positions by the total number of positions in the comparison window (e.g., the length of the longer sequence, the length of the shorter sequence, a specified window), and (4) multiplying the result by 100 to yield the percent sequence identity or percent sequence similarity. For example, if peptides A and B are both 20 amino acids in length and have identical amino acids at all but 1 position, then peptide A and peptide B have 95% sequence identity. If the amino acids at the non-identical position shared the same biophysical characteristics (e.g., both were acidic), then peptide A and peptide B would have 100% sequence similarity. As another example, if peptide C is 20 amino acids in length and peptide D is 15 amino acids in length, and 14 out of 15 amino acids in peptide D are identical to those of a portion of peptide C, then peptides C and D have 70% sequence identity, but peptide D has 93.3% sequence identity to an optimal comparison window of peptide C. For the purpose of calculating “percent sequence identity” (or “percent sequence similarity”) herein, any gaps in aligned sequences are treated as mismatches at that position.

In some embodiments the substitutions can be conservative amino acid substitutions. Examples of conservative amino acid substitutions, unlikely to affect biological activity, include the following: alanine for serine, valine for isoleucine, aspartate for glutamate, threonine for serine, alanine for glycine, alanine for threonine, serine for asparagine, alanine for valine, serine for glycine, tyrosine for phenylalanine, alanine for proline, lysine for arginine, aspartate for asparagine, leucine for isoleucine, leucine for valine, alanine for glutamate, aspartate for glycine, and these changes in the reverse. See e.g., Neurath et al., The Proteins, Academic Press, New York (1979), the relevant portions of which are incorporated herein by reference. Further, an exchange of one amino acid within a group for another amino acid within the same group is a conservative substitution, where the groups are the following: (1) alanine, valine, leucine, isoleucine, methionine, norleucine, and phenylalanine: (2) histidine, arginine, lysine, glutamine, and asparagine; (3) aspartate and glutamate; (4) serine, threonine, alanine, tyrosine, phenylalanine, tryptophan, and cysteine; and (5) glycine, proline, and alanine.

The term “homology” and “homologous” refers to a degree of identity. There may be partial homology or complete homology. A partially homologous sequence is one that is less than 100% identical to another sequence.

As used herein, the terms “complementary” or “complementarity” are used in reference to polynucleotides (e.g., a sequence of nucleotides such as an oligonucleotide or a target nucleic acid) related by the base-pairing rules. For example, for the sequence “5′-A-G-T-3′” is complementary to the sequence “3′-T-C-A-5′.” Complementarity may be “partial,” in which only some of the nucleic acids' bases are matched according to the base pairing rules. Or, there may be “complete” or “total” complementarity between the nucleic acids. The degree of complementarity between nucleic acid strands has significant effects on the efficiency and strength of hybridization between nucleic acid strands. This is of particular importance in amplification reactions, as well as detection methods that depend upon binding between nucleic acids. Either term may also be used in reference to individual nucleotides, especially within the context of polynucleotides. For example, a particular nucleotide within an oligonucleotide may be noted for its complementarity, or lack thereof, to a nucleotide within another nucleic acid strand, in contrast or comparison to the complementarity between the rest of the oligonucleotide and the nucleic acid strand.

In some contexts, the term “complementarity” and related terms (e.g., “complementary”, “complement”) refers to the nucleotides of a nucleic acid sequence that can bind to another nucleic acid sequence through hydrogen bonds, e.g., nucleotides that are capable of base pairing, e.g., by Watson-Crick base pairing or other base pairing. Nucleotides that can form base pairs, e.g., that are complementary to one another, are the pairs: cytosine and guanine, thymine and adenine, adenine and uracil, and guanine and uracil. The percentage complementarity need not be calculated over the entire length of a nucleic acid sequence. The percentage of complementarity may be limited to a specific region of which the nucleic acid sequences that are base-paired, e.g., starting from a first base-paired nucleotide and ending at a last base-paired nucleotide. The complement of a nucleic acid sequence as used herein refers to an oligonucleotide which, when aligned with the nucleic acid sequence such that the 5′ end of one sequence is paired with the 3′ end of the other, is in “antiparallel association.” Certain bases not commonly found in natural nucleic acids may be included in the nucleic acids of the present disclosure and include, for example, inosine and 7-deazaguanine. Complementarity need not be perfect; stable duplexes may contain mismatched base pairs or unmatched bases. Those skilled in the art of nucleic acid technology can determine duplex stability empirically considering a number of variables including, for example, the length of the oligonucleotide, base composition and sequence of the oligonucleotide, ionic strength and incidence of mismatched base pairs.

Thus, in some embodiments, “complementary” refers to a first nucleobase sequence that is at least 60%, 65%, 70%, 75%, 80%, 85%, 90%, 95%, 97%, 98%, or 99% identical to the complement of a second nucleobase sequence over a region of 8, 9, 10, 11, 12, 13, 14, 15, 16, 17, 18, 19, 20, 21, 22, 23, 24, 25, 30, 35, 40, 45, 50, 55, 60, 65, 70, 75, 80, 85, 90, 95, 100, or more nucleobases, or that the two sequences hybridize under stringent hybridization conditions. “Fully complementary” means each nucleobase of a first nucleic acid is capable of pairing with each nucleobase at a corresponding position in a second nucleic acid. For example, in certain embodiments, an oligonucleotide wherein each nucleobase has complementarity to a nucleic acid has a nucleobase sequence that is identical to the complement of the nucleic acid over a region of 8, 9, 10, 11, 12, 13, 14, 15, 16, 17, 18, 19, 20, 21, 22, 23, 24, 25, 30, 35, 40, 45, 50, 55, 60, 65, 70, 75, 80, 85, 90, 95, 100, or more nucleobases.

The term “plant” as used herein encompasses a whole plant, a grafted plant, ancestor(s) and progeny of the plants and plant parts, including seeds, shoots, stems, roots (including tubers), rootstock, scion, and plant cells, tissues and organs. The plant may be in any form including suspension cultures, embryos, meristematic regions, callus tissue, leaves, gametophytes, sporophytes, pollen, and microspores. According to some embodiments of the present disclosure, plants targeted with the vectors described herein include, but are not limited to, maize, wheat, potato, caneberry, eggplant, pepper, tomato, potato, sorghum, rice, taro, alfalfa, peanut, black currant, barley, oat, broccoli, cabbage, kale, lettuce, papaya, strawberry, coffee, citrus, orchid, and pulses (beans and lentils), and any variants thereof.

According to some embodiments of the present disclosure the plant is a dicotyledonous plant. According to some embodiments of the present disclosure the plant is a monocotyledonous plant. According to some embodiments of the present disclosure, there is provided a plant cell exogenously expressing the polynucleotide of some embodiments of the present disclosure, the nucleic acid construct of some embodiments of the present disclosure and/or the polypeptide of some embodiments of the present disclosure.

According to some embodiments of the present disclosure, expressing the exogenous polynucleotide of the present disclosure within the plant is affected by transforming one or more cells of the plant with the exogenous polynucleotide, followed by generating a mature plant from the transformed cells and cultivating the mature plant under conditions suitable for expressing the exogenous polynucleotide within the mature plant.

According to some embodiments of the present disclosure, the transformation is performed by introducing to the plant cell a nucleic acid construct which includes the exogenous polynucleotide of some embodiments of the present disclosure and at least one promoter for directing transcription of the exogenous polynucleotide in a host cell (a plant cell). Further details of suitable transformation approaches are provided herein.

As mentioned, the nucleic acid construct according to some embodiments of the present disclosure comprises a promoter sequence and the isolated polynucleotide of some embodiments of the present disclosure. According to some embodiments of the present disclosure, the isolated polynucleotide is operably linked to the promoter sequence. A coding nucleic acid sequence is “operably linked” to a regulatory sequence (e.g., promoter) if the regulatory sequence is capable of exerting a regulatory effect on the coding sequence linked thereto.

### 2. VECTORS, COMPOSITIONS, AND METHODS

Genome editing is revolutionizing crop improvement capabilities, providing a path for increasing plant resistance to biotic and abiotic stressors and improving other agronomic traits. One challenge for this system is effective delivery of the guide RNAs (gRNAs) to target the gene of interest and expression of large proteins to execute the editing process (i.e., CRISPR associated protein (Cas)) in advanced breeding lines or commercial varieties of specialty crops that are resistant to transformation. Plant rhabdoviruses can be genetically manipulated to carry multiple foreign genes and they have the capacity to carry large nucleic acid sequences that are not feasible with other plant viruses. Plant rhabdoviruses are diverse and common, and with the increase in sequencing of plant viromes, numerous viruses have been identified that infect major and minor crop plants such as maize, wheat, potato, coffee, papaya, caneberry, etc. As described further herein, new viral vectors have been developed using the plant rhabdovirus, maize mosaic virus (MMV). These recombinant viruses exhibited stable expression of a reporter gene in maize and in the insect that naturally transmits the virus, the corn planthopper. Additionally, the recombinant viruses have been engineered to express foreign genes, including gRNAs and CRISPR components in plants and insects.

Thus, embodiments of the present disclosure include various recombinant viral expression vectors useful for targeted genetic manipulation in plants and insects. In accordance with these embodiments, the vectors include a negative strand RNA virus (NSV) backbone comprising polynucleotide sequences encoding core proteins, and at least one expression cassette comprising an exogenous polynucleotide sequence flanked by viral gene junctions from the NSV backbone. In some embodiments, the NSV backbone encodes a virus capable of infecting a target organism and expressing the exogenous polynucleotide sequence.

In some embodiments, the core proteins comprise a nucleoprotein (N), a phosphoprotein (P), a matrix protein (M), a glycoprotein (G), and a polymerase protein (L). In some embodiments, the N protein is upstream of the other core proteins. In some embodiments, the L protein is downstream of the other core proteins. Polynucleotide sequences encoding other exogenous and/or endogenous proteins can also be included in the vectors of the present disclosure, in addition to these core proteins.

In some embodiments, the L protein comprises at least one intronic sequence. In some embodiments, the at least one intronic sequence enhances stability of the vector. In some embodiments, the at least one intronic sequence is inserted into the L protein at a splice site or a predicted splice site. In some embodiments, the at least one intronic sequence is heterologous.

In some embodiments, the at least one expression cassette is positioned between any of the core proteins. In some embodiments, the at least one expression cassette is positioned between the N protein and the P protein. In some embodiments. the viral gene junctions flanking the at least one expression cassette are the gene junctions found between two of the core proteins. In some embodiments, the viral gene junctions flanking the expression cassette are the gene junctions found between N/P junctions.

In some embodiments, the vector includes at least a second expression cassette comprising a second exogenous polynucleotide sequence. In some embodiments, the viral gene junctions flanking the second expression cassette are the gene junctions found between two of the core proteins. In some embodiments, the second expression cassette is flanked by N/P junctions. In some embodiments, the vector includes at least a third, fourth, fifth, sixth, seventh, eighth, ninth, or tenth expression cassette comprising a third, fourth, fifth, sixth, seventh. eighth, ninth, or tenth exogenous polynucleotide sequence.

In some embodiments, the first exogenous polynucleotide sequence encodes a first gene-of-interest. In some embodiments, the second exogenous polynucleotide sequence encodes a second gene-of-interest. In some embodiments, the first or the second exogenous polynucleotide encodes a gene editing protein. In some embodiments, the gene editing protein is selected from the group consisting of: Cas1, Cas2, Cas3, Cas4, Cas5, Cas6, Cas7, Cas8, Cas9, Cas10, Cas12, and Cas13. In some embodiments, the first or the second exogenous polynucleotide encodes at least one guide RNA (gRNA). In some embodiments, the first or the second exogenous polynucleotide encodes a fluorescent protein. In some embodiments, the first or the second exogenous polynucleotide encodes at least one component of a CRISPR-Cas system. In some embodiments, the CRISPR-Cas system is selected from the group consisting of a Type I CRISPR-Cas system, a Type II CRISPR-Cas system, a Type III CRISPR-Cas system, and a Type V CRISPR-Cas system. In some embodiments, the system is a Type I CRISPR-Cas system, and the at least one component is Cas3. In some embodiments, the system is a Type II CRISPR-Cas system, and the at least one component is Cas9. In some embodiments, the system is a Type III CRISPR-Cas system, and the at least one component is Csm (III-A) or Cmr (III-B). In some embodiments, the system is a Type V CRISPR-Cas system, and the at least one component is Cas12a.

In some embodiments, the vector further comprises a promoter and/or a terminator. In some embodiments, the vector comprises any of the features listed in Table 1.

In some embodiments, the NSV backbone is derived from a virus from the genus Alphanucleorhabdovirus. In some embodiments, the NSV backbone is derived from a virus from the genus Alphanucleorhabdovirus, including but not limited to, the following: Eggplant mottled dwarf alphanucleorhabdovirus; Maize Iranian mosaic alphanucleorhabdovirus; Maize mosaic alphanucleorhabdovirus; Morogoro maize-associated alphanucleorhabdovirus; Physostegia chlorotic mottle alphanucleorhabdovirus; Potato yellow dwarf alphanucleorhabdovirus; Rice yellow stunt alphanucleorhabdovirus; Taro vein chlorosis alphanucleorhabdovirus; and Wheat yellow striate alphanucleorhabdovirus.

In some embodiments, the NSV backbone is derived from a virus from the genus Cytorhabdovirus. In some embodiments, the NSV backbone is derived from a virus from the genus Cytorhabdovirus, including but not limited to, the following: Alfalfa dwarf cytorhabdovirus; Barley yellow striate mosaic cytorhabdovirus; Broccoli necrotic yellows cytorhabdovirus; Cabbage cytorhabdovirus; Colocasia bobone disease-associated cytorhabdovirus; Festuca leaf streak cytorhabdovirus; Lettuce necrotic yellows cytorhabdovirus; Lettuce yellow mottle cytorhabdovirus; Maize yellow striate cytorhabdovirus; Maize-associated cytorhabdovirus; Northern cereal mosaic cytorhabdovirus; Papaya cytorhabdovirus; Persimmon cytorhabdovirus; Raspberry vein chlorosis cytorhabdovirus; Rice stripe mosaic cytorhabdovirus; Sonchus cytorhabdovirus 1; Strawberry crinkle cytorhabdovirus; Tomato yellow mottle-associated cytorhabdovirus; Wheat American striate mosaic cytorhabdovirus; Wuhan 4 insect cytorhabdovirus; Wuhan 5 insect cytorhabdovirus; Wuhan 6 insect cytorhabdovirus; and Yerba mate chlorosis-associated cytorhabdovirus.

In some embodiments, the NSV backbone is derived from a virus from the genus Betanucleorhabdovirus. In some embodiments, the NSV backbone is derived from a virus from the genus Betanucleorhabdovirus, including but not limited to, the following: Alfalfa betanucleorhabdovirus; Blackcurrant betanucleorhabdovirus; Datura yellow vein betanucleorhabdovirus; Sonchus yellow net betanucleorhabdovirus; Sowthistle yellow vein betanucleorhabdovirus; and Trefoil betanucleorhabdovirus.

In some embodiments, the NSV backbone is derived from a virus from the genus Gammanucleorhabdovirus. In some embodiments, the NSV backbone is derived from a virus from the genus Gammanucleorhabdovirus, including but not limited to, Maize fine streak gammanucleorhabdovirus.

In some embodiments, the NSV backbone is derived from a virus from the genus Dichorhavirus. In some embodiments, the NSV backbone is derived from a virus from the genus Dichorhavirus, including but not limited to, the following: Citrus chlorotic spot dichorhavirus; Citrus leprosis N dichorhavirus; Clerodendrum chlorotic spot dichorhavirus; Coffee ringspot dichorhavirus; and Orchid fleck dichorhavirus

In some embodiments, target organism is a plant. In some embodiments, the plant is selected from the group consisting of maize, wheat, potato, caneberry, eggplant, pepper, tomato, potato, sorghum, rice, taro, alfalfa, peanut, black currant, barley, oat, broccoli, cabbage, kale, lettuce, papaya, strawberry, coffee, citrus, orchid, and pulses (beans and lentils), and variants thereof. In some embodiments, target organism is an insect. In some embodiments, the viral vector is transmissible to insects.

The viral vector constructs of the present disclosure can be used with, and/or adapted for use with, prokaryotic cells, eukaryotic cells, and plant cells. In some embodiments, the cell is a mammalian cell. The present disclosure also provides an isolated cell comprising any of the components or systems described herein. Exemplary cells can include those that can be easily and reliably grown, have reasonably fast growth rates, have well characterized expression systems, and can be transformed or transfected easily and efficiently. Examples of suitable prokaryotic cells include, but are not limited to, cells from the genera Bacillus (such as Bacillus subtilis and Bacillus brevis), Clostridia (such as Clostridium difficile or Clostridium autoethanogenum), Escherichia (such as E. coli), Lactobacilli, Klebsiella, Myxobacteria, Pseudomonas, Streptomyces, Salmonella, Vibrio (such as Vibrio cholerae or Vibrio nutrifaciens) and Envinia. Suitable eukaryotic cells are known in the art and include, for example, yeast cells, insect cells, and mammalian cells. Examples of suitable yeast cells include those from the genera Kluyveromyces, Pichia, Rhino-sporidium, Saccharomyces, and Schizosaccharomyces. Exemplary insect cells include Sf-9 and HIS (Invitrogen, Carlsbad, Calif.) and are described in, for example, Kitts et al., Biotechniques, 14: 810-817 (1993); Lucklow, Curr. Opin. Biotechnol., 4: 564-572 (1993); and Lucklow et al., J. Virol., 67: 4566-4579 (1993). Examples of suitable plant cell lines are derived from plants such as Arabidopsis (such as the Landsberg erecta cell line), sugarcane, pea, tobacco (such as the BY-2 cell line). maize, wheat, potato, caneberry, eggplant, pepper, tomato, potato, sorghum, rice, taro, alfalfa, peanut, black currant, barley, oat, broccoli, cabbage, kale, lettuce, papaya, strawberry, coffee, citrus, orchid, and pulses (beans and lentils), and variants thereof.

In some embodiments, the cell can also be a cell that is used for therapeutic purposes. The cell can be a mammalian cell, and in some embodiments, the cell is a human cell. A number of suitable mammalian and human cells are known in the art, and many are available from the American Type Culture Collection (ATCC, Manassas, Va.). Examples of suitable mammalian cells include, but are not limited to, Chinese hamster ovary cells (CHO) (ATCC No. CCL61), CHO DHFR-cells (Urlaub et al., Proc. Natl. Acad. Sci. USA, 97: 4216-4220 (1980)), human embryonic kidney (HEK) 293 or 293T cells (ATCC No. CRL1573), and 3T3 cells (ATCC No. CCL92). Other suitable mammalian cell lines are the monkey COS-1 (ATCC No. CRL1650) and COS-7 cell lines (ATCC No. CRL1651), as well as the CV-1 cell line (ATCC No. CCL70). Further exemplary mammalian cells include primate, rodent, and human cell lines, including transformed cell lines. Normal diploid cells, cell strains derived from in vitro culture of primary tissue, as well as primary explants, are also suitable. Other suitable mammalian cell lines include, but are not limited to, mouse neuroblastoma N2A cells, HeLa, HEK, A549, HepG2, mouse L-929 cells, and BHK or Hak hamster cell lines. Methods for selecting suitable cells and methods for transformation, culture, amplification, screening, and purification of cells are known in the art.

Embodiments of the present disclosure also include a cell comprising any of the vectors described herein. In some embodiments, the cell is selected from the group consisting of a plant cell, an insect cell, a yeast cell, and a bacterial cell.

In accordance with the above, the recombinant vector constructs of the present disclosure can include, but are not limited to, the various features listed below in Table 1.

TABLE 1
Exemplary features of the recombinant vector constructs.

| | Short name | | | | |
|---|---|---|---|---|---|
| | used in the | | | | |
| Features in the | genome | | Plasmids containing this | | Sequence in |
| plasmid | maps | Function | feature | | color |
| Left boarder T- | LB T-DNA | 23 base pair direct | 1. | pJL-MMV (16, 774 bp) | Green |
| DNA repeat | repeat | repeats occur at the | 2. | pTF-N&P (12, 338 bp) | |
| | | ends of the two | 3. | pJL-L-intron (10, 493 bp) | |
| | | adjacent T-DNAs | 4. | pJL-MMV-GFP (17, 677 | |
| | | in the pJL89 and | | bp) | |
| | | pTF binary | 5. | pJL-MMV-Cry51Aa2- | |
| | | plasmids: | | GFP (18, 861 bp) | |
| | | promotes T-DNA | 6. | pJL-MMV-Hvt-ACA- | |
| | | transfer and | | GFP (18, 635 bp) | |
| | | integration in one | 7. | pJJL-MMV-tRNA- | |
| | | direction | | g[ZmTrxH-ZmVPP1]- | |
| | | | | GFP (18, 285 bp) | |
| | | | 8. | pJL-MMV-tRNA-Pmcn- | |
| | | | | GFP (18, 285 bp) | |
| | | | 9. | pJL-MMV-tRNA-PDS- | |
| | | | | Cas9-GFP (22, 551 bp) | |
| | | | 10. | pJL-MMV-tRNA- | |
| | | | | ZmLHT1-Cas9-GFP (22, | |
| | | | | 551 bp). | |
| Right border T- | RB- T-DNA | 23 base pair direct | 1. | pJL-MMV (16, 774 bp) | Light green |
| DNA repeat | repeat | repeats occur at the | 2. | pTF-N&P (12, 338 bp) | |
| | | ends of the two | 3. | pJL-L-intron (10, 493 bp) | |
| | | adjacent T-DNAs | 4. | pJL-MMV-GFP (17, 677 | |
| | | in the pJL89 and | | bp) | |
| | | pTF binary | 5. | pJL-MMV-Cry51Aa2- | |
| | | plasmids: | | GFP (18, 861 bp) | |
| | | promotes T-DNA | 6. | pJL-MMV-Hvt-ACA- | |
| | | transfer and | | GFP (18, 635 bp) | |
| | | integration in one | 7. | pJJL-MMV-tRNA- | |
| | | direction | | g[ZmTrxH-ZmVPP1]- | |
| | | | | GFP (18, 285 bp) | |
| | | | 8. | pJL-MMV-tRNA-Pmcn- | |
| | | | | GFP (18, 285 bp) | |
| | | | 9. | pJL-MMV-tRNA-PDS- | |
| | | | | Cas9-GFP (22, 551 bp) | |
| | | | 10. | pJL-MMV-tRNA- | |
| | | | | ZmLHT1-Cas9-GFP (22, | |
| | | | | 551 bp). | |
| 35S promoter | 35S | 35S Cauliflower | 1. | pJL-MMV (16, 774 bp) | Red |
| | | Mosaic virus | 2. | pTF-N&P (12, 338 bp) | |
| | | (CaMV) promoter | 3. | pJL-L-intron (10, 493 bp) | |
| | | is used to secure | 4. | pJL-MMV-GFP (17, 677 | |
| | | transgene | | bp) | |
| | | expression and it | 5. | pJL-MMV-Cry51Aa2- | |
| | | facilitates high | | GFP (18, 861 bp) | |
| | | level transcription | 6. | pJL-MMV-Hvt-ACA- | |
| | | in a wide range of | | GFP (18, 635 bp) | |
| | | plants. | 7. | pJJL-MMV-tRNA- | |
| | | | | g[ZmTrxH-ZmVPP1]- | |
| | | | | GFP (18, 285 bp) | |
| | | | 8. | pJL-MMV-tRNA-Pmcn- | |
| | | | | GFP (18, 285 bp) | |
| | | | 9. | pJL-MMV-tRNA-PDS- | |
| | | | | Cas9-GFP (22, 551 bp) | |
| | | | 10. | pJL-MMV-tRNA- | |
| | | | | ZmLHT1-Cas9-GFP (22, | |
| | | | | 551 bp). | |
| Terminator | Term | Termination | 1. | pJL-MMV (16, 774 bp) | Orange accent 2 |
| | | sequence of the | 2. | pTF-N&P (12, 338 bp) | |
| | | nopaline synthase | 3. | pJL-L-intron (10, 493 bp) | |
| | | gene, isolated from | 4. | pJL-MMV-GFP (17, 677 | |
| | | Agrobacterium | | bp) | |
| | | tumefaciens. The | 5. | pJL-MMV-Cry51Aa2- | |
| | | function of this | | GFP (18, 861 bp) | |
| | | sequence is to | 6. | pJL-MMV-Hvt-ACA- | |
| | | signal the | | GFP (18, 635 bp) | |
| | | termination of the | 7. | pJJL-MMV-tRNA- | |
| | | gene expression. | | g[ZmTrxH-ZmVPP1]- | |
| | | | | GFP (18, 285 bp) | |
| | | | 8. | pJL-MMV-tRNA-Pmcn- | |
| | | | | GFP (18, 285 bp) | |
| | | | 9. | pJL-MMV-tRNA-PDS- | |
| | | | | Cas9-GFP (22, 551 bp) | |
| | | | 10. | pJL-MMV-tRNA- | |
| | | | | ZmLHT1-Cas9-GFP (22, | |
| | | | | 551 bp). | |
| Plasmid | trfA | Required for | 1. | pJL-MMV (16, 774 bp) | Dark blue |
| replication | | initiation of | 2. | pJL-L-intron (10, 493 bp) | |
| initiator protein | | plasmid DNA | 3. | pJL-MMV-GFP (17, 677 | |
| | | replication along | | bp) | |
| | | with host-derived | 4. | pJL-MMV-Cry51Aa2- | |
| | | DnaA and other | | GFP (18, 861 bp) | |
| | | host proteins. | 5. | pJL-MMV-Hvt-ACA- | |
| | | | | GFP (18, 635 bp) | |
| | | | 6. | pJJL-MMV-tRNA- | |
| | | | | g[ZmTrxH-ZmVPP1]- | |
| | | | | GFP (18, 285 bp) | |
| | | | 7. | pJL-MMV-tRNA-Pmcn- | |
| | | | | GFP (18, 285 bp) | |
| | | | 8. | pJL-MMV-tRNA-PDS- | |
| | | | | Cas9-GFP (22, 551 bp) | |
| | | | 9. | pJL-MMV-tRNA- | |
| | | | | ZmLHT1-Cas9-GFP (22, | |
| | | | | 551 bp). | |
| Kanamycin | KanR | Kanamycin gene | 1. | pJL-MMV (16, 774 bp) | Purple |
| Resistance | | in the binary | 2. | pJL-L-intron (10, 493 bp) | |
| | | plasmids to easily | 3. | pJL-MMV-GFP (17, 677 | |
| | | detect plasmid | | bp) | |
| | | containing bacteria | 4. | pJL-MMV-Cry51Aa2- | |
| | | when cells grown | | GFP (18, 861 bp) | |
| | | under selective | 5. | pJL-MMV-Hvt-ACA- | |
| | | media. | | GFP (18, 635 bp) | |
| | | | 6. | pJJL-MMV-tRNA- | |
| | | | | g[ZmTrxH-ZmVPP1]- | |
| | | | | GFP (18, 285 bp) | |
| | | | 7. | pJL-MMV-tRNA-Pmcn- | |
| | | | | GFP (18, 285 bp) | |
| | | | 8. | pJL-MMV-tRNA-PDS- | |
| | | | | Cas9-GFP (22, 551 bp) | |
| | | | 9. | pJL-MMV-tRNA- | |
| | | | | ZmLHT1-Cas9-GFP (22, | |
| | | | | 551 bp). | |
| Origin of | OriV | The replication of | 1. | pJL-MMV (16, 774 bp) | Gold |
| replication | | origin of plasmids | 2. | pJL-L-intron (10, 493 bp) | |
| | | pJL89 at which | 3. | pJL-MMV-GFP (17, 677 | |
| | | replication is | | bp) | |
| | | initiated and helps | 4. | pJL-MMV-Cry51Aa2- | |
| | | to make additional | | GFP (18, 861 bp) | |
| | | copies. | 5. | pJL-MMV-Hvt-ACA- | |
| | | | | GFP (18, 635 bp) | |
| | | | 6. | pJJL-MMV-tRNA- | |
| | | | | g[ZmTrxH-ZmVPP1]- | |
| | | | | GFP (18, 285 bp) | |
| | | | 7. | pJL-MMV-tRNA-Pmcn- | |
| | | | | GFP (18, 285 bp) | |
| | | | 8. | pJL-MMV-tRNA-PDS- | |
| | | | | Cas9-GFP (22, 551 bp) | |
| | | | 9. | pJL-MMV-tRNA- | |
| | | | | ZmLHT1-Cas9-GFP (22, | |
| | | | | 551 bp). | |
| Tobacco etch virus | TEV | The RNA genome | 1. | pTF-N&P (12, 338 bp) | Lavender |
| (TEV) enhancer | enhancer | of tobacco etch | | | |
| | | virus (TEV), a | | | |
| | | plant potyvirus, | | | |
| | | helps high level | | | |
| | | expression of | | | |
| | | proteins in the | | | |
| | | binary plasmid. | | | |
| Solanum | Intron ST- | Intron 2 of the | 1. | pJL-MMV (16, 774 bp) | Light Blue |
| tuberosum intron 2 | LS1 | light-inducible | 2. | pJL-L-intron (10, 493 bp) | |
| | | gene ST-LS1 | 3. | pJL-MMV-GFP (17, 677 | |
| | | (NCBI accession | | bp) | |
| | | number X04753) | 4. | pJL-MMV-Cry51Aa2- | |
| | | from Solanum | | GFP (18, 861 bp) | |
| | | tuberosum. MMV | 5. | pJL-MMV-Hvt-ACA- | |
| | | L contains an | | GFP (18, 635 bp) | |
| | | intron derived | 6. | pJJL-MMV-tRNA- | |
| | | from the | | g[ZmTrxH-ZmVPP1]- | |
| | | intervening | | GFP (18, 285 bp) | |
| | | sequence 2 (IV2) | 7. | pJL-MMV-tRNA-Pmcn- | |
| | | of the potato ST- | | GFP (18, 285 bp) | |
| | | LS1 gene. | 8. | pJL-MMV-tRNA-PDS- | |
| | | | | Cas9-GFP (22, 551 bp) | |
| | | | 9. | pJL-MMV-tRNA- | |
| | | | | ZmLHT1-Cas9-GFP (22, | |
| | | | | 551 bp). | |

In accordance with the above embodiments, the viral vectors of the present disclosure can be used to express one or more components of a CRISPR-Cas system, for example, to edit the genome of a target organism, to place a tag an endogenous gene in a target organism, to regulate the expression of a target gene (e.g., CRISPRi or CRISPRa), and to facilitate any variations genetic manipulation in a target organism. According to the structure and function of Cas protein, CRISPR/Cas systems can be categorized into two classes (class I, class II), which are further subdivided into six types (type I-VI). Class I includes type I, III, and IV, and class II includes type II, V, and VI. Type I, II, and V systems recognize and cleave DNA, type VI can edit RNA, and type III edits both DNA and RNA. Differences between Types I, II, III, and V CRISPR-Cas systems are provided below in Table 2 (see, e.g., Liu et al., Microbial Cell Factories vol. 19: 172 (2020)).

TABLE 2
Components of Type I, II, III, and V CRISPR-Cas systems.

| Classification | Type I | Type II | Type III | Type V |
|---|---|---|---|---|
| Signature | Cas3 | Cas9 | Csm | Cas12a |
| protein | (or | (1368 | (III-A) or | (1200-1300 |
| | Cas3) | amino | Cmr | amino acids) |
| | | acids) | (III-B) | |
| Effector | Cascade | crRNA and | Cascade | crRNA |
| | | tracrRNA | | |
| | | (sgRNA) | | |
| PAM | 3-nt | G-rich | Without | 5′-YTN-3′ |
| sequence | | sequence, | PAM | (FnCas12a), |
| | | 5′-NGG-3′ | | 5′-TTTN-3′ |
| | | | | (AsCas12a, |
| | | | | LbCas12a) |
| Cleavage | SSBs | DSB | SSBs at | DSB (Sticky |
| product | | (flat | every | end with |
| | | end) | 6-nt | 5 nucleotides |
| | | | | protruding) |

By delivering one or more components of a CRISPR-Cas system into a target organism, the viral vectors of the present disclosure can be used for genome editing, CRISPR interference to inhibit or repress gene expression (CRISPRi), CRISPR activation to activate or enhance gene expression (CRISPRa), and specific base editing (e.g., using base editors, gap editors, and prime editors).

### 3. MATERIALS AND METHODS

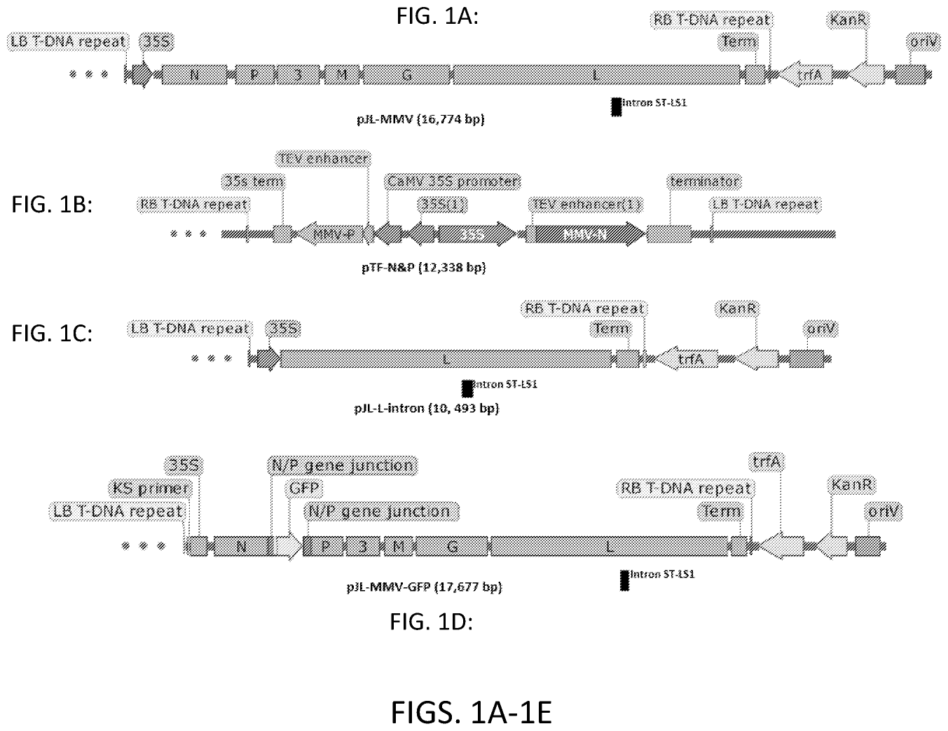

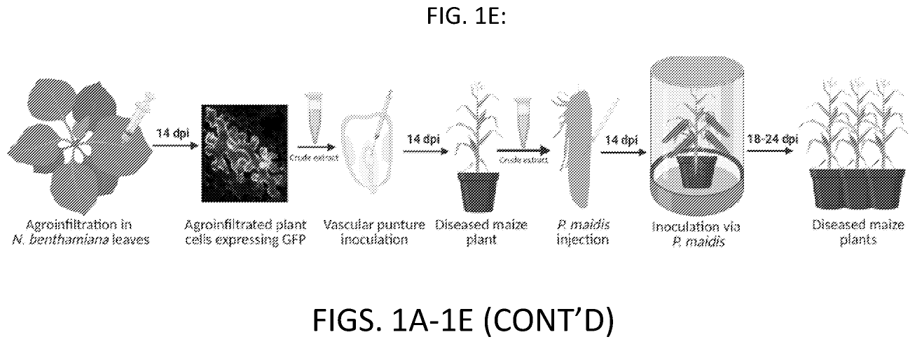

Construction of MMV infectious clone and introduction of intron into L gene. The full-length cDNA of MMV (pbrick-MMV, 12,170 bp; accession number MK828539) was synthesized in pccl-pbrick plasmid de novo by GenScript. In this study, full length MMV was synthesized in pbrick plasmid to launch the virus into maize plants directly by biolistic method. The full length MMV cDNA was synthesized between cauliflower mosaic virus promoter (CaMV) 35S promoter and CaMV PolyA terminator. Full length MMV and derivatives were subcloned in pJL89 and pTF binary vectors for agroinfiltration in N. benthamiana and synthesised all virus sequences in pUC57 from GenScript. To generate pJL-MMV, fragments 12,205 bp and 4137 bp were amplified from pbrick-MMV and pJL89 using pJL-MMV F/R and pJL F/R primers. respectively. The forward primer contains 17 nt overhangs (lowercase) complementary to the 3′ end of the 35S promoter. whereas reverse primer contains 18 nt overhangs (lowercase) complementary to the 5′ end of the self-cleaving hepatitis delta virus (HDV) ribozyme, respectively. The two overlapping fragments were amplified using Q5 high fidelity polymerase and introduced into the pJL89 vector by one-step Hifi assembly (NEB) (FIG. 1A). To generate pTF-N&P (plasmid for expression of protein components of the replication complex), MMV N and P cDNAs were amplified using MMV NF/NR and PF/PR primers from pbrick-MMV plasmid and subcloned into pTF binary plasmid between double CaMV 35S promoter and NOS terminator using Gateway Clonase enzyme mix (Invitrogen) (FIG. 1B). To generate pJL-L for expression of the polymerase. two overlapping PCR fragments 6936 bp and 4137 bp were amplified from pbrick-MMV and pJL89 using 35SLF/TerLR and pJL F&R primers, respectively (FIG. 1C). Competent cells of E. coli strain Top 10, DH10B, DH5α (ThermoFisher Scientific, USA) and NEB Stable Competent E. coli (High Efficiency, NEB) were used for cloning steps. Except pTF-N&P, all the competent cells mentioned above tended to lose the full length MMV and L gene plasmid in overnight cultures at three different temperatures (25° C., 30° C. and 37° C.).

To increase the stability of the plasmid, intron 2 (189 bp) of the light-inducible gene ST-LS1 (X04753) from Solanum tuberosum was introduced into the large polymerase gene (L) between 3281 and 3282 splice site (CTGCGGACAG\hat{ }GTATCGATAT; SEQ ID NO: 11) nucleotides. The putative intron splicing sites of wild type L gene sequences was predicted by Alternative Splice Site Predictor (ASSP) (Wang and Marin. 2006). To generate pUC-MMV-L-intron (6784 bp), MMV L gene from 2893 nucleotides- ST-LS1-pJL89 terminator in pUC57 was synthesized de novo by GenScript. To generate pJL-L-intron, three divergent overlapping PCR fragments LF1 (2,943 bp) and LF2 (3,487 bp) and third fragment pJL89 (LF3, 4,137 bp) were amplified from pbrick-MMV, pUC-MMV-L-intron and pJL89 plasmids respectively (FIG. 1C). LF1, LF2 and LF3 fragments were amplified with the primer pairs 35SLF/MMVLXSR; LXSF/TerLR and TerLF/pJLR respectively. To generate pJL-MMV-intron, three divergent overlapping PCR fragments were (MF1 (3,489 bp), MF2 (9,224 bp) and MF3 (4,137 bp) were amplified from pUC-MMV-L-intron, pbrick-MMV and pJL89 plasmids, respectively. MF1, MF2 and MF3 fragments were amplified using LXSF/TerLF; pJLMMVF/MMVLXSR and TerLF and pJLR primers pairs. respectively. The purified fragments overlapping fragments were further assembled using Hifi assembly (NEB). The resulting pJL-L-intron was cloned in a binary vector pJL89 between double 35S promoter (2X35S) at 5′ terminus and HDV ribozyme at the 3′ terminus. All the fragments (MF1, MF2 and MF3) were amplified using Q5 high fidelity polymerase and introduced into binary plasmids using Hifi assembly (NEB) (FIG. 1A). When the E. coli transformants were cultured overnight, pJL-L and pJL-MMV with plant intron showed single plasmid with expected band with PCR and restriction digestion. Primers used for Hifi assembly (NEB) contain a 10-15 nt overhangs at its 5′ end that is homologous to the 5′ end of another PCR product, so that the two PCR products could be ligated and circularized. All PCRs were carried out using Q5 high fidelity polymerase (New England Biolabs). NEB Stable Competent E. coli (High Efficiency, NEB) were used in these cloning steps. Primers used for Hifi assembly (NEB) contain a 10-15 nt overhangs at its 5′ end that is homologous to the 5′ end of another PCR product, so that the two PCR products could be ligated and circularized. All PCRs were carried out using Q5 high fidelity polymerase (New England Biolabs).

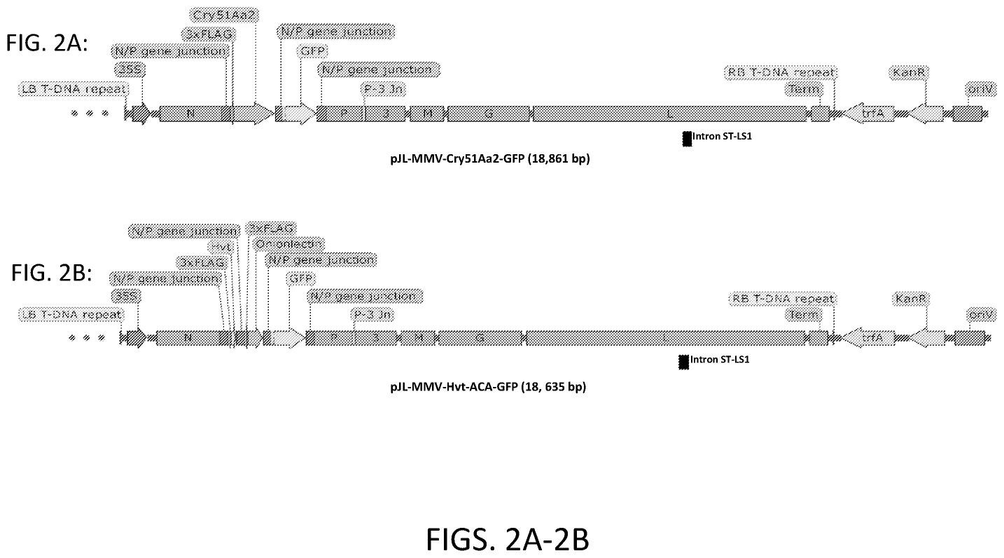

To develop a recombinant MMV vector for foreign gene expression in plants, a duplicated N/P gene junction sequence along with the enhanced green fluorescent protein (eGFP), Bacillus thuringiensis crystal protein (Cry51Aa2), onion lectin (Allium cepa agglutinin; ACA) and Hadronyche versuta (Hvt) coding sequences were inserted into the pMMV plasmid between the N and P genes to generate pJL-MMV-GFP. In this configuration, GFP mRNA synthesis is initiated immediately after termination of the upstream N protein mRNA synthesis by the duplicated N/P gene junction and is followed by P mRNA synthesis that is directed by the native N/P gene junction. To construct the pJL-MMV-GFP, pJL-MMV-Cry and pJL-MMV-Hvt-ACA plasmid for generation of recombinant MMV with a GFP, Cry51Aa2 and Hvt-ACA expression cassettes inserted between the N and P ORF, a unique Bsu36I site was used just before the start codon of the P ORF. The complete coding region of GFP, Cry51Aa2 and Hvt-ACA, followed by the N/P gene junction sequence, was synthesised by GenScript. To facilitate sub-cloning, an Bsu36I site was introduced at either ends of the GFP and N/P gene junction clone. Both pJL-MMV and pUC57-GFP-N/P plasmids were digested by Bsu36I and ligated to generate pJL-MMV-GFP (FIG. 1D). Similarly. pUC57-Cry51Aa2-GFP-N/P and pUC57-Hvt-ACA-GFP-N/P were digested with Bsu36I and ligated to Bsu36I linearized pJL-MMV to generate pJL-MMV-Cry51Aa2-GFP (FIG. 2A) and pJL-MMV-Hvt-ACA-GFP (FIG. 2B) plasmids. Clones with correctly oriented GFP-N/P, Cry51Aa2-GFP-N/P and Hvt-ACA-GFP-N/P inserts were verified by MMV1355F and MMVPR primers and by sequencing. The plasmid agroinfiltrated into N. benthamiana leaves along with the bacterial mixture harbouring the pTF-N&P, pJL-MMV-L and VSR plasmids. All constructs were confirmed by sequencing and transformed into Agrobacterium tumefaciens GV3101.

Agrobacterium infiltration. Virus infections on N. benthamiana was achieved by Agrobacterium-mediated transient gene expression of infectious constructs from the T-DNA of a binary plasmid pJL89. A. tumefaciens GV3101 with pJL-MMV, pTF-N&P and pJL-L plasmids were individually added to 50 ml of Luria-broth containing plasmid specific Kanamycin (50 μg/ml) or Spectinomycin (50 μg/ml) and rifampicin (50 μg/ml) and Gentamicin (50 μg/ml). Cells grown overnight were harvested by centrifugation and resuspended in the infiltration buffer described in Kanakala et al., 2019 and incubated for 3 h at room temperature in the presence of 100 μM acetosyringone. After incubation, equal volumes of Agrobacterium cultures harbouring the pJL-MMV or pJL-MMV-GFP alone and pJL-MMV or pJL-MMV-GFP+NPL and VSR's coinfiltrated into the N. benthamiana. In the case of pJL-MMV-GFP, pTF-N&P and pJL-L culture at 0.5 O.D and VSRs at OD₆₀₀ 0.2 were used for agroinfiltration.

Image acquisition. The fluorescence of plant leaves and insects was observed with the objective of 4× using the BioTek slide reader and Image Software. version 3.04. Images were captured using default settings. Total GFP fluorescence was measured by Gen5 software (BioTek, Winooski, VT. USA). Leaves agroinfiltrated with pJL alone were used as a negative control.

Vascular puncture inoculation, injections and insect transmission studies. GFP expressing N. benthamiana leaf extracts infiltrated with MMV-GFP plasmids were homogenised in extraction buffer (10 mM Potassium buffer; pH 7) and centrifuged at 12,000 g for 5 min at 4° C. Then 5 ul of crude supernatants were vascular puncture inoculated using a 5 pin-tattoo needle into the overnight soaked maize kernels at 30° C. Then the VPI inoculated seeds were incubated in a humid tray at 30° C. for two days before sowing. These plants were subsequently observed for systemic symptom expression or image analysis. The symptomatic plant leaves showing mosaic symptoms and GFP expression were homogenized in extraction buffer ((100 mM Tris-HCl, 10 mM Mg (CH3COO)₂, 1 mM MnCl₂, and 40 mM Na₂SO₃, pH 8.4) and centrifuged at 12,000 g for 10 min at 4° C. (Jackson & Wagner, 1998). Then, 40 nl of the crude extract supernatants were injected into the thoraxes of anesthetized P. maidis (males and females) using a Nanoinject III Programmable Nanoliter injector (Drummond Scientific Co., Broomall, PA, USA). The surviving injected adults (˜300) were maintained on the healthy maize seedlings for 7 days and then transferred to healthy maize seedlings for a 2-week inoculation period and these experiments were repeated twice. Extraction buffer alone injected adults (˜30/replicate) were used for control experiments. Symptoms in plants and GFP expression in systemic leaves and insects were observed and recorded periodically.

For analysis of acquisition and transmission efficiency via an oral acquisition route, P. maidis were given a 10 days acquisition access period on VPI inoculated maize plants. These insects were then released in groups of four per plant on one week old maize plants (n=15) for a 2-week inoculation access period. P. maids, which were allowed to feed on healthy maize plants served as a negative control (n=15). P. maidis transmission experiments from maize to maize were conducted, and the development of symptoms was scored at different time intervals.

Immuno-blot analysis. For western blot analysis. proteins were separated on 4-15% gradient Mini-PROTEAN TGX Precast gels (Bio-Rad, Catalog number 4561084). Western blot analysis of the protein extracts was performed according to the procedures described previously by Caplan et al., (2008). Proteins were transferred to nitrocellulose membranes by using the Trans-Blot Turbo Transfer system (Bio-Rad). Membranes were blocked using 5% non-fat dry milk for 1 h then washed in 3× in Tris-buffered saline containing 0.1% Tween-20 (TBS-T). Transferred membranes were incubated for MMV N, P and GFP accumulation with anti-N (1:5000), anti-P (1:5000), anti-virion (1:2000) and anti-GFP (Invitrogen, MA5-15256-HRP; 1:2,000 dilution) respectively overnight at 4° C. with gentle shaking and then washed 3 times with TBS-T before adding secondary antibody goat anti-rabbit (whole virion and N) and goat anti-goat (P) IgG-conjugated alkaline phosphatase for 2 h at room temperature with gentle shaking, then washed 3 times with TBS-T before addition of SuperSignal™ West Pico PLUS Chemiluminescent Substrate (ThermoFisher). All these procedures were performed according to the manufacturer's instructions. Quantification of band signal intensity was performed with the iBright Imaging system (CL1000, ThermoFisher Scientific). The MMV N and P polyclonal antibodies were prepared from rabbits immunized with synthetic peptides to the N and P proteins (GenScript).

Electron microscopy. Systemically infected leaf tissues were fixed in 2.5% glutaraldehyde and 1% osmium tetroxide (both in 100 mM sodium cacodylate buffer, pH 7.0) as previously described Kong et al., 2014. Following ethanol dehydration, the fixed tissues were then embedded in Spurr's resin (Spurr, 1969) as instructed by the manufacturer (Sigma-Aldrich). Ultrathin sections (90 nm) were cut with a Diatome diamond knife from the embedded tissues using the Leica EM U7 Ultramicrotome (Leica Microsystems, Wetzlar, Germany). The thin sections were then stained with 4% uranyl acetate for 20 minutes followed by lead citrate for 30 seconds. The stained sections were imaged on a ThermoFisher Talos F200X TEM operated at 80 KV to determine virion structure in the infected cells.

Virus transmission. To examine the recombinant MMV-GFP transmission efficiency, two virus acquisition methods were used 1) oral feeding of MMV and MMV-GFP were used to check the transmission efficiency between the recombinant and wild-type MMV. For direct feeding method: fourth stage nymphs were given acquisition on symptomatic MMV and MMV-GFP maize plants for a 2-weeks. The young MMV and MMV-GFP infected newly emerged adults (4 male or female adults/plant) were then transferred to healthy 2-week-old maize plants for 2-weeks. P. maidis fed on healthy maize plants were served as controls. After two weeks of IAP, the insects were removed and moved into insect free cages and the plants were monitored for development of disease symptoms until 28 days post inoculation. Virus infection of the experimental plants were determined by DAS-ELISA using commercial antibodies against MMV (Agdia, Inc. Elkhart, IN, USA) as described previously by Sutula et al., 1986.

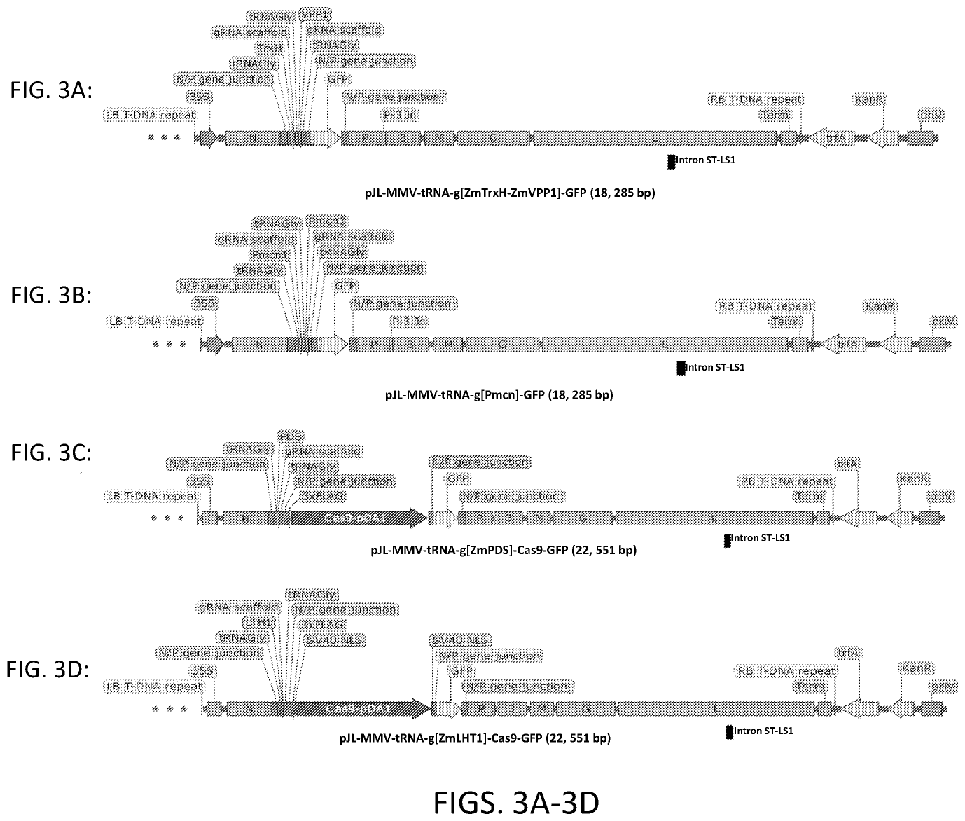

Construction of MMV vector expression of tgtRNA (MMV-gRNA). To alter expression of maize genes, guide RNAs (gRNAs) were designed that contain a 20 bp guide sequence matching a 20 bp region in the Zea mays Thioredoxin H (ZmTrxH), Z. mays vascuolar-type H⁺ pyrophosphatase (ZmVPP1), and Z. mays phytoene desaturase (ZmPDS), Z. mays lysine-histidine transporter (ZmLHT1). To test the genome editing capability in the insect vector, gRNAs were designed to target eye color genes that are easily phenotyped, including P. maidis cinnabar (Pmcn) genes. The MMV-tgtRNA vector for expression of the guide RNA (gRNA) containing ZmTrxH and ZmVPP1 encodes resistance genes to sugarcane mosaic virus (SCMV) and drought tolerance respectively. To generate MMV-tRNA-g[TrXH-VPP1]-tRNA-GFP, a 20 bp region of ZmTrxH and ZmVPP1 containing tRNA-N/P gene sequences was synthesized and cloned into pJL-MMV-GFP between the N and P genes (FIG. 3A). Using this MMV vector expression system, it is possible to multiplex at least two gRNAs within the same vector and obtain CRIPSR-mediated gene activation or suppression studies in the Cas9 transgenic plants. Recently, CRISPR-mediated editing of plant genes was demonstrated using their plant virus based-gRNA vectors in maize (Gentzel et al., 2021) and N. benthamiana (Ma et al., 2020; Mei et al., 2019) plants expressing Cas9 (Ma et al., 2020; Mei et al., 2019). This construct was designed to test enhancement of target gene expression by inoculating gRNA encoding virus (MMV) in transgenic maize expressing dCas9-TV which confers stronger transcriptional activation of a single or multiple target genes (see. e.g., Li et al., Nat Plants. 2017 Dec; 3(12): 930-936; Gentzel et al., Plant Methods vol. 16: 133 (2020)). To test effectiveness of genome editing in planthoppers, the MMV-tgtRNA vector was designed for expression of the guide RNA (gRNA) targeting insect eye-color genes. The P. maidis cinnabar (Pmcn) gene encodes for kynurenine 3′-monooxygenase and gRNAs targeting this gene were inserted between the N and P genes to generate MMV-tRNA-g[Pmcn]-tRNA-GFP (FIG. 3B). The MMV-tgtRNA vector for expression of the guide RNA (gRNA) containing Pmcn in Cas9-transgenic P. maidis will generate red compound eyes. These engineered MMV vectors could deliver multiple gRNAs and may mediate genome editing in Cas9-transgenic maize and P. maidis.

Briefly, partial N-tRNA[TrxH-VPP1]-tRNA-GFP-partial M (4972 bp) containing duplicate N/P gene junctions was synthesised by Genscript. To generate pJL-MMV-tRNA-g[TrXH-VPP1]-tRNA-GFP, plasmids pJL-MMV-GFP and pUC57-tRNA-g[TrXH-VPP1]-RNA-GFP were restricted using Bst1107I and PasI were restricted and 13,386 bp and 4972 bp fragments were ligated using T4 DNA ligase (Thermo Scientific). To generate MMV-tRNA-g[Pmcn]-tRNA-GFP construct, N-tRNA-g[Pmcn]-tRNA-GFP containing two gRNAs of cinnabar and N/P gene junctions were synthesised by Genscript. Plasmids pJL-MMV-GFP, pUC57-N-tRNA-g[Pmcn]-tRNA-GFP plasmid were restricted using Bst1107I and PasI were restricted and 13,386 and 4972 bp fragments ligated using T4 DNA ligase to generate MMV-tRNA-g[Pmcn]-tRNA-GFP construct.

Construction of MMV vector for expression of Cas9. To construct pJET-MMV-N-GFP-M, a PCR fragment (7,575 bp) encompassing partial N gene downstream and partial M gene from pJL-MMV-GFP plasmid using NDraIIIF and MPas1R primers by Q5 high-fidelity DNA polymerase (NEB) and cloned in pJET2.1 blunt vector. To create partial pJET-MMV-N-Cas9-GFP-M construct, PCR fragment 1 containing the entire MMV-N-Cas9-GFP-M (7,575 bp) and fragment 2 containing the entire Cas9 gene amplified from pJET-MMV-N-GFP-M and pDA1-Cas9 plasmids using N-NPF/R and N-pDACas9F/R primers, respectively. Both PCR fragments were assembled using Hifi assembly (NEB). To construct MMV-Cas9 vector, pJET-MMV-N-Cas9-GFP-M and pJL-MMV-GFP plasmids were digested using Bst1170I and PasI restriction enzymes, fragments 8,356 bp and 13,575 bp were ligated using T4 DNA ligase (ThermoScientific). Primers used in this study for construction of this and all subsequent plasmids are listed below in Table 4.

Construction of MMV vector expression of tgtRNA and Cas9 (MMV-tgtRNA-Cas9). The MMV-tgtRNA-Cas9 vector for expression of the gRNA containing Z. mays Lysine/Histidine transporter (ZmLHT1), Z. mays phytoene desaturase (ZmPDS) and Cas9 were generated. Briefly, the partial N-tRNA-LHTI/pds-partial Cas9 and partial N-tRNA-Zmpds-partial Cas9 fragments containing duplicate N/P gene junctions were synthesised in pUC57 separately by Genscript. The pJET-MMV-Cas9 and pUC57-partial N-tRNA-ZmLHT1/ZmPDS-partial Cas9 plasmids were restricted using Bst1170I and Mlu1, two fragments 10,386 bp and 1558 bp were gel eluted and ligated using T4 DNA ligase to generate MMV-tRNA-ZmLHT1-Cas9-GFP (FIG. 3C) and MMV-tRNA-ZmPDS-Cas9-GFP (FIG. 3D) constructs (Thermo Scientific). Using these strategies, MMV-tgtRNA-Cas9) vectors could deliver guide RNA and maize codon optimized Cas9 for maize-targeted gene mutagenesis.

### 4. EXAMPLES

It will be readily apparent to those skilled in the art that other suitable modifications and adaptations of the methods of the present disclosure described herein are readily applicable and appreciable, and may be made using suitable equivalents without departing from the scope of the present disclosure or the aspects and embodiments disclosed herein. Having now described the present disclosure in detail, the same will be more clearly understood by reference to the following examples, which are merely intended only to illustrate some aspects and embodiments of the disclosure, and should not be viewed as limiting to the scope of the disclosure. The disclosures of all journal references, U.S. patents, and publications referred to herein are hereby incorporated by reference in their entireties.

The present disclosure has multiple aspects, illustrated by the following non-limiting examples.

### Example 1

Insertion of plant intron in L gene increased the stability of MMV infectious clone. To generate a wild type MMV cDNA clone, full length cDNA segment was amplified from synthesized pBrick-MMV and cloned into pJL89 between the double cauliflower mosaic virus promoter (2× 35S) and hepatitis delta virus ribozyme sequence (Lindbo, 2007) in vitro by one-step Hifi assembly. During plasmid construction, it was observed that transformed colonies have desired fragment upon colony-PCR with MMV N, P and L gene specific primers, when plasmid was extracted from overnight bacterial culture produced a low quantity of pJL-MMV and pJL-L plasmid and none of the extracted plasmids have generated desired fragments with plasmid PCR with gene specific primers. When three types of E. coli competent cells were used (DH5α (ThermoScientific), Top10 (ThermoScientific) and Stable competent cells (NEB)), and A. tumefaciens GV3101, the unexpected bands were still present. Restriction enzyme digestion showed that plasmids no longer had the desired length of the virus genome. Growing bacterial cultures with plasmids at lower temperatures (25° C. and 30° C.) also did not solve the viral genome stability in E. coli. It was speculated that two possibilities for the instability of full length MMV infectious clone and L gene in E. coli and Agrobacterium included its large size and expression of toxic viral products from MMV genome.

To reduce the plasmid instability and interrupt cytotoxicity caused by the viral sequences, insertion of an intron into the plant virus infectious clones has proven to be an efficient way for viruses belonging to the genera Tobravirus (Ratcliff et al., 2001) and Potyvirus (Johansen, 1996; Lopez-Moya and Garcia, 2000; Sun et al., 2017; Tran et al., 2019). The putative intron splicing sites of wild type L gene sequences was predicted by Alternative Splice Site Predictor (ASSP) (Wang and Marin, 2006). To stabilize the full-length L gene, ST-LSI was inserted between 3281 and 3282 splice site (CTGCGGACAG\hat{ }GTATCGATAT; SEQ ID NO: 12) nucleotides. Then the assembled DNA fragments were transformed into NEB stable competent cells with the same temperature (30° C.) suggested by the manufacturer. When the E. coli transformants were cultured overnight, pJL-L-intron showed a single plasmid with higher yield. Similarly, pJL-MMV-intron showed a single plasmid in overnight liquid culture. Restriction digestion with Xho1 showed the expected bands pattern for both pJL-L-intron and pJL-MMV-intron, showing its stability in prolonged cultures. The pJL-L-intron and pJL-MMV-intron were transformed into A. tumefaciens 3101, which also demonstrated increased stability. E. coli competent cells DH5α Top10 produced single plasmid at 37° C. These results indicate that the presence of plant intron in L genes strongly increased the growth of E. coli and Agrobacterium in liquid cultures at different temperatures.

### Example 2

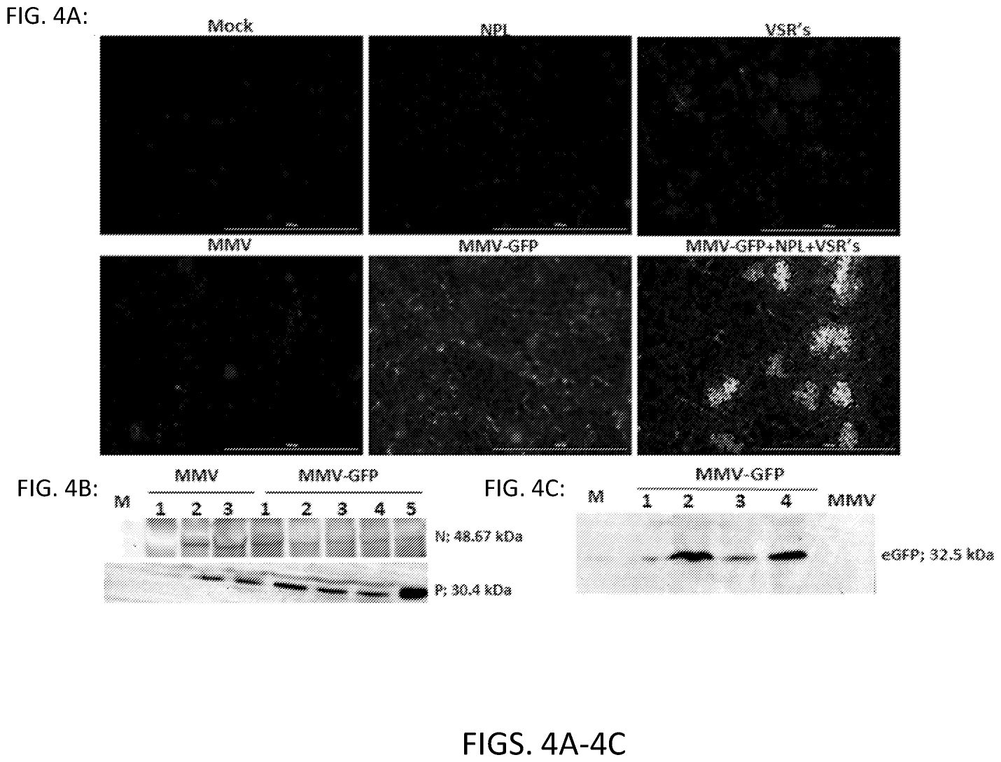

Recovery of infectious MMV from cloned cDNAs in N. benthamiana leaves. To establish a reverse genetic system. NSVs basically require in vivo reconstitution of active replicase complexes including the N, P and L proteins. Thus, a mixture of agrobacterium cultures harbouring the pJL-MMV and the supporting plasmids pTF-N&P and pJL-L-intron along with three viral suppressors genes (VSRs: P19 from tomato bushy stunt virus (TBSV), HcPro from tobacco etch virus (TEV), and γb from barley stripe mosaic virus (BSMV) were co-infiltrated into N. benthamiana leaves. The plants were then monitored for systemic infections. After 12 dpi, RT-PCR using gene specific primers detected MMV P and 3 proteins in the systemic leaves; however no systemic infection was observed in the systemic leaves in replicated experiments. No systemic movement of P and 3 were observed in distal tissues and an absence of virus disease symptoms at 30 dpi. This indicates that wild type MMV failed to move systemically in the infiltrated N. benthamiana leaves. To monitor virus infection in vivo, the MMV vector was engineered with a GFP gene insertion between the N/P gene junction. Plants were agroinfiltrated with pJL-MMV-GFP together with binary expression constructs of N, P, L and 3 VSR gene constructs. At 14 dpi, GFP expression observed in the infiltrated leaves of N. benthamiana plants (FIG. 4A). These results show the evidence of N, P (FIG. 4B) and GFP (FIG. 4C) protein expression in the infiltrated plant leaves and indicates that they functionally interact with the agRNA transcripts to rescue recombinant MMV-GFP in the agroinfiltrated N. benthamiana leaves. No fluorescence was observed in leaves infiltrated with mixtures lacking N, P, and L. These results show that cells containing agRNA, genomic RNA and eGFP mRNA transcripts indicating the occurrence of virus replication and transcription.

Agroinfiltrated N. benthamiana plants inoculated with pJL-MMV or pJL-MMV-GFP +NPL and VSR's did not show any symptom or GFP expression in the systemic leaves; these results show N. benthamiana is not a suitable host for MMV accumulation. In these experiments, when Agrobacterium containing pJL-MMV-GFP, pTF-N&P and pJL-L culture at OD₆₀₀ 0.5 and VSRs at OD₆₀₀ 0.2 were used for agroinfiltration greatly increased the number of cells expressing eGFP in the infiltrated leaves. GFP fluorescence was not observed in the pJL-MMV-GFP infiltrated lacking binary expression constructs of N, P, and L (FIG. 4A). Western blot analyses of infiltrated leaves revealed G, N, P proteins in the infiltrated leaves and MMV-GFP, showed that GFP expression was abundant in MMV-GFP infiltrated leaves. In contrast. GFP was not detected in the leaves agroinfiltrated with MMV-GFP alone and MMV-GFP with VSRs (FIG. 4A).

### Example 3

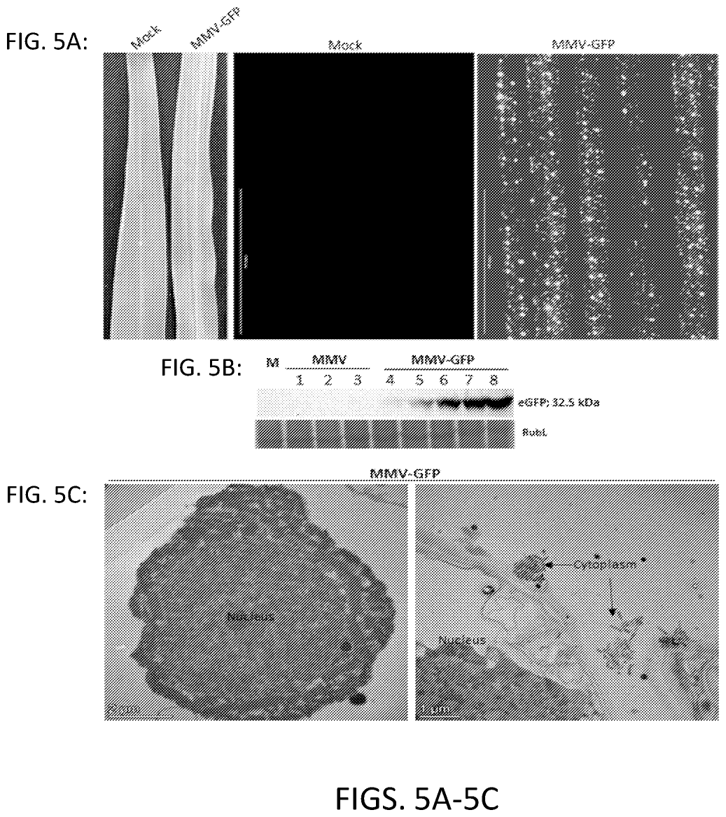

Rescue of wild-type MMV and MMV-GFP in maize and P. maidis. Maize mosaic virus is transmitted in a persistent-propagative manner by P. maidis, and the virus persists in the vector throughout the development (Hogenhout et al., 2008, Barandoc-Alviar et al., 2016). Since N. benthamiana is not an ideal host for P. maidis to acquire the MMV from the infiltrated leaves, 1 g of N. benthamiana leaves agroinfiltrated with pJL-MMV or pJL-MMV-GFP+NPL and VSRs were ground in extraction buffer at 2 weeks dpi and 30 nl of low speed supernatant was injected into thoraxes of healthy P. maidis. After injection, the insects were maintained on healthy maize plants during which 95% of the abdomens from injected adults showed a color change to greenish-brown and eventually died, likely because N. benthamiana is not a host for P. maidis and it is unable to detoxify compounds produced by this plant. Surviving insects and plants were tested for the presence of MMV and MMV-GFP and the virus was unable to be detected. Alternatively, 1 g of N. benthamiana leaves agroinfiltrated with pJL-MMV or pJL-MMV-GFP +NPL and VSRs were ground in a 10 mM phosphate buffer (pH 7) and crude extracts were vascular puncture inoculated to maize kernels. After 14 dpi, plants developed mosaic symptoms on systemic leaves, like those observed in wildtype-MMV infections. In addition, crude extract from N. benthamiana infiltrated with pJL-MMV-GFP+NPL and VSR's was vascular puncture inoculated to maize kernels and resulted in high-intensity GFP fluorescence that was systemically distributed in the young leaves (FIG. 5A). Western blotting verified that abundant GFP accumulated in systemically infected maize leaves (FIG. 5B). Moreover, abundant bacilliform particles were observed in the cytoplasm and nucleus of thin sectioned cells infected with pJL-MMV-GFP. GFP foci were photographed at 24 dpi with a fluorescence microscope (Scale bar: 1000 μm) (FIG. 5C). Three biological replicates results showed the systemic MMV and MMV-GFP infection in vascular puncture inoculated maize kernels. Mock maize kernels vascular puncture inoculated with pJL-MMV or pJL-MMV-GFP alone did not show any symptoms or GFP.

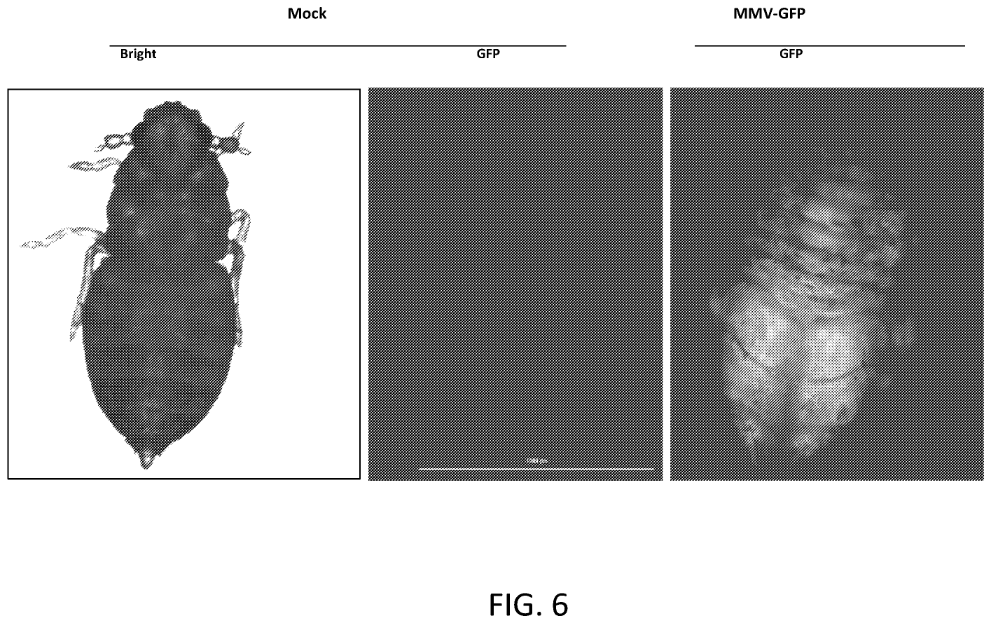

Transmission experiments were performed with P. maidis nymphs injected with MMV-GFP crude extract to determine if the recombinant MMV-GFP is insect transmissible. In the control experiments, P. maidis nymphs injected with maize extracts and released on two-week-old maize plants. In the MMV-GFP crude extract injected nymphs, 0 out of 20 insects tested at 7 dpi and 5 out of 20 (25%) at 14 dpi were positive for L and GFP. After a 28-day post injection incubation period on maize, 9 out of 20 (45%) of surviving adults were tested positive for L and GFP RNA by RT-PCR amplification. The detection of viral RNA after an extended incubation period suggests that the virus is persisting and replicating in the insect. For transmission experiments, after a 28-day incubation period on maize plants, the MMV-GFP planthoppers were transferred to healthy maize plants for a 7-day inoculation access period. After a 21-day post inoculation access period, 12 out of 20 (60%) plants in the first experiment and 13 out of 20 (65%) plants in the second experiment developed mosaic symptoms on systemic leaves. In addition, GFP fluorescence was evident in P. maidis progeny nymphs feeding on MMV-GFP transmitted plants (FIG. 6). Unlike N. benthamiana crude extract, insect vector survival and transmission efficiency were improved by injecting insects with infected maize extracts. These results demonstrate that MMV-GFP rescued in vascular puncture inoculated maize plants could be transmitted to P. maidis by injection of leaf extracts and recombinant MMV-GFP is insect transmissible.

### Example 4

Engineering MMV vector platforms for expressions of foreign proteins in plants and insects. Plant rhabdoviruses are well known for maintaining multiple foreign genes thus enabling stable expression of foreign proteins in plants (Wang et al., 2015; Gao et al., 2019; Ma et al., 2020) and insects (Gao et al., 2019). Here, MMV-based vectors were created for expression of multiple foreign proteins in plants and planthoppers. When healthy maize kernels were VPI inoculated with MMV-GFP showed intense GFP expression in the systemic leaves of maize plants. To further increase simultaneous foreign protein expression, recombinant MMV-GFP vectors were designed to test the efficacy of transgenes for the control of hemipteran insects. Three categories of proteins were tested using MMV vector: 1) B. thuringiensis crystal toxin protein (Cry51Aa2) and 2) spider toxin protein: peptides from H. versuta (Hvt) and 3) lectins form A. cepa agglutinin (ACA). These toxic proteins Cry51Aa2 (Huang et al., 2012) Hvt (Liu et al., 2016) and ACA (Yarasi et al., 2008) are reported with insecticidal activity against sap-feeding hemipteran insects. Genes of interest are inserted between N and P gene junctions to engineer MMV-Cry51Aa2-GFP and MMV-Hvt-ACA-GFP (FIGS. 2A-2B).

### Example 5

Delivery of gRNAs and Cas9 by MMV-based vectors. To investigate the MMV based vectors could deliver the CRISPR-Cas9 nucleases and guide RNAs in maize and insect genome editing, four viral constructs were made: MMV-tRNA-g[TrXH-VPP1]-tRNA-GFP, MMV-tRNA-g[Pmcn]-tRNA-GFP, MMV-tRNA-g[ZmLHT1]-Cas9-GFP and MMV-tRNA-g[ZmPDS]-Cas9-GFP. The MMV-tRNA-g[TrXH-VPP1]-tRNA-GFP construct was designed to test enhancement of target gene expression by inoculating gRNA encoding virus (MMV) in transgenic maize expressing dCas9-TV which confers stronger transcriptional activation of a single or multiple target genes (Li et al., 2017; Gentzel et al., 2020). To test effectiveness of genome editing in planthoppers, the MMV-tRNA-g[Pmcn]-tRNA-GFP vector was designed for expression of the guide RNA (gRNA) targeting insect eye-color genes and it was tested in Cas9-expressing planthoppers. The MMV-tRNA-g[ZmPDS]-Cas9-GFP construct was designed to express a gRNA targeting phytoene desaturase and the Cas nuclease to show genome editing is capable from virally encoded RNA and protein. The MMV-tRNA-g[ZmPDS]-Cas9-GFP construct was designed to express a gRNA targeting phytoene desaturase and the Cas nuclease to show genome editing is capable from virally encoded RNA and protein. The MMV-tRNA-g[ZmLHT1]-Cas9-GFP construct was designed to express a gRNA the to edit the maize LHT1 gene, a recessive resistance gene for southern corn leaf blight, and the Cas nuclease to show genome editing is capable from virally encoded RNA and protein. Constructs were agroinfiltrated into N. benthamiana together with binary expression constructs of N, P, L and 3 VSR gene constructs. N. benthamiana crude extract was vascular puncture inoculated to maize kernels and assayed for systemic high-intensity GFP fluorescence in the agroinfiltrated leaves inoculated individually with MMV-tRNA-g[TrXH-VPP1]-tRNA-GFP, MMV-tRNA-g[Pmcn]-tRNA-GFP, MMV-tRNA-g[ZmLHT1]-Cas9-GFP and MMV-(RNA-g[ZmPDS]-Cas9-GFP constructs (FIG. 3A-3D). Maize genome editing experiments are under progress to demonstrate conclusively that MMV-based vectors could simultaneously deliver gRNA and CRISPR-Cas9 nucleases in maize. P. maidis genome editing experiments are in progress to demonstrate conclusively that MMV-based vectors can deliver gRNA and through interaction with Cas9 edit genomes of recalcitrant insects.

### Example 6

Stable GFP expression by MMV vector in maize and planthoppers following virus passages. Experiments were conducted to analyze foreign gene longevity using RNA extraction and RT-PCR. Leaves of vascular puncture inoculated MMV-infected maize plants and MMV infected maize crude sap injected adult P. maidis were harvested for RNA extraction using the RNeasy Plant Mini kit (Qiagen). After first strand cDNA synthesis, primer pairs MMV N F&R, GFP F&R were used to detect the presence of MMV by RT-PCR. Zea mays actin and P. maidis EF1 genes were used as an internal control with primer pairs Zmactin F&R and PmEF1 F&R respectively. Passages: 1) N. benthamiana to maize, 2) maize to maize, 3) maize to P. maidis and 4) P. maidis to maize. Multiple successive passage experiments were performed. MMV-GFP infected maize plants and planthoppers were collected from the three independent experiments in each passage generation for RNA extraction and RT-PCR analysis.

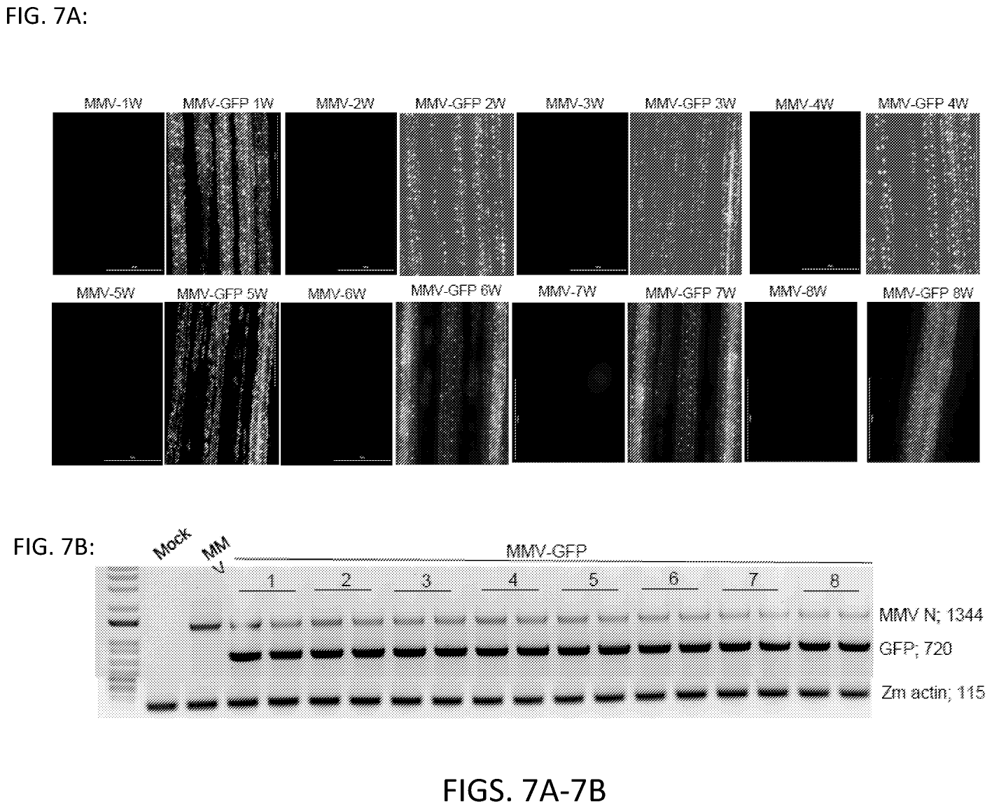

As shown in FIG. 7. results demonstrated that the MMV-GFP vector can be successfully used to express the GFP in the following virus passages: from N. benthamiana to maize, from maize to maize, from maize to P. maidis, and from P. maidis to maize (Table 3). In these experiments, the stability of inserted GFP genes was tested in plants and planthoppers by evaluating the following three successive passages by RT-PCR using MMV N (MMNF: ATGGCAAACATCAACATCC (SEQ ID NO: 35) and MMNR: CTATAAGCCTGATCGTGTCTTC (SEQ ID NO: 36)) and GFP specific (GFPF: ATGGTGAGCAAGGGCGAG (SEQ ID NO: 37) and GFPR: CTTGTACAGCTCGTCC (SEQ ID NO: 38)) primers. During N. benthamiana to maize, and maize to maize passages, infected leaves were collected after 30 dpi and were used as inoculum for RNA extractions and crude sap preparations. Virus passage maize to P. maidis: MMV-GFP infected crude sap was injected to insects and collected after two weeks post release on maize plants were used for RNA extractions. Virus passage P. maidis to maize: next generation P. maidis nymphs and adults feeding on MMV-GFP infected plants were used for RNA extractions. As a reference gene control, maize actin gene and P. maidis elongation factor 1 genes were used. Furthermore, GFP fluorescence in the MMV-infected plants were monitored until the plants died (FIG. 7).

When GFP was evaluated in 10 of the 10 symptomatic plants, all three passages tested positive. The presence of full length MMV GFP bands in all the three passage generations in both plants and planthoppers demonstrated insert stability in the MMV vectors (FIG. 7B). Moreover, frozen MMV-GFP infected tissue crude extracts were also used for injections, which also resulted in successful virus infection in insects and virus transmission.

TABLE 3
Stable GFP expression by MMV vector in N. benthamiana, maize and planthopper following virus passages.

| | N. benthamiana to maize | | | maize to maize | | | maize to P. maidis | | P. maidis to maize | | |
|---|---|---|---|---|---|---|---|---|---|---|---|
| | | symptomatic | | samples | symptomatic | symptomatic | samples | | maize plants | symptomatic | symptomatic |
| | plants | samples | symptomatic | positive | samples | samples | positive | samples | infected via | samples | samples |
| Exp. | infected | positive for | positive for | for | positive for | positive for | for | positive | insect | positive for | positive for |
| No. | via VPI | MMV | GFP | MMV | MMV | GFP | MMV | for GFP | transmission | MMV | GFP |
| 1 | 1/80 | 4/4 | 4/4 | 6/120 | 6/6 | 6/6 | 3/10 | 3/10 | 12/20 | 12/12 | 12/12 |
| | | | | | | | | | (60%) | | |
| 2 | 3/80 | 6/6 | 6/6 | 8/100 | 8/8 | 8/8 | 4/10 | 4/10 | 13/20 | 13/13 | 13/13 |
| | | | | | | | | | (65%) | | |
| 3 | 4/80 | 4/4 | 4/4 | 9/120 | 9/9 | 9/9 | 4/10 | 4/10 | 14/20 | 14/14 | 14/14 |
| | | | | | | | | | (70%) | | |

### Example 7

Target gene gRNA cloning into MMV derived vectors. To alter expression of maize genes, gRNAs were designed that contain a 20 bp guide sequence matching a 20 bp region corresponding to each target: Zea mays Thioredoxin H (ZmTrxH), Z. mays vascuolar-type H⁺ pyrophosphatase (ZmVPP1), Z. mays phytoene desaturase (ZmPDS), and Z. mays lysine-histidine transporter (ZmLHT1) (see FIGS. 3A-3D). To test the genome editing capability in the insect vector, gRNAs were designed to target eye color genes that are easy to phenotype, including P. maidis cinnabar (Pmcn) genes. The MMV-tgtRNA vector for expression of the guide RNA (gRNA) containing ZmTrxH and ZmVPP1 encodes resistance genes to sugarcane mosaic virus (SCMV) and drought tolerance respectively. To generate MMV-tRNA-g[TrXH-VPP1]-tRNA-GFP, a 20 bp region of ZmTrxH and ZmVPP1 containing tRNA-N/P gene sequences was synthesized and cloned into pJL-MMV-GFP between the N and P genes (FIG. 3)

Similarly, a MMV-tgtRNA vector for expression of a gRNA containing eye-color genes, P. maidis cinnabar (Pmcn) encodes for kynurenine 3′-monooxygenase between the N and P genes to generate MMV-tRNA-g[Pmcn]-tRNA-GFP (FIG. 3). The MMV-tgtRNA vector for expression of the gRNA containing Pmcn in Cas9-transgenic P. maidis will generate red compound eyes respectively. These engineered MMV vectors can deliver multiple gRNAs and can mediate genome editing in Cas9-transgenic maize and P. maidis.

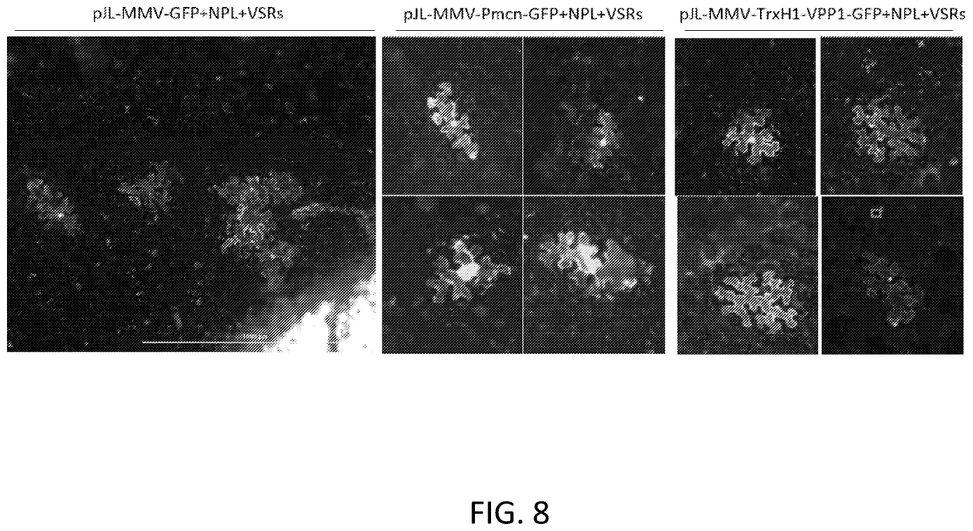

GFP expressing N. benthamiana leaf extracts infiltrated with MMV-GFP/MMV-Pmcn-GFP/MMV-TrxH1-VPP1-GFP plasmids were homogenized in extraction buffer (10 mM Potassium buffer; pH 7) and centrifuged at 12,000 g for 5 min at 4° C. Then 5 ul of crude supernatants were vascular puncture inoculated using a 5 pin-tattoo needle into the overnight soaked maize kernels at 30° C. Then the VPI inoculated seeds were incubated in a humid tray at 30° C. for two days before sowing. These plants were subsequently observed for systemic symptom expression or image analysis. As shown in FIG. 8, GFP expression at 10 dpi was observed in the infiltrated leaves of N. benthamiana plants with MMV-GFP/MMV-Pmcn-GFP/MMV-TrxH1-VPP1-GFP constructs with NPL+VSRs.

These results demonstrate N, P, L, and GFP protein expression in the infiltrated plant leaves and indicates that they functionally interact with the agRNA transcripts to rescue recombinant MMV-GFP/MMV-Pmcn-GFP/MMV-TrxH1-VPP1-GFP in the agroinfiltrated N. benthamiana leaves. GFP expression was observed in the infiltrated leaves of N. benthamiana plants. No fluorescence was observed in leaves infiltrated with mixtures lacking N, P, and L. These results demonstrate that cells containing agRNA, genomic RNA, and eGFP mRNA transcripts indicated the occurrence of virus replication and transcription.

Sequences. The various embodiments of the present disclosure described herein may include one or more of the sequences referenced below, which can be found in the corresponding sequence listing.

TABLE 4
Primers (ssequences in lowercase letters are designed to facilitate Hi-fi
assembly).

| | | | SEQ ID |
|---|---|---|---|
| Primer | Sequence (5′ → 3′) | Application | NO: |
| pJLF | GGGTCGGCATGGCATCTC | Construction of pJL-MMV | 13 |
| pJLR | CCTCTCCAAATGAAATGAACTTC | Construction of pJL-MMV | 14 |
| | C | | |
| pJLMMVF | catttcatttggagaggAGAGACCCATATA | Construction of pJL-MMV | 15 |
| | TTTCATAAA | | |
| pJLMMVR | gagatgccatgccgacccAGAGACCCAGA | Construction of pJL-MMV | 16 |
| | AAAACATGGC | | |
| MMVNF | ATGGCAAACATCAACATCC | Construction of pTF-N&P | 17 |
| MMVNR | CTATAAGCCTGATCGTGTCTTC | Construction of pJL-N&P | 18 |
| MMVPF | ATGAATCGTTACTCCCGTCG | Construction of pTF-N&P | 19 |
| MMVPR | TTAGATCTCAACCCTAGGGC | Construction of pJL-N&P | 20 |
| 35SLF | catttcatttggagaggATGGATCCAGATT | Construction of pJL-L- | 21 |
| | ATCCTGATC | intron | |
| TerLF | AATTCCTAAAACCAAAATCCAGT | Construction of pJL- | 22 |
| | | MMV-intron and pJL-L- | |
| | | intron | |
| TerLR | gagatgccatgccgacccTCACCCGAGCA | Construction of pJL-L- | 23 |
| | GGTCGGACAC | intron | |
| MMVLXSR | GTCGGATCTCATTCAGTCCATGTG | Construction of pJL- | 24 |
| | TGGGGGCGTC | MMV-intron and pJL-L- | |
| | | intron | |
| LXSF | GACGCCCCCACACATGGACTGAA | Construction of pJL- | 25 |
| | TGAGATCCGAC | MMV-intron and pJL-L- | |
| | | intron | |
| MMV1355F | TGTCTGCAGGTGGAGACGAGGCA | Detection of gene inserts | 26 |
| | | between N and P | |
| NDraIIIF | CATTCAGTGCGACGCATCTCAG | Construction of pJET- | 27 |
| | | MMV-N-GFP-M | |
| MpasIF | AGACCGTCATCTTCTCCC | Construction of pJET- | 28 |
| | | MMV-N-GFP-M | |
| N-NPF | ATCTCAGCTCGGTATCAATA | Construction of MMV- | 29 |
| | | Cas9 | |
| N-NPR | CTATAAGCCTGATCGTGTC | Construction of MMV- | 30 |
| | | Cas9 | |
| N-pDACas9F | GACACGATCAGGCTTATAGATGG | Construction of MMV- | 31 |
| | ACTATAAGGATCACGA | Cas9 | |
| N-pDACas9R | TATTGATACCGAGCTGAGATTCA | Construction of MMV- | 32 |
| | CCCGCCAACTTTCCTC | Cas9 | |
| MMV3F | ATGGAGCCCTCTGCAGAGATCCC | RT-PCR detection of | 33 |
| | CC | MMV 3 | |
| MMV3R | TATGGAGGAGGGACCTGTTATGA | RT-PCR detection of | 34 |
| | TGGTG | MMV 3 | |
| MMNF | ATGGCAAACATCAACATCC | RT-PCR detection of | 35 |
| | | MMV-GFP | |
| MMNR | CTATAAGCCTGATCGTGTCTTC | RT-PCR detection of | 36 |
| | | MMV-GFP | |
| GFPF | ATGGTGAGCAAGGGCGAG | RT-PCR detection of | 37 |
| | | MMV-GFP | |
| GFPR | CTTGTACAGCTCGTCC | RT-PCR detection of | 38 |
| | | MMV-GFP | |

What is claimed is:

1. A recombinant viral expression vector comprising:
a negative strand RNA virus (NSV) backbone comprising polynucleotide sequences encoding core proteins; and
at least one expression cassette comprising an exogenous polynucleotide sequence flanked by viral gene junctions from the NSV backbone;
wherein the NSV backbone encodes a virus capable of infecting a target organism and expressing the exogenous polynucleotide sequence.

2. The vector of claim 1, wherein the core proteins comprise a nucleoprotein (N), a phosphoprotein (P), a matrix protein (M), a glycoprotein (G), and a polymerase protein (L).

3. The vector of claim 1 or claim 2, wherein the N protein is upstream of the other core proteins.

4. The vector of any of claims 1 to 3, wherein the L protein is downstream of the other core proteins.

5. The vector of any of claims 1 to 4, wherein the L protein comprises at least one intronic sequence.

6. The vector of claim 5, wherein the at least one intronic sequence enhances stability of the vector.

7. The vector of claim 5, wherein the at least one intronic sequence is inserted into the L protein at a splice site or a predicted splice site.

8. The vector of claim 5, wherein the at least one intronic sequence is heterologous.

9. The vector of any of claims 1 to 8, wherein the at least one expression cassette is positioned between the N protein and the P protein.

10. The vector of any of claims 1 to 9, wherein the viral gene junctions flanking the at least one expression cassette are N/P junctions.

11. The vector of any of claims 1 to 10, wherein the vector comprises at least a second expression cassette comprising a second exogenous polynucleotide sequence.

12. The vector of claim 11, wherein the second expression cassette is flanked by N/P junctions.

13. The vector of any of claims 1 to 12, wherein the first exogenous polynucleotide sequence encodes a first gene-of-interest.

14. The vector of any of claims 1 to 13, wherein the second exogenous polynucleotide sequence encodes a second gene-of-interest.

15. The vector of any of claims 1 to 14, wherein the first or the second exogenous polynucleotide encodes at least one component of a CRISPR-Cas system.

16. The vector of claim 15, wherein the CRISPR-Cas system is a genome engineering system, a CRISPRa system, a CRISPRi system, a base editing system, a prime editing system, or a gap editing system.

17. The vector of claim 15 or claim 16, wherein the CRISPR-Cas system is selected from the group consisting of a Type I CRISPR-Cas system, a Type II CRISPR-Cas system, a Type III CRISPR-Cas system, and a Type V CRISPR-Cas system.

18. The vector of any of claims 15 to 17, wherein:
(i) the system is a Type I CRISPR-Cas system, and the at least one component is Cas3;
(ii) the system is a Type II CRISPR-Cas system, and the at least one component is Cas9;
(iii) the system is a Type III CRISPR-Cas system, and the at least one component is Csm (III-A) or Cmr (III-B); or
(iv) the system is a Type V CRISPR-Cas system, and the at least one component is Cas12a.

19. The vector of any of claims 1 to 18, wherein the first or the second exogenous polynucleotide encodes at least one guide RNA (gRNA).

20. The vector of any of claims 1 to 18, wherein the first or the second exogenous polynucleotide encodes a fluorescent protein.

21. The vector of any of claims 1 to 20, wherein the vector further comprises a promoter and/or a terminator.

22. The vector of any of claims 1 to 21, wherein the NSV backbone is derived from a virus from the genus Alphanucleorhabdovirus.

23. The vector of any of claims 1 to 22, wherein target organism is a plant.

24. The vector of any of claims 1 to 22, wherein target organism is an insect.

25. The vector of claim 24, wherein the plant is selected from the group consisting of maize, wheat, potato, caneberry, eggplant, pepper, tomato, potato, sorghum, rice, taro, alfalfa, peanut, black currant, barley, oat, broccoli, cabbage, kale, lettuce, papaya, strawberry, coffee, citrus, orchid, and pulses (beans and lentils), and variants thereof.

26. The vector of claim 24 or claim 25, wherein the vector is transmissible to insects.

27. A cell comprising any of the vectors of claims 1 to 26, wherein the cell is selected from the group consisting of a plant cell, an insect cell, a yeast cell, and a bacterial cell.
